(19) United States
(12) Patent Application Publication
(10) Pub. No.: US 2010/0041760 A1
(43) Pub. Date: Feb. 18, 2010

(54) MONOARYL AMINOTETRALINES

(76) Inventors: Jean-Baptiste Blanc, Westfield, NJ (US); Li Chen, Shanghai (CN); Fariborz Firooznia, Florham Park, NJ (US); Paul Gillespie, Westfield, NJ (US); Robert Alan Goodnow, JR., Gillette, NJ (US); Tai-An Lin, Pequannock, NJ (US); Song Pan, Shanghai (CN); Sung-Sau So, Verona, NJ (US); HongYing Yun, Shanghai (CN)

Correspondence Address: HOFFMANN-LA ROCHE INC.;PATENT LAW DEPARTMENT, 340 KINGSLAND STREET, NUTLEY, NJ, 07110, US

(21) Appl. No.: 12/540,780

(22) Filed: Aug. 13, 2009

Related U.S. Application Data
(60) Provisional application No. 61/089,116, filed on Aug. 15, 2008.

(51) Int. Cl. A61K 31/18; C07C 311/00

(52) U.S. Cl. 514/604; 564/92

(57) ABSTRACT

The invention is concerned with the compounds of formula I:

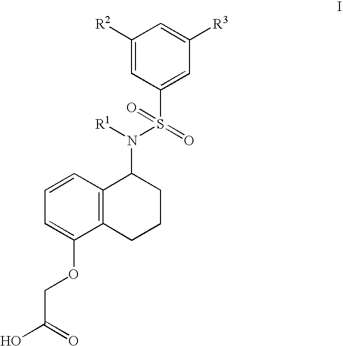

and pharmaceutically acceptable salts and esters thereof, wherein R¹, R² and R³ are defined in the detailed description and claims. In addition, the present invention relates to methods of manufacturing and using the compounds of formula I as well as pharmaceutical compositions containing such compounds. The compounds of formula I are antagonists at the CRTH2 receptor and may be useful in treating diseases and disorders associated with that receptor such as asthma.

### PRIORITY TO RELATED APPLICATIONS

This application claims the benefit of U.S. Provisional Application No. 61/089,116, filed Aug. 15, 2008, which is hereby incorporated by reference in its entirety.

### BACKGROUND OF THE INVENTION

The present invention relates to novel (5-amino-5,6,7,8-tetrahydro-naphthalene-1-yloxy)-acetic acids, their manufacture, pharmaceutical compositions containing them and their use as CRTH2 antagonists.

Prostaglandin D₂ (PGD2) is the major prostanoid produced by activated mast cells and has been implicated in the pathogenesis of allergic diseases such as allergic asthma and atopic dermatitis. Chemoattractant Receptor-homologous molecule expressed on T-helper type cells (CRTH2) is one of the prostaglandin D₂ receptors and is expressed on the effector cells involved in allergic inflammation such as T helper type 2 (Th2) cells, eosinophils, and basophils (Nagata et al., FEBS Lett 459: 195-199, 1999). It has been shown to mediate PGD2-stimulated chemotaxis of Th2 cells, eosinophils, and basophils (Hirai et al., J Exp Med 193: 255-261, 2001). Moreover, CRTH2 mediates the respiratory burst and degranulation of eosinophils (Gervais et al., J Allergy Clin Immunol 108: 982-988, 2001), induces the production of proinflammatory cytokines in Th2 cells (Xue et al., J Immunol 175: 6531-6536), and enhances the release of histamine from basophils (Yoshimura-Uchiyama et al., Clin Exp Allergy 34:1283-1290). Sequence variants of the gene encoding CRTH2, which differentially influence its mRNA stability, are shown to be associated with asthma (Huang et al., Hum Mol Genet 13, 2691-2697, 2004). Increased numbers of circulating T cells expressing CRTH2 have also been correlated with severity of atopic dermatitis (Cosmi et al., Eur J Immunol 30, 2972-2979, 2000). These findings suggest that CRTH2 plays a proinflammatory role in allergic diseases. Therefore, antagonists of CRTH2 are believed to be useful for treating disorders such as asthma, allergic inflammation, chronic obstructive pulmonary disease (COPD), allergic rhinitis, and atopic dermatitis.

### SUMMARY OF THE INVENTION

The invention is concerned with the compounds of formula I:

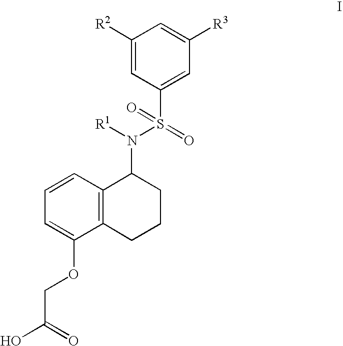

and pharmaceutically acceptable salts and esters thereof, wherein R¹, R² and R³ are defined in the detailed description and claims. In addition, the present invention relates to methods of manufacturing and using the compounds of formula I as well as pharmaceutical compositions containing such compounds. The compounds of formula I are antagonists at the CRTH2 receptor and may be useful in treating diseases and disorders associated with that receptor such as asthma.

### DETAILED DESCRIPTION OF THE INVENTION

Unless otherwise indicated, the following specific terms and phrases used in the description and claims are defined as follows:

The term “moiety” refers to an atom or group of chemically bonded atoms that is attached to another atom or molecule by one or more chemical bonds thereby forming part of a molecule. For example, the variables R¹, R² and R³ of formula I refer to moieties that are attached to the core structure of formula I by a covalent bond.

In reference to a particular moiety with one or more hydrogen atoms, the term “substituted” refers to the fact that at least one of the hydrogen atoms of that moiety is replaced by another substituent or moiety. For example, the term “lower alkyl substituted by halogen” refers to the fact that one or more hydrogen atoms of a lower alkyl (as defined below) is replaced by one or more halogen atoms (i.e, trifluoromethyl, difluoromethyl, fluoromethyl, chloromethyl, etc.). Similarly, the term “lower cycloalkyl substituted by lower alkyl” refers to the fact that one or more hydrogen atoms of a lower cycloalkyl (as defined below) is replaced by one or more lower alkyls (i.e, 1-methyl-cyclopropyl, 1-ethyl-cyclopropyl, etc.).

The term “optionally substituted” refers to the fact that one or more hydrogen atoms of a moiety (with one or more hydrogen atoms) can be, but does not necessarily have to be, substituted with another substituent.

The term “alkyl” refers to an aliphatic straight-chain or branched-chain saturated hydrocarbon moiety having 1 to 20 carbon atoms. In particular embodiments the alkyl has 1 to 10 carbon atoms.

The term “lower alkyl” refers to an alkyl moiety having 1 to 7 carbon atoms. In particular embodiments the lower alkyl has 1 to 4 carbon atoms and in other particular embodiments the lower alkyl has 1 to 3 carbon atoms. Examples of lower alkyls include methyl, ethyl, propyl, isopropyl, butyl, isobutyl, sec-butyl and tert-butyl.

The term “lower cycloalkyl” refers to a saturated or partly unsaturated non-aromatic hydrocarbon ring moiety having 3 to 7 carbon atoms bonded together to form a ring structure. Examples of cycloalkyls include cyclopropyl, cyclobutyl, cyclopentyl, cyclohexyl and cycloheptyl.

The term “lower alkenyl” refers to an aliphatic straight-chain or branched-chain hydrocarbon moiety having 2 to 7 carbon atoms and having at least one carbon-to-carbon double bond. In particular embodiments the lower alkenyl has 2 to 4 carbon atoms, and in other embodiments, 2 to 3 carbon atoms. Examples of lower alkenyls include ethenyl, 1-propenyl, 2-propenyl, 1-butenyl, 2-butenyl, 3-butenyl and isobutenyl.

The term “lower alkoxy” refers to the moiety —O—R, wherein R is lower alkyl as defined previously. Examples of lower alkoxy moieties include methoxy, ethoxy, n-propoxy, isopropoxy, n-butoxy, isobutoxy, sec-butoxy and tert-butoxy.

The term “lower cycloalkoxy” refers to the moiety —O—R, wherein R is lower cycloalkyl as defined previously. Examples of lower cycloalkoxy moieties include cyclobutoxy and cyclopentoxy.

The term “lower alkanoyl” refers to the moiety —C(O)—R, wherein R is lower alkyl as defined previously. An example of a lower alkanoyl is acetyl.

The term “heteroatom” refers to nitrogen, oxygen, or sulfur.

The term “lower heterocycloalkyl” refers to a saturated or partly unsaturated non-aromatic ring moiety having 3 to 7 ring atoms bonded together to form a ring structure wherein one, two or three of the ring atoms is a heteroatom while the remaining ring atoms are carbon atoms. Examples of lower heterocycloalkyls include piperidinyl, piperazinyl, pyrrolidinyl and tetrahydropyran-4-yl.

The term “lower heterocycloalkyloxy” refers to the moiety R′—O—, wherein R′ is a lower heterocycloalkyl as defined above. An example of a lower heterocycloalkyloxy is tetrahydropyran-4-yloxy.

The term “lower alkylsulfanyl” refers to the moiety —S—R, wherein R is lower alkyl as defined previously. Examples of lower alkylsulfanyls include methylsulfanyl and ethylsulfanyl.

The term “lower cycloalkylsulfanyl” refers to the moiety —S—R, wherein R is lower cycloalkyl as defined previously. Examples of lower cycloalkylsulfanyls include cyclopropylsulfanyl, cyclobutylsulfanyl and cyclopentylsulfanyl.

The term “lower heterocycloalkylsulfanyl” refers to the moiety —S—R, wherein R is lower heterocycloalkyl as defined previously. An example of a lower heterocycloalkylsulfanyl is pyrrolidin-1-ylsulfanyl.

The term “lower alkylsulfinyl” refers to the moiety —S(O)—R, wherein R is lower alkyl as defined previously. Examples of lower alkylsulfinyls include methylsulfinyl and ethylsulfinyl.

The term “lower cycloalkylsulfinyl” refers to the moiety —S(O)—R, wherein R is lower cycloalkyl as defined previously. Examples of lower cycloalkylsulfinyls include cyclopropylsulfinyl, cyclobutylsulfinyl and cyclopentylsulfinyl.

The term “lower heterocycloalkylsulfinyl” refers to the moiety —S(O)—R, wherein R is lower heterocycloalkyl as defined previously. An example of a lower heterocycloalkylsulfinyl is pyrrolidin-1-ylsulfinyl.

The term “lower alkylsulfonyl” refers to the moiety —S(O)₂—R, wherein R is lower alkyl as defined previously. Examples of lower alkylsulfonyls include methylsulfonyl and ethylsulfonyl.

The term “lower cycloalkylsulfonyl” refers to the moiety —S(O)₂—R, wherein R is lower cycloalkyl as defined previously. Examples of lower cycloalkylsulfonyls include cyclopropylsulfonyl, cyclobutylsulfonyl and cyclopentylsulfonyl.

The term “lower heterocycloalkylsulfonyl” refers to the moiety —S(O)₂—R, wherein R is lower heterocycloalkyl as defined previously. An example of a lower heterocycloalkylsulfonyl is pyrrolidin-1-ylsulfonyl.

The term “lower alkylsulfonylamino” refers to the moiety —N(H)S(O)₂R, wherein R is lower alkyl as defined previously. Examples of lower alkylsulfonylaminos include methylsulfonylamino and ethylsulfonylamino.

The term “lower alkylamino” refers to the moiety —N(R), wherein R is lower alkyl as defined previously. An example of a lower alkylamino is methylamino.

The term “lower dialkylamino” refers to the moiety —N(R)(R′), wherein R and R′ are lower alkyl as defined previously. An example of a lower dialkylamino is dimethylamino.

The term “lower trialkylsilyl” refers to the moiety —Si(R)(R′)(R″) wherein R, R′ and R″ are lower alkyl as defined previously. An example of a lower trialkylsilyl is trimethylsilyl.

The term “halogen” refers to a moiety of fluoro, chloro, bromo or iodo.

Unless otherwise indicated, the term “hydrogen” or “hydro” refers to the moiety of a hydrogen atom (—H) and not H₂.

Unless otherwise indicated, the term “a compound of the formula” or “a compound of formula” or “compounds of the formula” or “compounds of formula” refers to any compound selected from the genus of compounds as defined by the formula (including any pharmaceutically acceptable salt or ester of any such compound).

The term “pharmaceutically acceptable salts” refers to those salts which retain the biological effectiveness and properties of the free bases or free acids, which are not biologically or otherwise undesirable. Salts may be formed with inorganic acids such as hydrochloric acid, hydrobromic acid, sulfuric acid, nitric acid, phosphoric acid and the like, preferably hydrochloric acid, and organic acids such as acetic acid, propionic acid, glycolic acid, pyruvic acid, oxalic acid, maleic acid, malonic acid, salicylic acid, succinic acid, fumaric acid, tartaric acid, citric acid, benzoic acid, cinnamic acid, mandelic acid, methanesulfonic acid, ethanesulfonic acid, p-toluenesulfonic acid, N-acetylcystein and the like. In addition, salts may be prepared by the addition of an inorganic base or an organic base to the free acid. Salts derived from an inorganic base include, but are not limited to, the sodium, potassium, lithium, ammonium, calcium, and magnesium salts and the like. Salts derived from organic bases include, but are not limited to salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, lysine, arginine, N-ethylpiperidine, piperidine, polyamine resins and the like.

The compounds of the present invention can be present in the form of pharmaceutically acceptable salts. The compounds of the present invention can also be present in the form of pharmaceutically acceptable esters (i.e., the methyl and ethyl esters of the acids of formula I to be used as prodrugs). The compounds of the present invention can also be solvated, i.e. hydrated. The solvation can be effected in the course of the manufacturing process or can take place i.e. as a consequence of hygroscopic properties of an initially anhydrous compound of formula I (hydration).

Compounds that have the same molecular formula but differ in the nature or sequence of bonding of their atoms or the arrangement of their atoms in space are termed “isomers.” Isomers that differ in the arrangement of their atoms in space are termed “stereoisomers.” Diastereomers are stereoisomers with opposite configuration at one or more chiral centers which are not enantiomers. Stereoisomers bearing one or more asymmetric centers that are non-superimposable mirror images of each other are termed “enantiomers.” When a compound has an asymmetric center, for example, if a carbon atom is bonded to four different groups, a pair of enantiomers is possible. An enantiomer can be characterized by the absolute configuration of its asymmetric center or centers and is described by the R- and S-sequencing rules of Cahn, Ingold and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The term “a therapeutically effective amount” of a compound means an amount of compound that is effective to prevent, alleviate or ameliorate symptoms of disease or prolong the survival of the subject being treated. Determination of a therapeutically effective amount is within the skill in the art. The therapeutically effective amount or dosage of a compound according to this invention can vary within wide limits and may be determined in a manner known in the art. Such dosage will be adjusted to the individual requirements in each particular case including the specific compound(s) being administered, the route of administration, the condition being treated, as well as the patient being treated. In general, in the case of oral or parenteral administration to adult humans weighing approximately 70 Kg, a daily dosage of about 0.1 mg to about 5,000 mg, 1 mg to about 1,000 mg, or 1 mg to 100 mg may be appropriate, although the lower and upper limits may be exceeded when indicated. The daily dosage can be administered as a single dose or in divided doses, or for parenteral administration, it may be given as continuous infusion.

The term “pharmaceutically acceptable carrier” is intended to include any and all material compatible with pharmaceutical administration including solvents, dispersion media, coatings, antibacterial and antifungal agents, isotonic and absorption delaying agents, and other materials and compounds compatible with pharmaceutical administration. Except insofar as any conventional media or agent is incompatible with the active compound, use thereof in the compositions of the invention is contemplated. Supplementary active compounds can also be incorporated into the compositions.

Useful pharmaceutical carriers for the preparation of the compositions hereof, can be solids, liquids or gases; thus, the compositions can take the form of tablets, pills, capsules, suppositories, powders, enterically coated or other protected formulations (e.g. binding on ion-exchange resins or packaging in lipid-protein vesicles), sustained release formulations, solutions, suspensions, elixirs, aerosols, and the like. The carrier can be selected from the various oils including those of petroleum, animal, vegetable or synthetic origin, e.g., peanut oil, soybean oil, mineral oil, sesame oil, and the like. Water, saline, aqueous dextrose, and glycols are preferred liquid carriers, particularly (when isotonic with the blood) for injectable solutions. For example, formulations for intravenous administration comprise sterile aqueous solutions of the active ingredient(s) which are prepared by dissolving solid active ingredient(s) in water to produce an aqueous solution, and rendering the solution sterile. Suitable pharmaceutical excipients include starch, cellulose, talc, glucose, lactose, talc, gelatin, malt, rice, flour, chalk, silica, magnesium stearate, sodium stearate, glycerol monostearate, sodium chloride, dried skim milk, glycerol, propylene glycol, water, ethanol, and the like. The compositions may be subjected to conventional pharmaceutical additives such as preservatives, stabilizing agents, wetting or emulsifying agents, salts for adjusting osmotic pressure, buffers and the like. Suitable pharmaceutical carriers and their formulation are described in Remington's Pharmaceutical Sciences by E. W. Martin. Such compositions will, in any event, contain an effective amount of the active compound together with a suitable carrier so as to prepare the proper dosage form for proper administration to the recipient.

In the practice of the method of the present invention, an effective amount of any one of the compounds of this invention or a combination of any of the compounds of this invention or a pharmaceutically acceptable salt or ester thereof, is administered via any of the usual and acceptable methods known in the art, either singly or in combination. The compounds or compositions can thus be administered orally (e.g., buccal cavity), sublingually, parenterally (e.g., intramuscularly, intravenously, or subcutaneously), rectally (e.g., by suppositories or washings), transdermally (e.g., skin electroporation) or by inhalation (e.g., by aerosol), and in the form of solid, liquid or gaseous dosages, including tablets and suspensions. The administration can be conducted in a single unit dosage form with continuous therapy or in a single dose therapy ad libitum. The therapeutic composition can also be in the form of an oil emulsion or dispersion in conjunction with a lipophilic salt such as pamoic acid, or in the form of a biodegradable sustained-release composition for subcutaneous or intramuscular administration.

In detail, the present invention relates to the compounds of formula I:

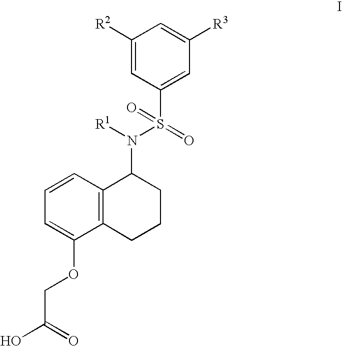

and pharmaceutically acceptable salts and esters thereof, wherein R¹ is (1) hydrogen or (2) methyl optionally substituted by fluoro; and R² and R³ are independently selected from the group consisting of:
(1) halogen;
(2) —NH₂;
(3) —NO₂;
(4) lower alkyl optionally substituted by halogen,
(5) lower cycloalkyl optionally substituted by lower alkyl;
(6) lower alkenyl;
(7) lower alkanoyl;
(8) lower alkoxy;
(9) lower cycloalkoxy;
(10) lower heterocycloalkyl;
(11) lower heterocycloalkyloxy;
(12) lower alkylsulfanyl, lower cycloalkylsulfanyl, or lower heterocycloalkylsulfanyl;
(13) lower alkylsulfinyl, lower cycloalkylsulfinyl, or lower heterocycloalkylsulfinyl;
(14) lower alkylsulfonyl, lower cycloalkylsulfonyl, or lower heterocycloalkylsulfonyl;
(15) lower alkylsulfonylamino;
(16) lower alkylamino;
(17) lower dialkylamino; and
(18) lower trialkylsilyl.

Unless indicated otherwise, the genus of formula I and any subgenera thereof encompass all possible stereoisomers (i.e., (R)-enantiomers and (S)-enantiomers) as well as racemic and scalemic mixtures thereof. In one embodiment of the invention, the compounds of formula I are (R)-enantiomers or pharmaceutically acceptable salts or esters thereof as depicted in the following subgeneric structural formula IA for the (R)-enantiomers of formula I:

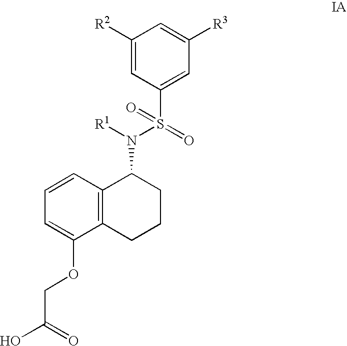

wherein R¹, R² and R³ are as defined previously.

In another embodiment, the compounds of formula I are (S)-enantiomers or pharmaceutically acceptable salts or esters thereof as depicted in the following subgeneric structural formula IB for the (S)-enantiomers of formula I:

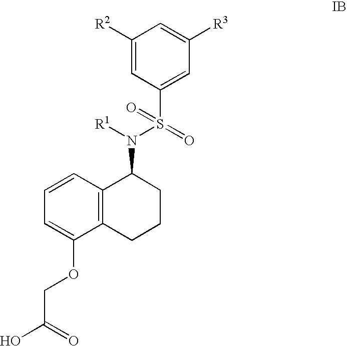

wherein R¹, R² and R³ are as defined previously.

In another embodiment the present invention is directed to a composition comprising a mixture (racemic or otherwise) of the (R)-enantiomers and (S)-enantiomers of a compound of formula I.

In one embodiment the present invention is directed to the compounds of formula I or pharmaceutically acceptable salts or esters thereof, wherein R¹ is hydrogen.

In a more particular embodiment the present invention is directed to the compounds of formula IA or pharmaceutically acceptable salts or esters thereof, wherein R¹ is hydrogen.

In another embodiment the present invention is directed to the compounds of formula I or pharmaceutically acceptable salts or esters thereof, wherein R¹ is methyl.

In a more particular embodiment the present invention is directed to the compounds of formula IA or pharmaceutically acceptable salts or esters thereof, wherein R¹ is methyl.

In another embodiment the present invention is directed to the compounds of formula I or pharmaceutically acceptable salts or esters thereof, wherein R¹ is fluoromethyl.

In a more particular embodiment the present invention is directed to the compounds of formula IA or pharmaceutically acceptable salts or esters thereof, wherein R¹ is fluoromethyl.

In another embodiment the present invention is directed to the compounds of formula I or pharmaceutically acceptable salts or esters thereof, wherein R¹ is difluoromethyl.

In a more particular embodiment the present invention is directed to the compounds of formula IA or pharmaceutically acceptable salts or esters thereof, wherein R¹ is difluoromethyl.

In another embodiment the present invention is directed to the compounds of formula I or pharmaceutically acceptable salts or esters thereof, wherein R¹ is trifluoromethyl.

In a more particular embodiment the present invention is directed to the compounds of formula IA or pharmaceutically acceptable salts or esters thereof, wherein R¹ is trifluoromethyl.

In one embodiment the present invention is directed to the compounds of formula I or pharmaceutically acceptable salts or esters thereof, wherein R² and R³ are independently selected from the group consisting of:

(1) halogen;

(2) lower alkyl;

(3) lower alkyl substituted by halogen;

(4) cycloalkyl;

(5) lower cycloalkyl substituted by lower alkyl;

(6) lower heterocycloalkyl;

(7) lower alkanoyl;

(8) lower alkoxy;

(9) lower cycloalkoxy;

(10) lower alkylsulfinyl;

(11) lower alkylsulfonyl;

(12) lower cycloalkylsulfonyl;

(13) lower alkylamino; and

(14) lower dialkylamino.

In another embodiment, the present invention is directed to the compounds of formula I or pharmaceutically acceptable salts or esters thereof wherein R² or R³ is halogen such as fluoro, chloro, bromo, or iodo. In some specific embodiments R² or R³ is fluoro, chloro, or bromo.

In another embodiment, the present invention is directed to the compounds of formula I or pharmaceutically acceptable salts or esters thereof wherein R² or R³ is lower alkyl such as methyl, ethyl, propyl, isopropyl, butyl, isobutyl, sec-butyl or tert-butyl. In some specific embodiments, R² or R³ is methyl, isopropyl or tert-butyl.

In another embodiment, the present invention is directed to the compounds of formula I or pharmaceutically acceptable salts or esters thereof wherein R² or R³ is lower alkyl substituted by halogen such as trifluoromethyl, difluoromethyl, 1,1-difluoroethyl, or fluoromethyl. In some specific embodiments R² or R³ is 1,1-difluoroethyl or trifluoromethyl. In some more specific embodiments, R² or R³ is trifluoromethyl.

In another embodiment, the present invention is directed to the compounds of formula I or pharmaceutically acceptable salts or esters thereof wherein R² or R³ is lower cycloalkyl such as cyclopropyl, cyclobutyl, cyclopentyl, cyclohexyl or cycloheptyl. In some specific embodiments R² or R³ is cyclopropyl or cyclopentyl.

In another embodiment, the present invention is directed to the compounds of formula I or pharmaceutically acceptable salts or esters thereof wherein R² or R³ is lower cycloalkyl substituted by lower alkyl such as 1-methyl-cyclopropyl or 1-ethyl-cyclopropyl. In some specific embodiments R² or R³ is 1-methyl-cyclopropyl.

In another embodiment, the present invention is directed to the compounds of formula I or pharmaceutically acceptable salts or esters thereof wherein R² or R³ is lower heterocycloalkyl such as piperidinyl, piperazinyl, or pyrrolidinyl. In some specific embodiments R² or R³ is pyrrolidinyl.

In another embodiment, the present invention is directed to the compounds of formula I or pharmaceutically acceptable salts or esters thereof wherein R² or R³ is lower alkanoyl such as propanoyl or acetyl. In some specific embodiments, R² or R³ is acetyl.

In another embodiment, the present invention is directed to the compounds of formula I or pharmaceutically acceptable salts or esters thereof wherein R² or R³ is lower alkoxy such as methoxy, ethoxy, n-propoxy, isopropoxy, n-butoxy, isobutoxy, sec-butoxy or tert-butoxy. In some specific embodiments, R² or R³ is methoxy, ethoxy, or isopropoxy.

In another embodiment, the present invention is directed to the compounds of formula I or pharmaceutically acceptable salts or esters thereof wherein R² or R³ is lower cycloalkoxy such as cyclobutoxy or cyclopentoxy. In some specific embodiments, R² or R³ is cyclopentoxy.

In another embodiment, the present invention is directed to the compounds of formula I or pharmaceutically acceptable salts or esters thereof wherein R² or R³ is lower alkylsulfinyl such as methylsulfinyl, ethylsulfinyl, or isopropylsulfinyl. In some specific embodiments, R² or R³ is isopropylsulfinyl.

In another embodiment, the present invention is directed to the compounds of formula I or pharmaceutically acceptable salts or esters thereof wherein R² or R³ is lower alkylsulfonyl such as methylsulfonyl, ethylsulfonyl, isopropylsulfonyl, or tert-butylsulfonyl. In some specific embodiments, R² or R³ is methylsulfonyl, isopropylsulfonyl or tert-butylsulfonyl.

In another embodiment, the present invention is directed to the compounds of formula I or pharmaceutically acceptable salts or esters thereof wherein R² or R³ is lower cycloalkylsulfonyl such as cyclopropylsulfonyl, cyclobutylsulfonyl or cyclopentylsulfonyl. In some specific embodiments, R² or R³ is cyclopentylsulfonyl.

In another embodiment, the present invention is directed to the compounds of formula I or pharmaceutically acceptable salts or esters thereof wherein R² or R³ is lower alkylsulfonylamino such as methylsulfonylamino or ethylsulfonylamino. In some specific embodiments, R² or R³ is methylsulfonylamino.

In another embodiment, the present invention is directed to the compounds of formula I or pharmaceutically acceptable salts or esters thereof wherein R²or R³ is alkylamino such as methylamino, ethylamino, or isopropylamino. In some specific embodiments, R² or R³ is methylamino.

In another embodiment, the present invention is directed to the compounds of formula I or pharmaceutically acceptable salts or esters thereof wherein R² or R³ is dialkylamino such as dimethylamino, diethylamino, methylethylamino, or methylisopropylamino. In some specific embodiments, R² or R³ is diethylamino or methylisopropylamino.

In one particular embodiment, the present invention is directed to the compounds of formula I or pharmaceutically acceptable salts or esters thereof wherein R¹ is hydrogen and at least one of R² or R³ is trifluoromethyl.

In another particular embodiment, the present invention is directed to the compounds of formula I or pharmaceutically acceptable salts or esters thereof wherein R¹ is methyl and at least one of R² or R³ is trifluoromethyl.

In one particular embodiment, the present invention is directed to the compounds of formula I or pharmaceutically acceptable salts or esters thereof wherein R² and R³ are as defined previously for formula I except that R² and R³ are not both fluoro.

In another particular embodiment, the present invention is directed to the compounds of formula I or pharmaceutically acceptable salts or esters thereof wherein R² and R³ are as defined previously for formula I except that R² and R³ are not both halogen.

In another particular embodiment, the present invention is directed to the compounds of formula I or pharmaceutically acceptable salts or esters thereof wherein R² and R³ are as defined previously for formula I except R² and R³ are not both methyl.

In another particular embodiment, the present invention is directed to the compounds of formula I or pharmaceutically acceptable salts or esters thereof wherein R² and R³ are as defined previously for formula I except that at least one of R² or R³ is neither halogen nor methyl.

In a more specific embodiment, the present invention is directed to a compound of formula I selected from the group consisting of:
[(R)-5-(3,5-dichloro-benzenesulfonylamino)-5,6,7,8-tetrahydro-naphthalen-1-yloxy]-acetic acid;
[(R)-5-(3,5-bis-trifluoromethyl-benzenesulfonylamino)-5,6,7,8-tetrahydro-naphthalen-1-yloxy]-acetic acid;
[(R)-5-(3,5-dimethyl-benzenesulfonylamino)-5,6,7,8-tetrahydro-naphthalen-1-yloxy]-acetic acid;
[(R)-5-(3,5-difluoro-benzenesulfonylamino)-5,6,7,8-tetrahydro-naphthalen-1-yloxy]-acetic acid;
[(R)-5-(3-isopropyl-5-trifluoromethyl-benzenesulfonylamino)-5,6,7,8-tetrahydro-naphthalen-1-yloxy]-acetic acid;
{(R)-5-[3-(1,1-difluoro-ethyl)-5-trifluoromethyl-benzenesulfonylamino]-5,6,7,8-tetrahydro-naphthalen-1-yloxy}-acetic acid;
{(R)-5-[3-(1-methyl-cyclopropyl)-5-trifluoromethyl-benzenesulfonylamino]-5,6,7,8-tetrahydro-naphthalen-1-yloxy}-acetic acid;
[(R)-5-(3,5-di-tert-butyl-benzenesulfonylamino)-5,6,7,8-tetrahydro-naphthalen-1-yloxy]-acetic acid;
[(R)-5-(3,5-bis-methanesulfonyl-benzenesulfonylamino)-5,6,7,8-tetrahydro-naphthalen-1-yloxy]-acetic acid;
[(R)-5-(3-methoxy-5-trifluoromethyl-benzenesulfonylamino)-5,6,7,8-tetrahydro-naphthalen-1-yloxy]-acetic acid;
[(R)-5-(3-bromo-5-trifluoromethyl-benzenesulfonylamino)-5,6,7,8-tetrahydro-naphthalen-1-yloxy]-acetic acid;
[(R)-5-(3-fluoro-5-trifluoromethyl-benzenesulfonylamino)-5,6,7,8-tetrahydro-naphthalen-1-yloxy]-acetic acid; and
any pharmaceutically acceptable salt or ester thereof.

In another specific embodiment, the present invention is directed to a compound of formula I selected from the group consisting of:
{(R)-5-[(3-fluoro-5-trifluoromethyl-benzenesulfonyl)-methyl-amino]-5,6,7,8-tetrahydro-naphthalen-1-yloxy}-acetic acid;
{(R)-5-[(3,5-di-tert-butyl-benzenesulfonyl)-methyl-amino]-5,6,7,8-tetrahydro-naphthalen-1-yloxy}-acetic acid;
{(R)-5-[(3,5-bis-methanesulfonyl-benzenesulfonyl)-methyl-amino]-5,6,7,8-tetrahydro-naphthalen-1-yloxy}-acetic acid;
{(R)-5-[(3-methoxy-5-trifluoromethyl-benzenesulfonyl)-methyl-amino]-5,6,7,8-tetrahydro-naphthalen-1-yloxy}-acetic acid;
{(R)-5-[(3-bromo-5-trifluoromethyl-benzenesulfonyl)-methyl-amino]-5,6,7,8-tetrahydro-naphthalen-1-yloxy}-acetic acid;
{(R)-5-[(3,5-bis-trifluoromethyl-benzenesulfonyl)-methyl-amino]-5,6,7,8-tetrahydro-naphthalen-1-yloxy}-acetic acid;
{(R)-5-[(3,5-dichloro-benzenesulfonyl)-methyl-amino]-5,6,7,8-tetrahydro-naphthalen-1-yloxy}-acetic acid;
{(R)-5-[(3,5-difluoro-benzenesulfonyl)-methyl-amino]-5,6,7,8-tetrahydro-naphthalen-1-yloxy}-acetic acid;
{(R)-5-[(3,5-dimethyl-benzenesulfonyl)-methyl-amino]-5,6,7,8-tetrahydro-naphthalen-1-yloxy}-acetic acid;
((R)-5-{methyl-[3-(propane-2-sulfinyl)-5-trifluoromethyl-benzenesulfonyl]-amino}-5,6,7,8-tetrahydro-naphthalen-1-yloxy)-acetic acid;
{(R)-5-[(3-cyclopentanesulfonyl-5-trifluoromethyl-benzenesulfonyl)-methyl-amino]-5,6,7,8-tetrahydro-naphthalen-1-yloxy}-acetic acid;
((R)-5-{methyl-[3-(propane-2-sulfonyl)-5-trifluoromethyl-benzenesulfonyl]-amino}-5,6,7,8-tetrahydro-naphthalen-1-yloxy)-acetic acid;
((R)-5-{methyl-[3-(2-methyl-propane-2-sulfonyl)-5-trifluoromethyl-benzenesulfonyl]-amino}-5,6,7,8-tetrahydro-naphthalen-1-yloxy)-acetic acid;
{(R)-5-[methyl-(3-pyrrolidin-1-yl-5-trifluoromethyl-benzenesulfonyl)-amino]-5,6,7,8-tetrahydro-naphthalen-1-yloxy}-acetic acid;
{(R)-5-[(3-diethylamino-5-trifluoromethyl-benzenesulfonyl)-methyl-amino]-5,6,7,8-tetrahydro-naphthalen-1-yloxy}-acetic acid;
((R)-5-{[3-(isopropyl-methyl-amino)-5-trifluoromethyl-benzenesulfonyl]-methyl-amino}-5,6,7,8-tetrahydro-naphthalen-1-yloxy)-acetic acid;
((R)-5-{[3-(1,1-difluoro-ethyl)-5-trifluoromethyl-benzenesulfonyl]-methyl -amino}-5,6,7,8-tetrahydro-naphthalen-1-yloxy)-acetic acid;
{(R)-5-[(3-acetyl-5-trifluoromethyl-benzenesulfonyl)-methyl-amino]-5,6,7,8-tetrahydro-naphthalen-1-yloxy}-acetic acid;
((R)-5-{methyl-[3-(1-methyl-cyclopropyl)-5-trifluoromethyl-benzenesulfonyl]-amino}-5,6,7,8-tetrahydro-naphthalen-1-yloxy)-acetic acid;
{(R)-5-[(3-isopropyl-5-trifluoromethyl-benzenesulfonyl)-methyl-amino]-5,6,7,8-tetrahydro-naphthalen-1-yloxy}-acetic acid;
{(R)-5-[(3-isopropoxy-5-trifluoromethyl-benzenesulfonyl)-methyl-amino]-5,6,7,8-tetrahydro-naphthalen-1-yloxy}-acetic acid;
{(R)-5-[(3-ethoxy-5-trifluoromethyl-benzenesulfonyl)-methyl-amino]-5,6,7,8-tetrahydro-naphthalen-1-yloxy}-acetic acid;
{(R)-5-[(3-cyclopentyloxy-5-trifluoromethyl-benzenesulfonyl)-methyl-amino]-5,6,7,8-tetrahydro-naphthalen-1-yloxy}-acetic acid; and
any pharmaceutically acceptable salt or ester thereof.

In another particular embodiment, the present invention is directed to the compounds of formula I or formula IA or pharmaceutically acceptable salts or esters thereof except for [(R)-5-(3-fluoro-5-trifluoromethyl-benzenesulfonylamino)-5,6,7,8-tetrahydro-naphthalen-1-yloxy]-acetic acid and/or {(R)-5-[(3-fluoro-5-trifluoromethyl-benzenesulfonyl)-methyl-amino]-5,6,7,8-tetrahydro-naphthalen-1-yloxy}-acetic acid; and/or any pharmaceutically acceptable salt or ester thereof.

In another particular embodiment, the present invention is directed to the compounds of formula I or formula IA or pharmaceutically acceptable salts or esters thereof except for [(R)-5-(3,5-difluoro-benzenesulfonylamino)-5,6,7,8-tetrahydro-naphthalen-1-yloxy]-acetic acid and/or {(R)-5-[(3,5-difluoro-benzenesulfonyl)-methyl-amino]-5,6,7,8-tetrahydro-naphthalen-1-yloxy}-acetic acid; and/or any pharmaceutically acceptable salt or ester thereof.

In another particular embodiment, the present invention is directed to the compounds of formula I or formula IA or pharmaceutically acceptable salts or esters thereof except for [(R)-5-(3,5-dichloro-benzenesulfonylamino)-5,6,7,8-tetrahydro-naphthalen-1-yloxy]-acetic acid and/or {(R)-5-[(3,5-dichloro-benzenesulfonyl)-methyl-amino]-5,6,7,8-tetrahydro-naphthalen-1-yloxy}-acetic acid; and/or any pharmaceutically acceptable salt or ester thereof.

In another particular embodiment, the present invention is directed to the compounds of formula I or formula IA or pharmaceutically acceptable salts or esters thereof except for [(R)-5-(3,5-dimethyl-benzenesulfonylamino)-5,6,7,8-tetrahydro-naphthalen-1-yloxy]-acetic acid and/or {(R)-5-[(3,5-dimethyl-benzenesulfonyl)-methyl-amino]-5,6,7,8-tetrahydro-naphthalen-1-yloxy}-acetic acid; and/or any pharmaceutically acceptable salt or ester thereof.

In another particular embodiment, the present invention is directed to the compounds of formula I or formula IA or pharmaceutically acceptable salts or esters thereof except for [(R)-5-(3-diethylamino-5-trifluoromethyl-benzenesulfonylamino)-5,6,7,8-tetrahydro-naphthalen-1-yloxy]-acetic acid and/or {(R)-5-[(3-diethylamino-5-trifluoromethyl-benzenesulfonyl)-methyl-amino]-5,6,7,8-tetrahydro-naphthalen-1-yloxy}-acetic acid; and/or any pharmaceutically acceptable salt or ester thereof.

In another particular embodiment, the present invention is directed to the compounds of formula I or formula IA or pharmaceutically acceptable salts or esters thereof except for [(R)-5-(3-cyclopentyloxy-5-trifluoromethyl-benzenesulfonylamino)-5,6,7,8-tetrahydro-naphthalen-1-yloxy]-acetic acid and/or {(R)-5-[(3-cyclopentyloxy-5-trifluoromethyl-benzenesulfonyl)-methyl-amino]-5,6,7,8-tetrahydro-naphthalen-1-yloxy}-acetic acid; and/or any pharmaceutically acceptable salt or ester thereof.

### General Synthesis of Compounds According to the Invention

The compounds of the present invention can be prepared by any conventional means. Suitable processes for synthesizing these compounds are provided in the examples. Generally, compounds of formula I can be prepared according to the schemes illustrated below.

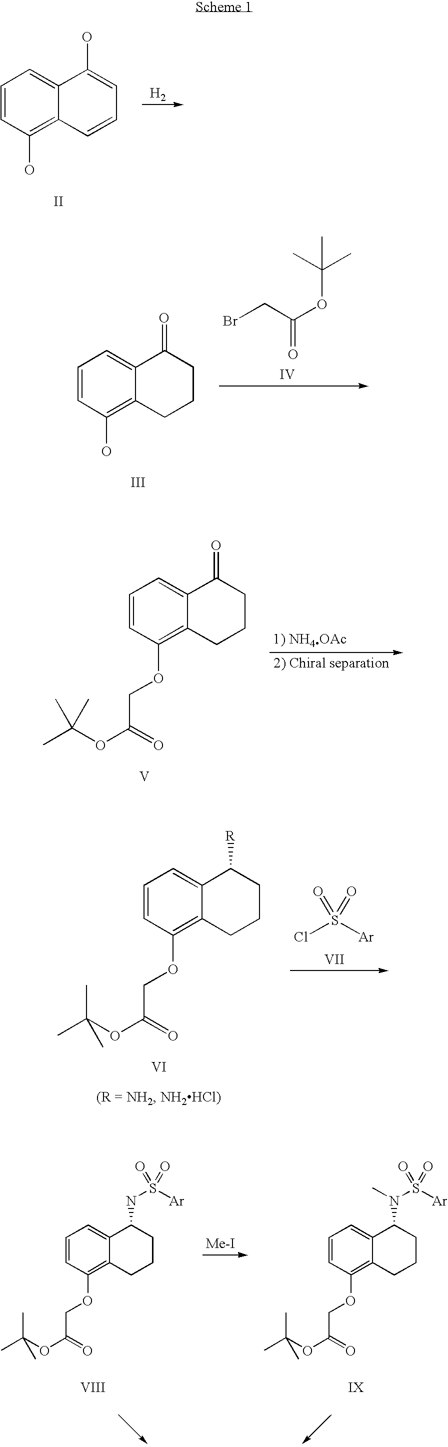

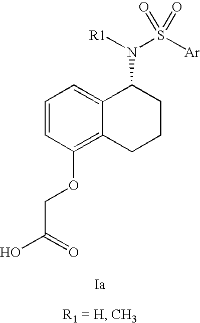

Compounds of interest la can be prepared according to Scheme 1. Starting with naphthalene-1,5-diol (II), palladium catalyzed hydrogenation gives 5-hydroxy-3,4-dihydro-2H-naphthalen-1-one (III), which undergoes nucleophilic substitution with tert-butyl bromoacetate (IV) under basic conditions to generate the ether compound V. Reductive amination of the intermediate V with ammonium acetate followed by chiral separation yields the corresponding amino derivative VI. Sulfonylation of amine VI (or its hydrochloride salt) with a variety of aryl sulfonyl chlorides VII affords sulfonamides of structures VIII. N-Methylation of the N—H sulfonamides VIII gives compounds IX. Ester hydrolysis of either VIII or IX produces compounds of interest Ia. It is also possible to synthesize enantiomerically pure compounds of interest Ia, starting with racemic VI (or its hydrochloride salt), and using a subsequent chiral resolution of racemic intermediates VIII or IX. Alternatively, optically pure Ia can be obtained via a chiral separation of racemic compounds of interest Ia.

5-Hydroxy-3,4-dihydro-2H-naphthalen-1-one (III), which is commercially available, can be prepared by hydrogenation of naphthalene-1,5-diol (II). The reaction can be carried out in the presence of palladium on carbon (10%) under 100 psi pressure of hydrogen under basic conditions in a solvent such as isopropanol, ethanol, ethyl acetate, or methanol, at 80° C. for several hours.

The nucleophilic substitution reaction of 5-hydroxy-3,4-dihydro-2H-naphthalen-1-one (III) with tert-butyl bromoacetate (IV) to give the ether compound V can be accomplished using methods that are well known to someone skilled in the art. The reaction is typically carried out in the presence of a carbonate base (e.g. cesium carbonate, potassium carbonate, or the like) or potassium hydroxide in an aprotic solvent such as acetonitrile, N,N-dimethylformamide, or dimethyl sulfoxide, at a temperature between 50 and 100° C. for several hours.

Transformation of ketone V to amine VI can be achieved via reductive amination. The conversion can be carried out in stepwise fashion by treating ketone V with ammonium acetate or ammonia to generate the corresponding imine, which can then be isolated and reduced with a suitable reducing agent (e.g. sodium borohydride). It is also possible to carry out the same reaction sequence in one pot, with the imine formation and reduction occurring concurrently with the use of reducing agents such as sodium cyanoborohydride (NaBH₃CN) or sodium triacetoxyborohydride (NaBH(OCOCH₃)₃). The reaction is typically done in a solvent such as methanol or tetrahydrofuran, at a temperature between room temperature and reflux temperature for several hours. Chiral chromatography is then used to separate the enantiomers of the racemic amine thus obtained to afford the optically pure R enantiomer of amine VI.

Sulfonylation of the amine compound VI (or its hydrochloride salt) with the aryl sulfonyl chlorides of structures VII to give sulfonamides VIII can be easily accomplished using methods well known to someone skilled in the art. The reaction is typically carried out in the presence of a base such as triethylamine, diisopropylethylamine, pyridine, or dimethyl-pyridin-4-yl-amine in a suitable inert solvent such as dichloromethane, acetonitrile, 1,4-dioxane, tetrahydrofuran or mixtures thereof, at room temperature for 16 hours.

N-Methylation of compounds VIII to produce the corresponding derivatives IX can be achieved by treating compounds VIII with methyl iodide in the presence of a weak base such as potassium carbonate or sodium carbonate, in an inert solvent such as N,N-dimethylformamide, acetonitrile, or tetrahydrofuran, at 65° C. for 5 hours.

Hydrolysis of compounds VIII or IX gives the acids Ia. The reaction can be carried out in the presence of an aqueous inorganic base such as sodium hydroxide or potassium hydroxide, in an inert solvent such as 1,4-dioxane or tetrahydrofuran, at room temperature for several hours.

Alternatively, the optically pure enantiomers of compounds of interest Ia can be obtained via the same route as described above starting with the racemic amine precursor for VI (Scheme 1, step 3, prior to resolution), and using a later step chiral separation of the racemic compounds corresponding to VIII, IX or Ia.

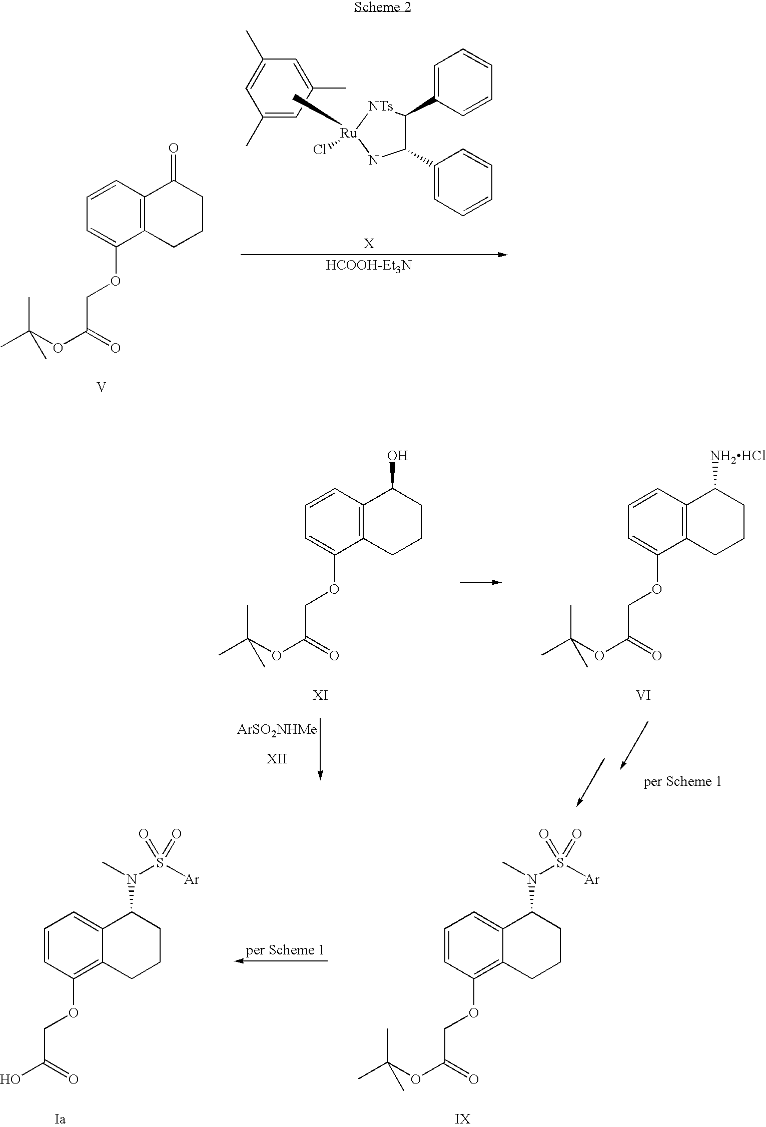

Alternatively, the key chiral intermediate VI can be prepared via an asymmetric synthesis approach shown in Scheme 2. Reduction of the ketone V to the corresponding hydroxyl compound XI can be done enantioselectively by using the chiral catalyst of formula X (or a similar catalyst containing cymene in place of mesitylene) in the presence of formic acid-triethylamine azeotropes. The hydroxyl compound XI is then converted to the amine hydrochloride salt VI via a two step process: First, the alcohol XI is converted to the corresponding azido analogue (with high preference for inversion of stereochemistry) using diphenylphosphoryl azide (DPPA) and 1,8-diazabicyclo[5.4.0]undec-7-ene (DBU). Hydrogenation of the azido derivative, followed by treatment with chlorotrimethylsilane and methanol, gives the amine hydrochloride VI bearing the desired stereochemistry. The key intermediate VI can then be converted to intermediates IX, and subsequently transformed to compounds of interest la, as previously described in Scheme 1.

Additionally, the chiral alcohol XI can be converted to the key sulfonamide intermediates IX via a one-step Mitsunobu reaction with the appropriate sulfonamides XII. Ester hydrolysis to produce compounds of interest la can then be carried out as previously described in Scheme 1.

Reduction of the ketone V to the hydroxyl compound XI can be done enantioselectively by using a catalyst such as chloro-[(1S,2S)-N-(p-toluenesulfonyl)-1,2-diphenylethane-diamine] (mesitylene) ruthenium(II) (X), or a similar catalyst containing cymene in place of mesitylene, in formic acid-triethylamine azeotropes (5:2 molar ratio) at room temperature for several hours, and then at 45° C. for another few hours (references: Fujii, A. et al., J. Am. Chem. Soc. 118 (1996) 2521; Wagner, K. Angew. Chem., Int. Ed. Engl. 9 (1970), 50).

Displacement of the hydroxyl group of structure XI to give the corresponding azido analogue (with a high selectivity for inversion of stereochemistry) can be achieved by treating a mixture of compound XI and diphenylphosphoryl azide (DPPA) with 1,8-diazabicyclo[5.4.0]undec-7-ene (DBU) under anhydrous conditions at a temperature between 0° C. and 10° C. for 18 hours in an inert solvent such as toluene or N,N-dimethylformamide.

Hydrogenation of the above azido derivative to give the corresponding amine VI with retained chirality can be carried out in the presence of 5% palladium on carbon under 350 psi pressure of hydrogen, at room temperature for 1.5 hour, in an organic solvent such as ethyl acetate, methanol, or ethanol.

The Mitsunobu reaction between the alcohol derivative XI and the sulfonamides XII is well known to someone skilled in the art. The reaction is typically carried out in the presence of triphenylphosphine and diisopropyl azodicarboxylate, in a solvent such as tetrahydrofuran, or 2-methyl-tetrahydro-furan, at a temperature between −10° C. and −20° C.

The conversion of key intermediates VI or IX to the compounds of interest la is then carried as previously described in Scheme 1 above.

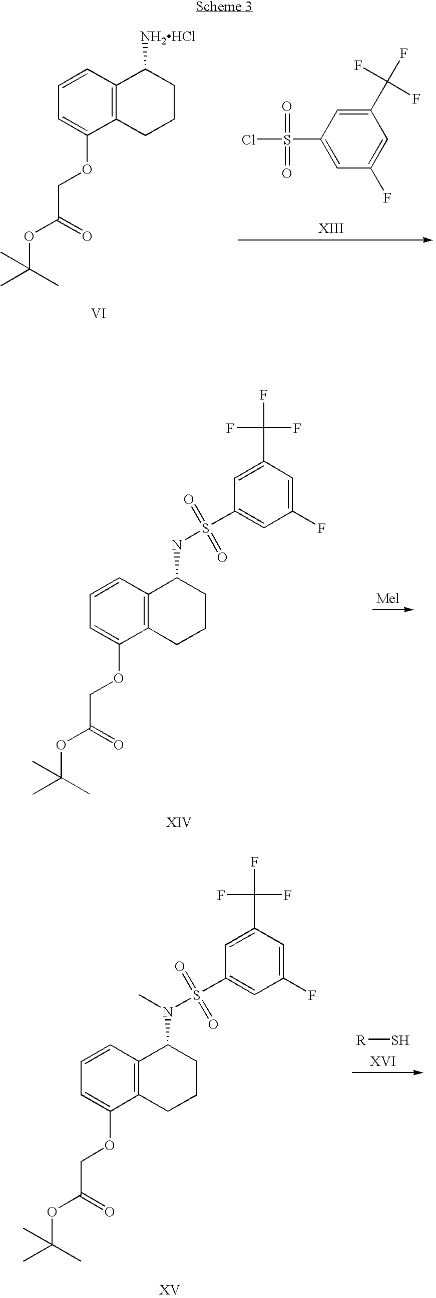

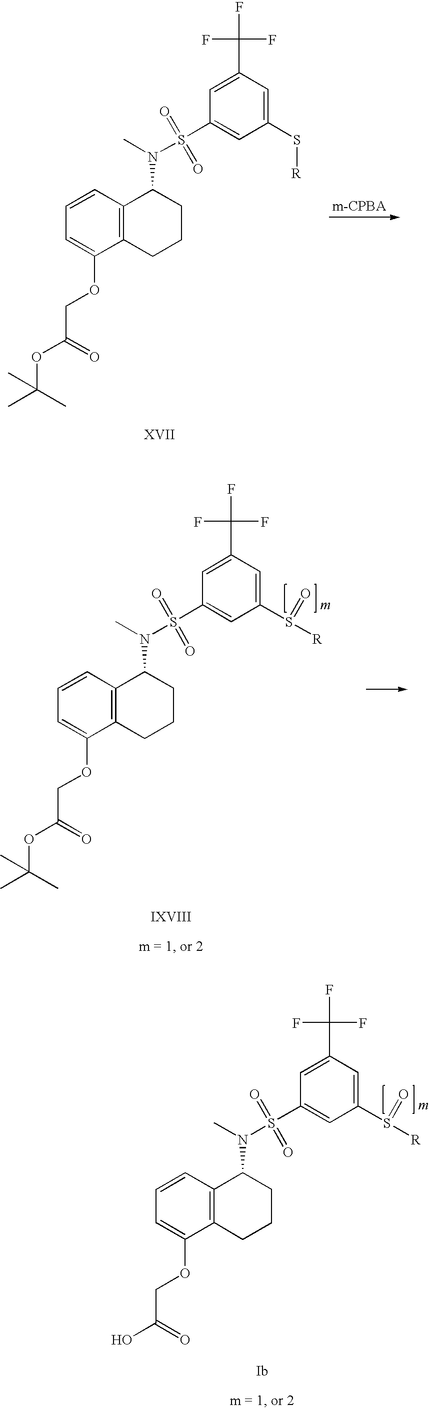

Compounds of interest Ib, with sulfonyl or sulfinyl groups on the aryl sulfonamides, can be prepared according to Scheme 3. A sulfonylation reaction of ((R)-5-amino-5,6,7,8-tetrahydro-napthalen-1-yloxy)-acetic acid tert-butyl ester hydrochloride salt (VI) and 3-fluoro-5-trifluoromethyl-benzenesulfonyl chloride (XIII) gives compound XIV, which upon methylation is converted to the corresponding N-methylated derivative XV. Nucleophilic substitution of the intermediate XV with thiols XVI affords the sulfanyl compounds XVII, which can be transformed to either sulfinyl (m=1) or sulfonyl (m=2) derivatives XVIII via oxidation under controlled conditions. Ester hydrolysis of XVIII produces compounds of interest Ib.

Sulfonylation of the amine hydrochloride salt VI with 3-fluoro-5-trifluoromethyl-benzenesulfonyl chloride (XIII) to give sulfonamides XIV can be easily accomplished using methods well known to someone skilled in the art. The reaction is typically carried out in the presence of a base such as triethylamine, diisopropylethylamine, pyridine, or dimethyl-pyridin-4-yl-amine in a suitable inert solvent such as dichloromethane, acetonitrile, 1,4-dioxane, tetrahydrofuran or mixtures thereof, at room temperature for 16 hours.

N-Methylation of N—H compound XIV to produce the derivatives XV can be achieved by treating compound XIV with methyl iodide in the presence of a weak base such as potassium carbonate or sodium carbonate, in an inert solvent such as N,N-dimethylformamide, acetonitrile, or tetrahydrofuran, at 65° C. for 5 hours.

Nucleophilic substitution of the fluoro-substituted compound XV with thiols XVI to give the 3-alkylsulfanyl analogues XVII can be done in the presence of a base, such as potassium carbonate, cesium carbonate, sodium acetate, or triethylamine, in a solvent such as N,N-dimethylformamide, dimethyl sulfoxide, ethanol, water or mixtures thereof, at a temperature between 100 and 150° C. for about 30 to 60 minutes under microwave irradiation. Alternatively, the reaction can be also carried out without the use of a microwave at a moderately elevated temperature for a longer reaction time.

Oxidation of the sulfanyl compounds XVII to the corresponding sulfinyl or sulfonyl analogues XVIII can be achieved using an oxidant such as hydrogen peroxide or m-chloroperoxybenzoic acid (m-CPBA), in an inert suitable solvent such as dichloromethane or dichloroethane (or an aqueous solution if hydrogen peroxide is used), at a temperature between 0° C. and room temperature for several hours. Alternatively, OXONE/alumina can be used under controlled conditions to give either sulfoxides or sulfones XVIII. Typically, the reaction is carried out in a suitable solvent such as ethanol, methanol, acetone, dichloromethane, water or mixture thereof, at the temperature between 0° C. and reflux temperature for several hours. Formation of sulfoxide or sulfone relies on the stoichiometry of the reaction and reaction time. (reference: Llauger L., et al., Tetrahedron Lett. 45 (2004) 9549-9552; Kropp P. J., et al., J. Am. Chem. Soc., 122 (2000), 4280-4285).

Hydrolysis of esters XVIII gives the compounds of interest of formula Ib. The reaction can be carried out in the presence of an aqueous inorganic base such as sodium hydroxide or potassium hydroxide, in an inert solvent such as 1,4-dioxane or tetrahydrofuran, at room temperature for several hours.

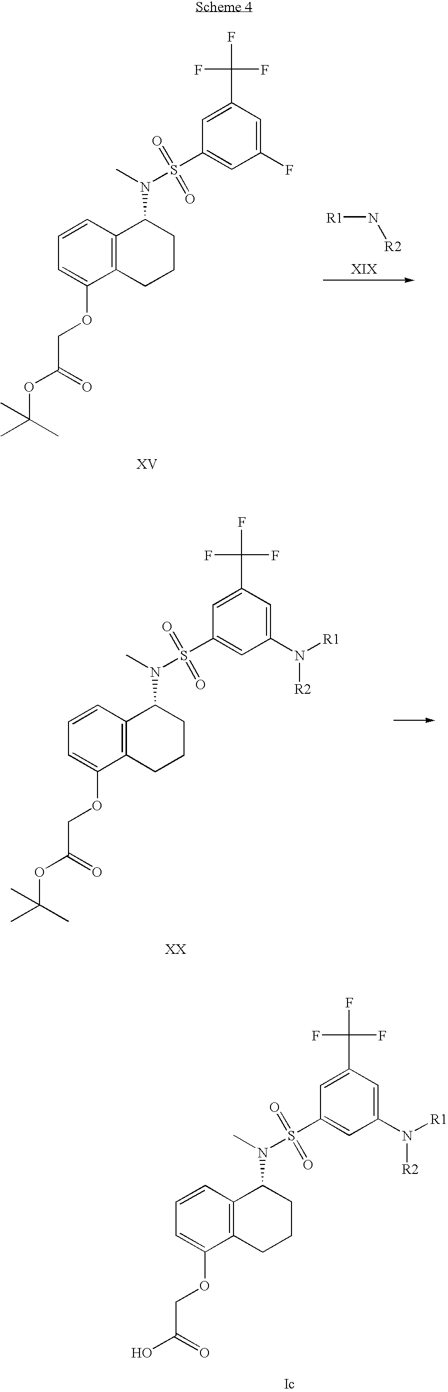

Compounds of interest Ic can be prepared according to Scheme 4, by nucleophilic substitution reactions of the corresponding fluoro substituted aryl sulfonamide XV with the appropriate amines XIX to give the amino-substituted intermediates XX, followed by base-catalyzed ester hydrolysis.

The nucleophilic substitution of the fluoro group of compound XV with various amines XIX to generate the amino derivatives XX can be carried out with or without the presence of a base such as sodium hydride, potassium carbonate, or cesium carbonate, in an inert solvent such as tetrahydrofuran, dimethyl sulfoxide, or N,N-dimethylformamide at a temperature between 100 and 150° C. for 15 to 60 minutes under microwave irradiation. Alternatively, the reactions can be performed at an elevated temperature for a longer reaction time without microwave irradiation.

Hydrolysis of esters XX gives the compounds of interest of formula Ic. The reactions can be carried out in the presence of an aqueous inorganic base such as sodium hydroxide or potassium hydroxide, in an inert solvent such as 1,4-dioxane or tetrahydrofuran, at room temperature for several hours.

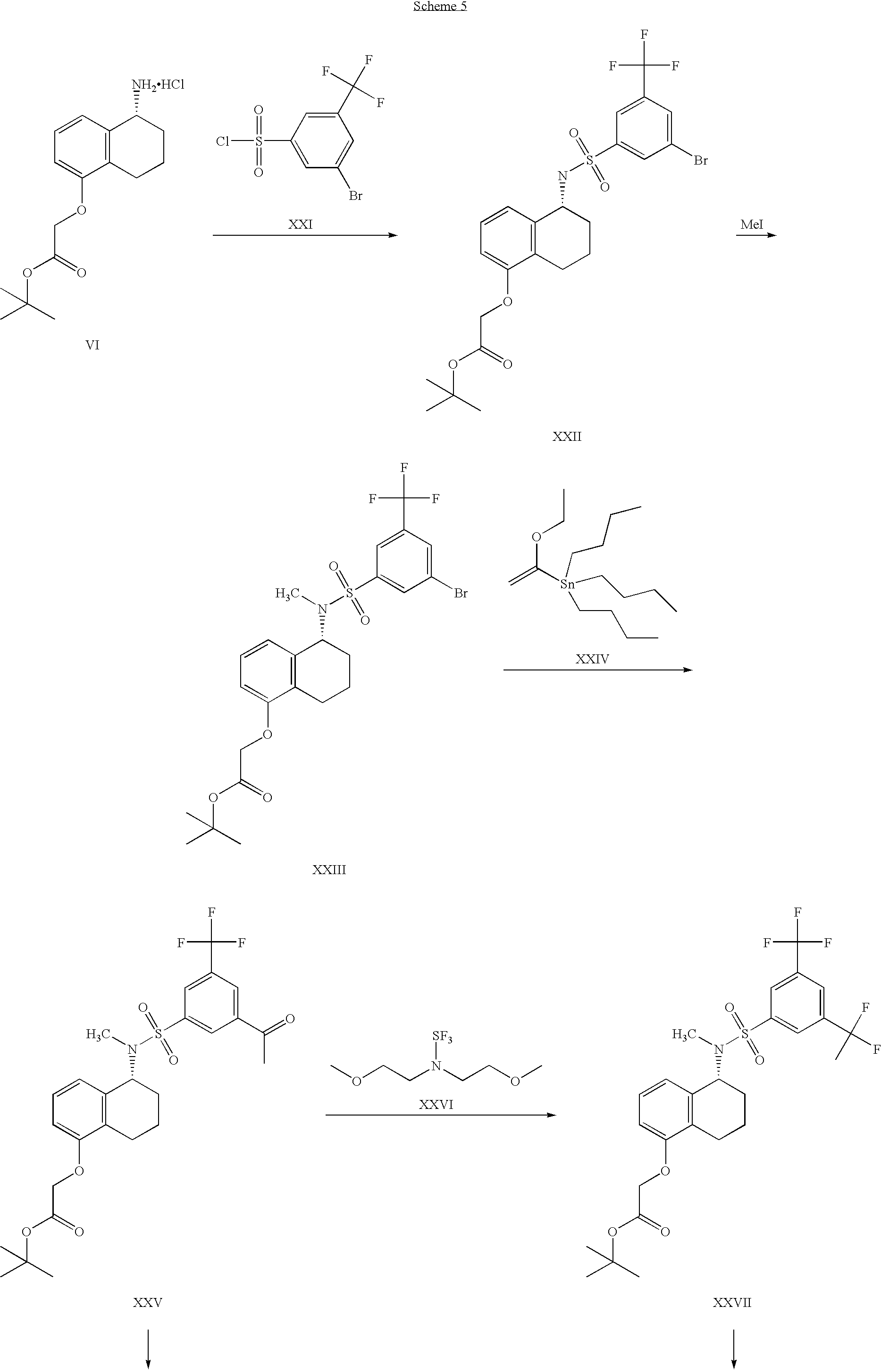

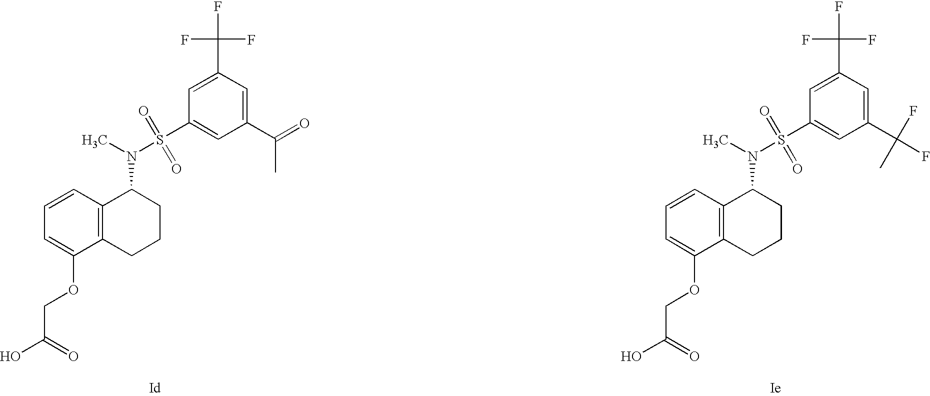

Synthesis of the compounds of interest Id and Ie is illustrated in Scheme 5. Sulfonylation of the amine hydrochloride salt VI with the bromo-substituted aryl sulfonyl chloride XXI gives the corresponding sulfonamide XXII. The sulfonamide N—H in XXII can be substituted with a methyl group to give the corresponding derivative XXIII. A Stille coupling reaction between the aryl bromide XXIII and tributyl(1-ethoxyvinyl)stannane (XXIV), followed by acidic workup, produces the ketone XXV, which can then be transformed to the gem-difluoride XXVII upon treatment with nucleophilic fluorinating sources. Ester hydrolysis of the methyl sulfonamides XXV or XXVII generates the compounds of interest Id and Ie, respectively.

Sulfonylation of the amine hydrochloride salt VI with 3-bromo-5-trifluoromethyl-benzenesulfonyl chloride (XXI) to give the sulfonamide XXII can be easily accomplished using methods well known to someone skilled in the art. The reaction is typically carried out in the presence of a base such as triethylamine, diisopropylethylamine, pyridine, or dimethyl-pyridin-4-yl-amine in a suitable inert solvent such as dichloromethane, acetonitrile, 1,4-dioxane, tetrahydrofuran or mixtures thereof, at room temperature for 16 hours.

N-Methylation of sulfonamide XXII to produce the corresponding derivative XXIII can be achieved by treating XXII with methyl iodide in the presence of a weak base such as potassium carbonate or sodium carbonate, in an inert solvent such as N,N-dimethylformamide, acetonitrile, or tetrahydrofuran, at a temperature around 70° C. for several hours.

The ketone XXV can be obtained by the Stille coupling reaction between the bromo derivative XXIII and tributyl(1-ethoxyvinyl)stannane (XXIV), followed by acidic hydrolysis with hydrochloric acid at room temperature to 70° C. for 30 minutes to 18 hours in water or a mixture of water and tetrahydrofuran. The Stille coupling reaction is typically carried out in the presence of a palladium catalyst such as tetrakis(triphenylphosphine)palladium(0) (Pd(PPh₃)₄) or [1,1′-bis(diphenylphosphino)ferrocene]dichloropalladium(II) (PdCl₂(dppf)), in an inert solvent such as N,N-dimethylformamide, toluene, dioxane, acetonitrile, or mixtures thereof, at a temperature between 80 and 150° C. for 1 to 18 hours under an argon atmosphere. Alternatively, the reaction can be carried out in the presence of tris(dibenzylideneacetone)dipalladium(0) (Pd₂(dba)₃), and triphenylarsine (Ph₃As).

Transformation of ketone XXV to the gem-difluoride derivatives XXVII can be accomplished with nucleophilic fluorinating sources such as diethylaminosulfur trifluoride (DAST), bis(2-methoxyethyl)aminosulfur trifluoride, (CH₃OCH₂CH₂)₂NSF₃ (Deoxo-Fluor reagent), α,α-difluoroamines, or N,N-diethyl-α,α-difluoro-(m-methylbenzyl)amine (DFMBA), either with or without a suitable solvent such as tetrahydrofuran, dichloromethane, or mixtures thereof, at a temperature between room temperature and 180° C. for several hours (reference: Lal, G. S. et al., J. Org. Chem. 64 (1999) 7048).

Ester hydrolysis reactions of either XXV or XXVII produce the compounds of interest of formula Id and Ie, respectively. The reaction can be carried out in the presence of an aqueous inorganic base such as sodium hydroxide or potassium hydroxide, in an inert solvent such as 1,4-dioxane or tetrahydrofuran, at room temperature for several hours.

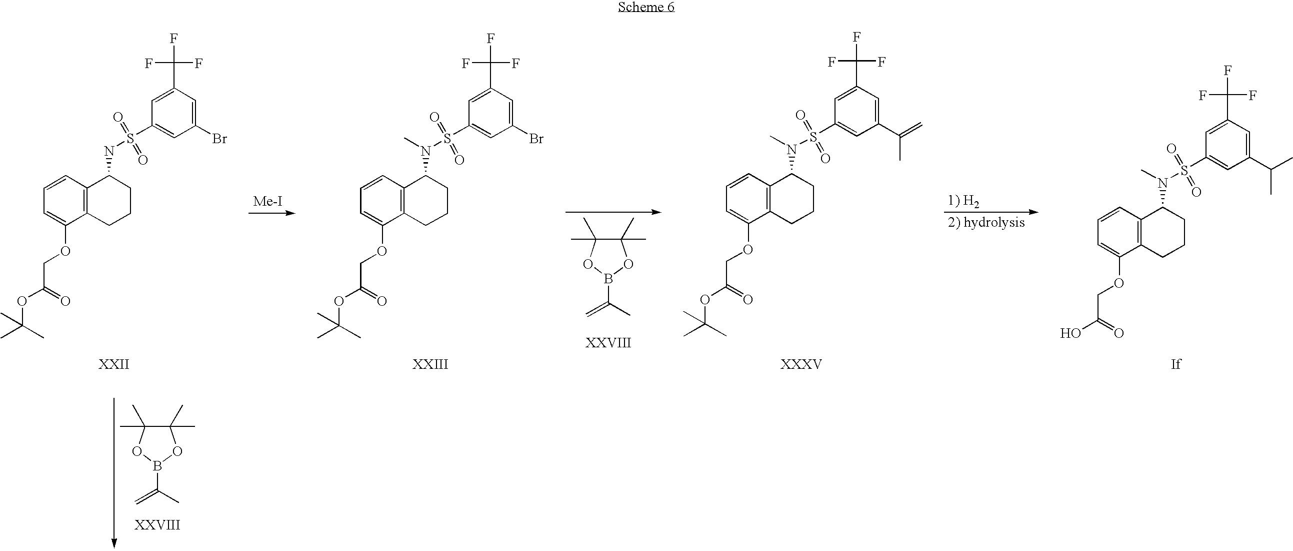

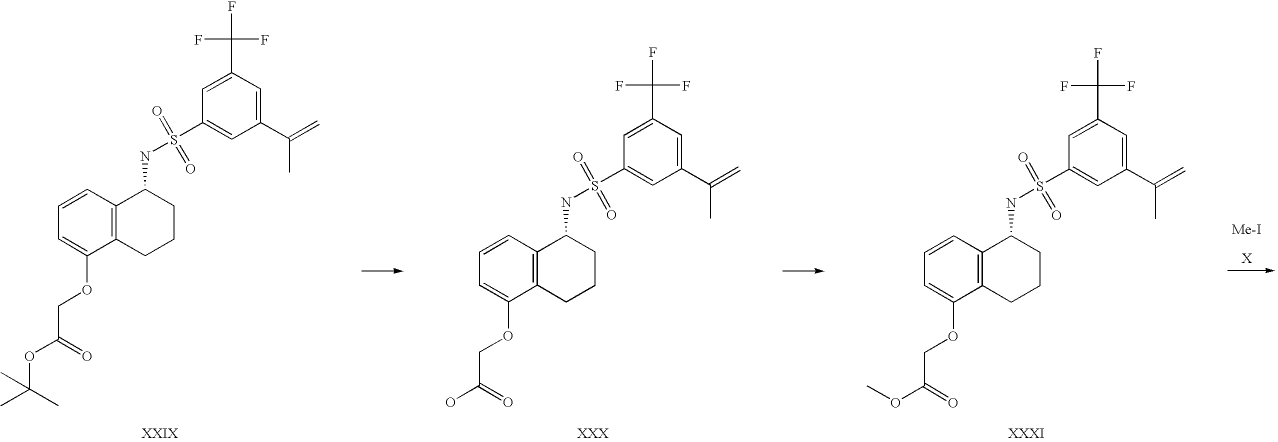

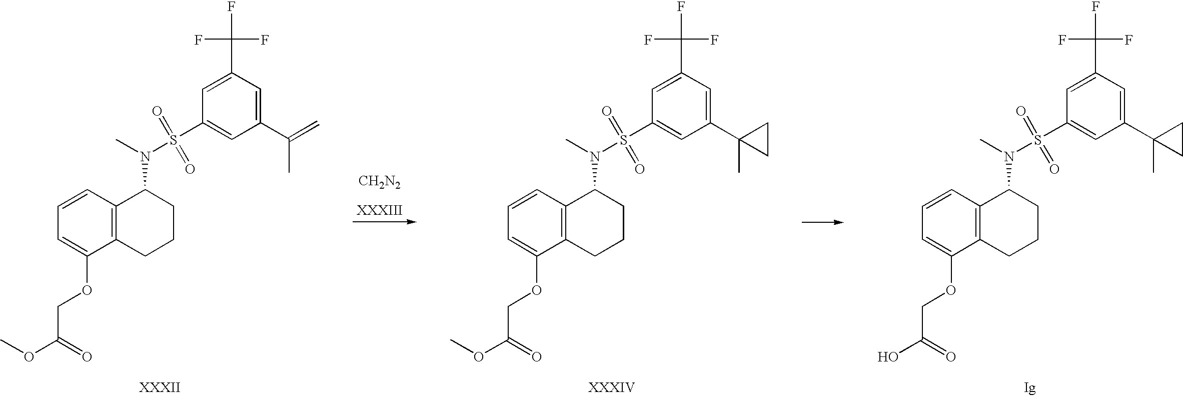

Compounds of interest If and Ig can be synthesized as illustrated in Scheme 6. Suzuki coupling reaction between the bromo-substituted compound XXII and 2-isopropenyl-4,4,5,5-tetramethyl-[1,3,2]dioxaborolane (XXVIII) generates the corresponding isopropenyl compound of structure XXIX. Ester hydrolysis of tert-butyl ester XXIX, followed by re-esterification with methanol gives the methyl ester XXXI, which is further N-methylated to yield intermediate XXXII. Treatment of olefin XXXII with diazomethane (XXXIII) followed by ester hydrolysis produces the compound of interest Ig. N-Methylation of the bromo-substituted compound XXII gives the corresponding derivative XXIII, which is then transformed to the N-methylated olefin XXXV via Suzuki coupling reaction with 2-isopropenyl-4,4,5,5-tetramethyl-[1,3,2]dioxaborolane (XXVIII). Hydrogenation of the olefin XXXV, and subsequent ester hydrolysis affords compound If.

The Suzuki coupling reaction between compound XXII and 2-isopropenyl-4,4,5,5-tetramethyl-[1,3,2]dioxaborolane (XXVIII) to give the olefin derivative XXIX can be carried out in the presence of a palladium catalyst such as tetrakis(triphenylphosphine)palladium(0) (Pd(PPh₃)₄), or [1,1′-bis(diphenylphosphino)ferrocene]dichloropalladium(lI) (PdCl₂(dppf)), and a base such as potassium tert-butoxide or sodium carbonate, in an inert solvent such as N,N-dimethylformamide or dimethyl sulfoxide, at a temperature between 130 and 180° C. for 15 to 30 minutes under microwave irradiation. Alternatively, the reaction can be carried out without the use of a microwave at a heated temperature such as 130° C. for a longer reaction time.

Ester transformation of tert-butyl ester XXIX to the methyl ester XXXI can be accomplished in two steps. The first step involves a base-catalyzed hydrolysis of XXIX to the corresponding acid XXX. The reaction can be carried out in the presence of an aqueous inorganic base such as lithium hydroxide or potassium hydroxide, in an inert solvent such as 1,4-dioxane or tetrahydrofuran, at room temperature for several hours. The methyl ester XXXI can be obtained by treating the acid intermediate XXX in methanol in the presence of a catalytic amount of thionyl chloride under microwave irradiation at a temperature of about 100° C. for 15 to 30 minutes.

The corresponding N-methyl compound XXXII can be readily prepared by methylation of compound XXXI with methyl iodide (X). The reaction can be carried out in the presence of a weak base such potassium carbonate or sodium carbonate, in an inert solvent such as N,N-dimethylformamide, acetonitrile, or tetrahydrofuran, at 65° C. for 5 hours.

Transformation of the olefin XXXII to the corresponding cyclopropyl derivative XXXIV can be achieved by treating compound XXXII with diazomethane (XXXIII) in the presence of a palladium catalyst such as palladium acetate, palladium(II)acetylacetone, or palladium dichloride bis(benzonitrile), in a solvent such as dichloromethane, diethyl ether, tetrahydrofuran, or mixtures thereof, at a temperature between 0° C. and room temperature for several hours [reference: Staas, D. D. et al. Bioorg. Med. Chem. 14 (2006) 6900]. Diazomethane can be freshly prepared in situ and used in a solution of ether or dioxane. For example, diazomethane is liberated from a solution of N-nitroso-N-methylurea in diethyl ether by the addition of aqueous potassium hydroxide at low temperatures.

Ester hydrolysis of the cyclopropyl compound XXXIV gives compound of interest of formula Ig. The reaction can be carried out in the presence of an aqueous inorganic base such as lithium hydroxide or potassium hydroxide, in an inert solvent such as 1,4-dioxane or tetrahydrofuran, at room temperature for several hours.

As described in Scheme 5, N-methylation of sulfonamide XXII to produce the corresponding derivative XXIII can be achieved by treating XXII with methyl iodide in the presence of a weak base such as potassium carbonate or sodium carbonate, in an inert solvent such as N,N-dimethylformamide, acetonitrile, or tetrahydrofuran, at a temperature around 70° C. for several hours.

The Suzuki coupling reaction between the N-methylated compound XXIII and 2-isopropenyl-4,4,5,5-tetramethyl-[1,3,2]dioxaborolane (XXVIII) to give the olefin derivative XXXV can be carried out in similar fashion as described above, in the presence of a palladium catalyst such as tetrakis(triphenylphosphine)palladium(0) (Pd(PPh₃)₄), or [1,1′-bis(diphenylphosphino)ferrocene]dichloropalladium(lI) (PdCl₂(dppf)), and a base such as potassium tert-butoxide or sodium carbonate, in an inert solvent such as N,N-dimethylformamide or dimethyl sulfoxide, at a temperature between 130 and 180° C. for 15 to 30 minutes under microwave irradiation. Alternatively, the reaction can be carried out without the use of a microwave at a heated temperature such as 130° C. for a longer reaction time.

The compound of interest of formula If can be obtained through hydrogenation of intermediate XXXV, followed by ester hydrolysis. The hydrogenation can be carried out in the presence of 10% palladium on carbon under atmospheric pressure of hydrogen in a solvent such as ethanol, ethyl acetate, or methanol, at room temperature for several hours. Alternatively, the hydrogenation reaction can be carried out using a microwave in a solvent such as ethanol, ethyl acetate, or methanol, under a pressure of 50 psi, at 80° C. for several minutes. Ester hydrolysis can be accomplished in the presence of an aqueous inorganic base such as sodium hydroxide or potassium hydroxide, in an inert solvent such as 1,4-dioxane or tetrahydrofuran, at room temperature for several hours.

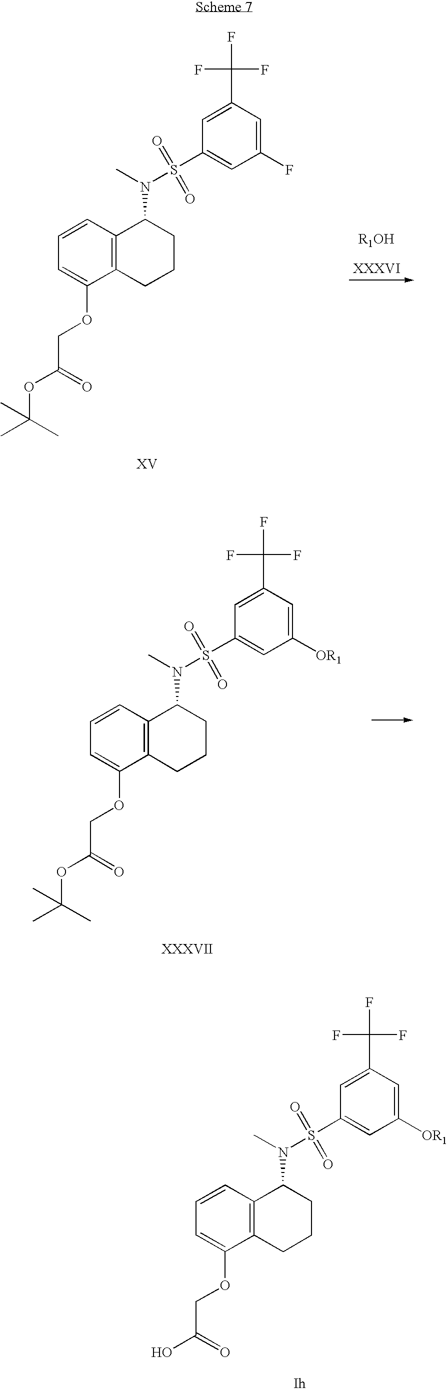

Compounds Ih, where an alkyl or cycloalkyl group (R₁) is linked to the aromatic ring through an ether linkage, can be prepared according to Scheme 7, by starting with nucleophilic substitution of the fluoro-substituted compound XV (prepared as described in Scheme 3) with the alkyl or cycloalkyl alcohols XXXVI to give the ethers XXXVII, followed by ester hydrolysis.

Conversion of the fluoro-substituted compound XV to ethers XXXVII can be achieved by nucleophilic substitution reactions with the appropriate alcohols XXXVI, in the presence of a base such as sodium hydride or potassium carbonate, in an inert solvent such as N,N-dimethylformamide at a temperature between 100 and 150° C. for 15 to 60 minutes under microwave irradiation.

Hydrolysis of compounds XXXVII gives the compounds of interest Ih. The reaction can be carried out in the presence of an aqueous inorganic base such as sodium hydroxide, lithium hydroxide, or potassium hydroxide, in an inert solvent such as tetrahydrofuran or 1,4-dioxane, at room temperature for several hours.

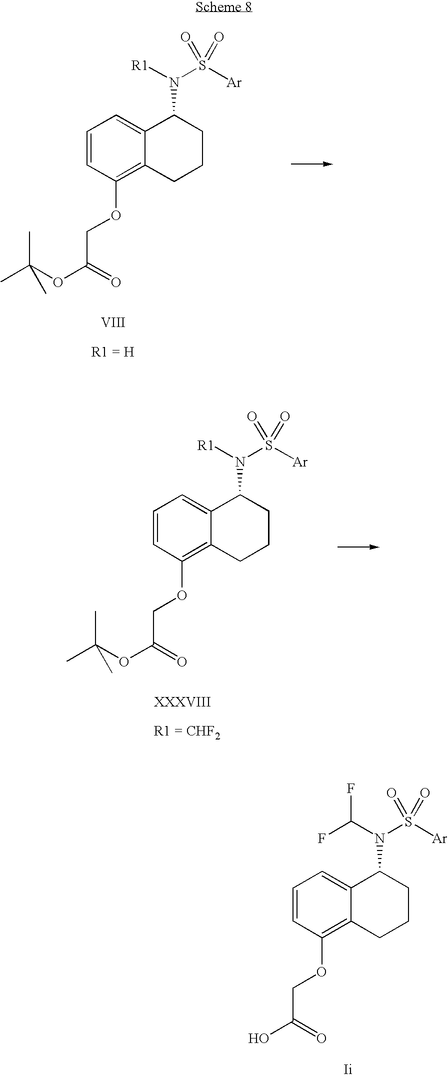

Compounds of interest Ii, which contain an N-difluoromethyl sulfonamide group, can be prepared as shown in Scheme 8 above. Derivatization of the N—H sulfonamides VIII (prepared as described in Scheme 1 above) gives intermediates XXXVIII. Ester hydrolysis of XXXVIII produces compounds of interest Ii.

Conversion of compounds VIII to the corresponding difluoromethyl sulfonamide derivatives XXXVIII can be achieved by treatment with chlorodifluoromethane (Freon-22) in the presence of a base such as potassium hydroxide, in an inert solvent such as N,N-dimethylformamide, acetonitrile, or tetrahydrofuran, at 70° C. for several hours [reference: Petko, K. et al, Russian Journal of Organic Chemistry, 38 (2002), 1030].

Hydrolysis of compounds XXXVIII gives the acids Ii. The reaction can be carried out in the presence of an aqueous inorganic base such as sodium hydroxide or potassium hydroxide, in an inert solvent such as 1,4-dioxane or tetrahydrofuran, at room temperature for several hours, or at 40° C. for 1 hour.

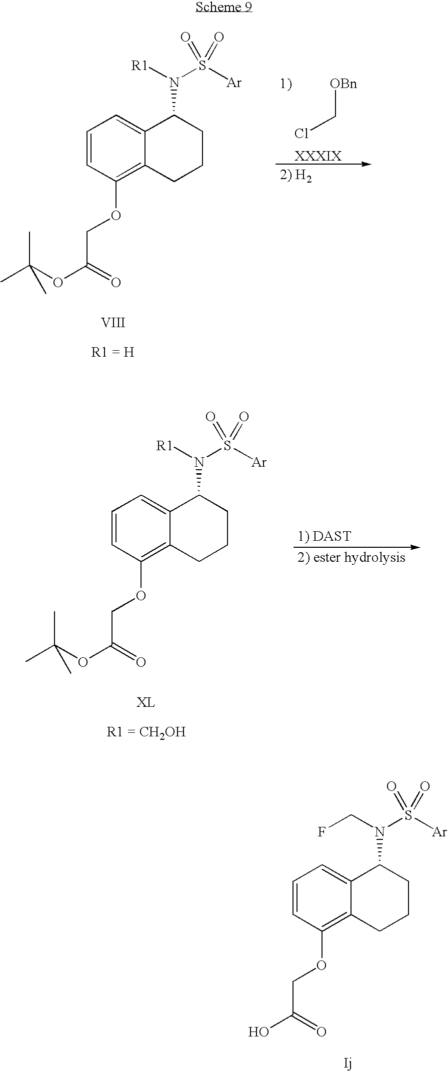

Compounds of interest Ij, which contain an N-fluoromethyl sulfonamide group, can be prepared as shown in Scheme 9 above. Derivatization of the N—H sulfonamides VIII (prepared as described in Scheme 1 above) via a two-step process gives the hydroxymethyl-substituted intermediates XL. Conversion of the hydroxymethyl derivatives XL to the corresponding fluoromethyl analogs, by treatment with diethylaminosulfur trifluoride (DAST), followed by ester hydrolysis, produces compounds of interest Ij.

Conversion of compounds VIII to the corresponding hydroxymethyl-substituted sulfonamide derivatives XL can be achieved by a two step process, as described by Rapoport, H. et al. [J. Org. Chem. 67 (2002) 1314]. Treatment of IX with benzyl chloromethyl ether, followed by hydrogenolysis of the resulting benzyl ether produces the hydroxymethyl-substituted derivatives XL.

Conversion of alcohols XL to the corresponding fluoromethyl-substituted derivatives can be accomplished by treatment with diethylaminosulfur trifluoride (DAST), as described by Beauve, C. et al. [Tetrahedron, 55 (1999) 13301] Hydrolysis of the resulting esters gives the acids Ii. The reaction can be carried out in the presence of an aqueous inorganic base such as sodium hydroxide or potassium hydroxide, in an inert solvent such as 1,4-dioxane or tetrahydrofuran, at room temperature for several hours, or at 40° C. for 1 hour.

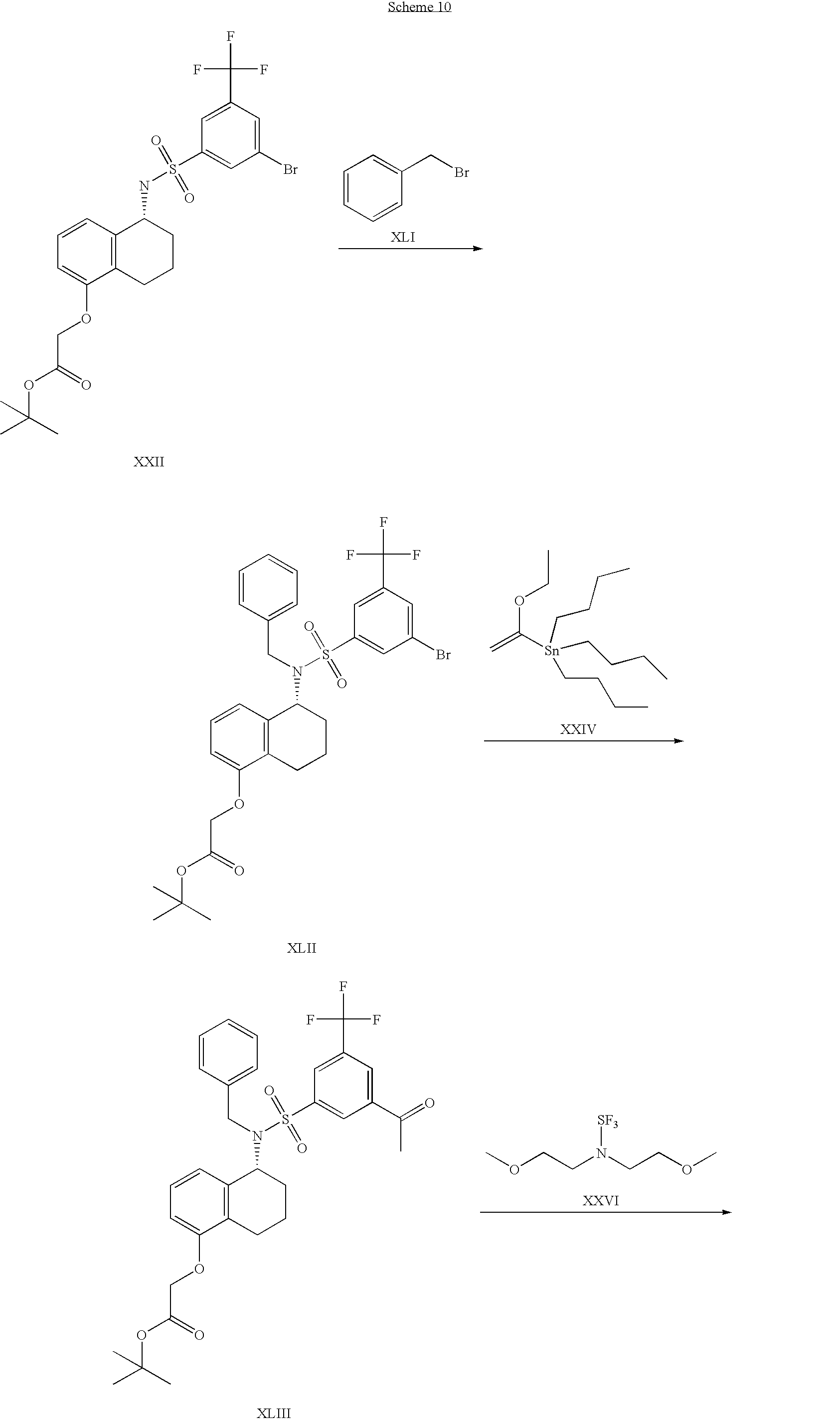

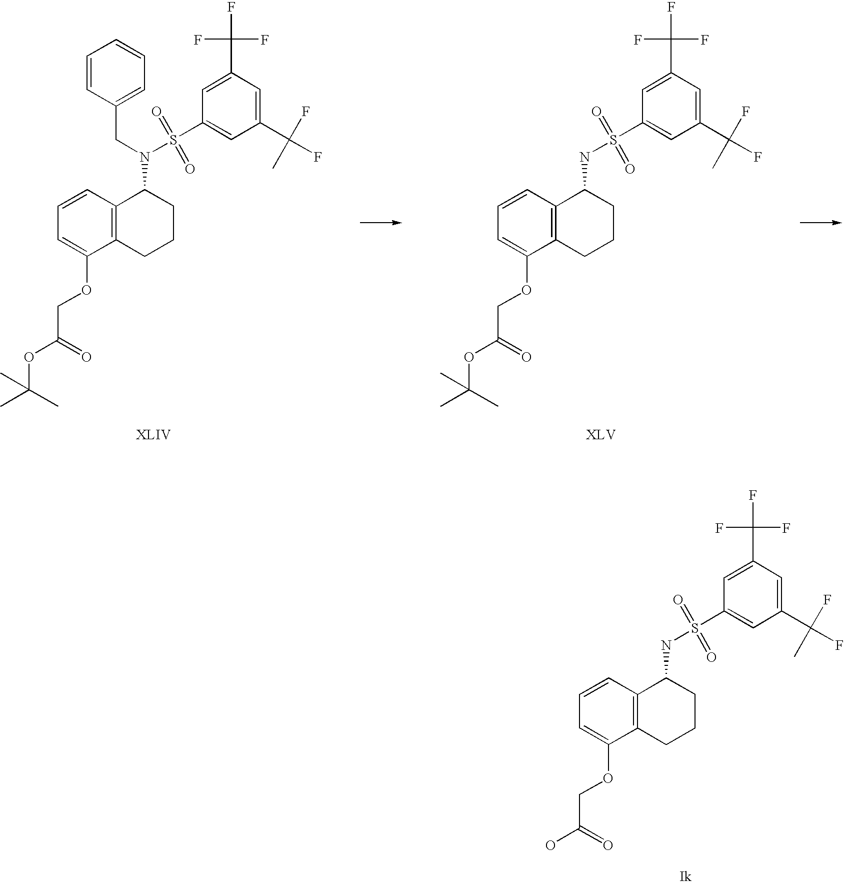

Compound of interest Ik can be prepared according to Scheme 10. Benzylation of the sulfonamide XXII with bromomethyl-benzene (XLI) gives the derivative XLII. A Stille coupling reaction between the aryl bromide XLII and 1-ethoxy-vinyltributyltin (XXIV), followed by acidic workup, produces the ketone XLIII, which can then be transformed to the gem-difluoride XLIV upon treatment with nucleophilic fluorinating sources. Debenzylation of the gem-difluoro derivative XLIV gives the N—H derivative XLV. Ester hydrolysis of the N—H derivative XLV generates the compound of interest Ik.

Benzylation of the sulfonamide XXII to produce the corresponding derivative XLII can be achieved by treating XXII with bromomethyl-benzene (XLI) in the presence of a weak base such as potassium carbonate or sodium carbonate, in an inert solvent such as N,N-dimethylformamide, acetonitrile, or tetrahydrofuran, at a temperature around 70° C. for several hours.

The ketone XLIII can be obtained by the Stille coupling reaction between the bromo derivative XLII and 1-ethoxy-vinyltributyltin (XXIV), followed by acidic hydrolysis with hydrochloric acid at room temperature to 70° C. for a period of 30 minutes to 18 hours in water or a mixture of water and tetrahydrofuran. The Stille coupling reaction is typically carried out in the presence of a palladium catalyst such as tetrakis(triphenylphosphine)palladium(0) (Pd(PPh₃)₄) or [1,1′-bis(diphenylphosphino)ferrocene]dichloropalladium(II) (PdCl₂(dppf)), in an inert solvent such as N,N-dimethylformamide, toluene, dioxane, acetonitrile, or mixtures thereof, at a temperature between 80 and 150° C. for 1 to 18 hours under an argon atmosphere. Alternatively, the reaction can be carried out in the presence of tris(dibenzylideneacetone)dipalladium(0) (Pd₂(dba)₃), and triphenylarsine (Ph₃As).

Transformation of the ketone XLIII to the gem-difluoride derivative XLIV can be accomplished with nucleophilic fluorinating sources such as diethylaminosulfur trifluoride (DAST), bis(2-methoxyethyl)aminosulfur trifluoride, (CH₃OCH₂CH₂)₂NSF₃ (Deoxo-Fluor reagent), α,α-difluoroamines, or N,N-diethyl-α,α-difluoro-(m-methylbenzyl)amine (DFMBA), either with or without a suitable solvent such as tetrahydrofuran, dichloromethane, or mixtures thereof, at a temperature between room temperature and 180° C. for several hours (reference: Lal, G. S. et al., J. Org. Chem. 64 (1999) 7048).

Debenzylation of the derivative XLIV to generate the N—H sulfonamide XLV can be achieved by treating the XLIV with formic acid ammonium salt in the presence of Palladium on carbon in a suitable organic solvent such as ethanol at a temperature around 60° C. for several hours.

Ester hydrolysis of XLV produces the compound of interest Ik. The reaction can be carried out in the presence of an aqueous inorganic base such as lithium hydroxide, sodium hydroxide or potassium hydroxide, in an inert solvent such as 1,4-dioxane or tetrahydrofuran, at room temperature for several hours, or at 40° C. for 1 hour.

### Examples

Although certain exemplary embodiments are depicted and described herein, the compounds of the present invention can be prepared using appropriate starting materials according to the methods described generally herein and/or by methods available to one of ordinary skill in the art.

### Materials and Instrumentation in General

Intermediates and final compounds were purified by either flash chromatography and/or preparative HPLC (high performance liquid chromatography). Flash chromatography was performed using (1) the Biotage SP1™ system and the Quad 12/25 Cartridge module from Biotage AB) or (2) the ISCO CombiFlash® chromatography instrument (from Teledyne Isco, Inc.); unless otherwise noted. The silica gel brand and pore size utilized were: (1) KP-SIL™ 60 Å, particle size: 40-60 micron (from Biotage AB); (2) Silica Gel CAS registry No: 63231-67-4, particle size: 47-60 micron; or (3) ZCX from Qingdao Haiyang Chemical Co., Ltd, pore size: 200-300 mesh or 300-400 mesh. Preparative HPLC was performed on a reversed phase column using an Xbridge™ Prep C₁₈ (5 μm, OBD™ 30×100 mm) column (from Waters Corporation), a SunFire™ Prep C₁₈ (5 μm, OBD™ 30×100 mm) column (from Waters Corporation), or a Varian Pursuit® C-18 column 20×150 mm (from Varian, Inc.).

Mass spectrometry (MS) or high resolution mass spectrometry (HRMS) was performed using a Waters® ZQ™ 4000 (from Waters Corporation), a Waters® Alliance® 2795-ZQ™ 2000 (from Waters Corporation), a Waters® Quattro micro™ API (from Waters Corporation), or an MDS ScieX™ API-2000™ n API (from MDS Inc.). Mass spectra data generally only indicates the parent ions unless otherwise stated. MS or HRMS data is provided for a particular intermediate or compound where indicated.

Nuclear magnetic resonance spectroscopy (NMR) was performed using a Varian® Mercury300 NMR spectrometer (for the HNMR spectrum acquired at 300 MHz) and a Varian® Inova400 NMR spectrometer (for the HNMR spectrum acquired at 400 MHz) both from Varian Inc. NMR data is provided for a particular intermediate or compound where indicated.

The microwave assisted reactions were carried out in a Biotage Initiator™ Sixty (or its early models) (from Biotage AB) or by a CEM Discover® model (with gas addition accessory) (from CEM Corporation).

Chiral separation was performed by supercritical fluid chromatography (SFC) using a Multigram® III instrument (from Thar Technologies, Inc.).

All reactions involving air-sensitive reagents were performed under an inert atmosphere. Reagents were used as received from commercial suppliers unless otherwise noted.

### Part I: Preparation of Preferred Intermediates

### Preparation of 3-fluoro-5-trifluoromethyl-benzenesulfonyl chloride

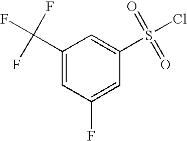

A mixture of 3-fluoro-5-trifluoromethyl-phenylamine (9.7 g, 54 mmol) in trifluoroacetic acid (100 mL) was cooled at 0° C. To the mixture was slowly added concentrated hydrochloric acid (10 mL), followed by a solution of sodium nitrite (4.7 g, 68 mmol) in water (5 mL) dropwise over 20 minutes. The mixture was stirred for another 10 minutes at 0° C., and then poured into a stirred mixture of acetic acid (120 mL), sulfurous acid (0.94 N aqueous sulfur dioxide solution, 120 mL), copper(II) chloride (9.2 g, 93 mmol) and copper(I) chloride (100 mg, 0.74 mmol) at 0° C. The resulting reaction mixture was allowed to warm to room temperature and stirred for 15 hours. Water (200 mL) was added, and the resulting mixture was extracted with ethyl acetate (100 mL×3). The combined organic layers were dried over sodium sulfate, filtered through a glass funnel and concentrated in vacuo. The residue was purified by column chromatography (20% ethyl acetate in petroleum ether) to afford 3-fluoro-5-trifluoromethyl-benzenesulfonyl chloride (3.7 g, 26%) as a white solid (reference: Cherney, R. J. et al., J. Med. Chem. 46 (2003) 1811). ¹H NMR (400 MHz, CDCl₃) δ ppm 8.15 (s, 1H); 7.97-7.99 (d, J=4.0 Hz, 1H); 7.74-7.76 (d, J=4.0 Hz, 1H).

### Preparation of 3,5-di-tert-butyl-benzenesulfonyl chloride

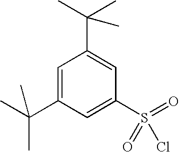

To 1,3,5-tri-tert-butyl-benzene (1.5 g, 6.1 mmol) was added chlorosulfonic acid (4 mL) at 0° C. After being stirred at 0° C. for 30 minutes, the reaction mixture was warmed to room temperature and stirred for 1 hour. Then mixture was then poured into ice water (50 mL) and extracted with dichloromethane (20 mL×3). The combined organic layers were dried over sodium sulfate and concentrated in vacuo. The residue was purified by column chromatography (0-20% ethyl acetate in petroleum ether) to afford 3,5-di-tert-butyl-benzenesulfonyl chloride (880 mg, 50%) as a yellow solid [reference: Guthrie, R. D. et al. Aust. J. Chem. 40 (1987) 2133; Ris, Cornell is et al. J. Chem. Soc. Perkin Trans II (1975) 1438].

### Preparation of 3-methoxy-5-trifluoromethyl-benzenesulfonyl chloride

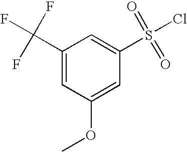

3-methoxy-5-trifluoromethyl-phenylamine (10 g, 54 mmol) was added to trifluoroacetic acid (100 mL) in a 250 mL flask, and the mixture was cooled to 0° C. Concentrated hydrochloric acid (10 mL) was then added slowly to the reaction mixture, followed by the dropwise addition of a solution of sodium nitrite (4.7 g, 68 mmol) in water (5 mL) over 20 min. The mixture was stirred for another 10 minutes at 0° C., and then poured into a stirred mixture of acetic acid (120 mL), sulfurous acid (0.94 N aqueous sulfur dioxide solution, 120 mL, 113 mmol), copper(II) chloride (9.2 g, 68 mmol) and copper(I) chloride (100 mg, 1 mmol) at 0° C. The reaction mixture was allowed to warm to room temperature and stirred for 15 hours, and then treated with water (200 mL). The aqueous layer was extracted with ethyl acetate (100 mL×3). The combined organic layers were dried over sodium sulfate, filtered through a glass funnel and concentrated in vacuo. The residue was purified by column chromatography (20% ethyl acetate in petroleum ether) to afford 3-methoxy-5-trifluoromethyl-benzenesulfonyl chloride (3.9 g, 27%) as a white solid [reference: Cherney, R. J. et al., J. Med. Chem. 46 (2003) 1811]. ¹H NMR (400 MHz, CDCl₃) δ ppm 7.89 (s, 1H); 7.70 (s, 1H); 7.50 (s, 1H); 4.00 (s, 3H).

### Preparation of N-methyl-3,5-bis-trifluoromethyl-benzenesulfonamide (Typical Preparation for Non-Commercial N-methylsulfonamides XII)

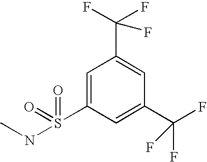

A solution of 5.0 g (15.69 mmol) of 3,5-bis-trifluoromethyl-benzenesulfonyl chloride in 25 mL of THF was added dropwise to a cold (0-5° C.) 40% aqueous solution of methylamine (3.0 g, 38.63 mmol) over 20 minutes. The resulting reaction mixture was stirred for an additional 1 hour at 0-5° C., and then quenched with water (20 mL), and extracted with methyl tert-butyl ether (25 mL). The organic layer was separated and washed with 2×20 mL of water, then concentrated to a volume of approximately 20 mL. Heptane (50 mL) was added, and the resulting mixture was concentrated at 40° C./90 torr to remove methyl tert-butyl ether to a total volume of 60 mL. Heptane addition and concentration was repeated a second time. The resulting precipitate was filtered and washed with heptane, then dried under vacuum overnight, to furnish 4.42 g of a white solid, which was used without further purification.

### Part II: Preparation of Compounds of Interest

### Example 1-1

### Preparation According to Scheme 1

### {(R)-5-[(3-fluoro-5-trifluoromethyl-benzenesulfonyl)-methyl-amino]-5,6,7,8-tetrahydro-naphthalen-1-yloxy}-acetic acid

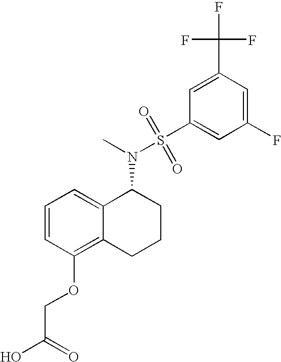

### 5-hydroxy-3,4-dihydro-2H-naphthalen-1-one (III)

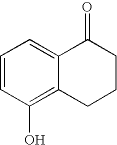

To a mixture of 1,5-dihydroxynaphthalene (25.0 g, 156 mmol) in isopropanol (150 mL) and an aqueous (40 mL) solution of sodium hydroxide (6.3 g, 157 mmol) was added 10% palladium on carbon (3.9 g) at room temperature. The reaction mixture was treated under 100 psi hydrogen in a Parr autoclave (from Parr Instrument Company) at 80° C. for 20 hours. After being cooled to room temperature, the reaction mixture was filtered through a pad of Celite® (a diatomite filter from World Minerals Inc.), and then washed with isopropanol (200 mL). The combined filtrates were treated with charcoal at 50° C. for 1 hour, and then were filtered through a pad of Celite® (diatomite filter). Isopropanol was removed, and the resulting solution was adjusted to a pH of about 2 by the slow addition of concentrated hydrochloric acid, during which a solid precipitate appeared. The solid was collected, and washed with water (100 mL×2), dried over high vacuum at 50° C. to give 5-hydroxy-3,4-dihydro-2H-naphthalen-1-one (15.0 g, 60%) as dark brown solid, which was used in the next step without further purification. MS cald. (calculated) for C₁₀H₁₀O₂ 162, obsd. (observed) 163 [(M+H)⁺].

### (5-Oxo-5,6,7,8-tetrahydro-naphthalen-1-yloxy)-acetic acid tert-butyl ester (V)

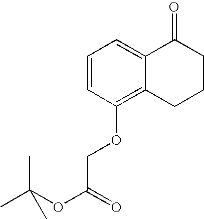

To a stirred mixture of 5-hydroxy-3,4-dihydro-2H-naphthalen-1-one (10.0 g, 61.7 mmol) and cesium carbonate (58.5 g, 180 mmol) in acetonitrile (300 mL) was added tert-butyl bromoacetate (29.0 g, 148 mmol) at room temperature under nitrogen. After overnight stirring at room temperature, the reaction mixture was filtered through a pad of Celite® (a diatomite filter), and washed with ethyl acetate (100 mL). The combined filtrates were concentrated under reduced pressure. The residue was partitioned between ethyl acetate (500 mL) and water (200 mL×3). The organic layer was concentrated under reduced pressure. Column chromatography (silica gel, 100-200 mesh, 5-10% ethyl acetate in hexane) gave (5-oxo-5,6,7,8-tetrahydro-naphthalen-1-yloxy)-acetic acid tert-butyl ester (12.1 g, 71%). MS cald. for C₁₆H₂₀O₄ 276, obsd. 277 [(M+H)⁺].

### ((R)-5-amino-5,6,7,8-tetrahydro-naphthalen-1-yloxy)-acetic acid tert-butyl ester hydrochloride salt (VI)

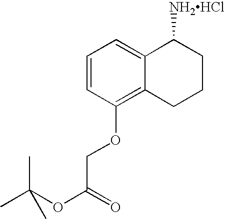

To a stirred solution of (5-oxo-5,6,7,8-tetrahydro-naphthalen-1-yloxy)-acetic acid tert-butyl ester (76.6 g, 0.28 mol) in methanol (1100 mL) was added ammonium acetate (299.0 g, 3.88 mol), followed by a dropwise addition of a solution of sodium cyanoborohydride (17.4 g, 0.28 mol) in methanol (100 mL) at room temperature under nitrogen. The reaction mixture was stirred at room temperature for 4 days until no remaining traces of starting material was detected (as monitored by TLC, ethyl acetate:methanol=10:1). The reaction mixture was then concentrated under reduced pressure. To the residue was added saturated sodium carbonate solution (700 mL), and the resulting solution was extracted with dichloromethane (1000 mL×3). The combined organic layers were dried over anhydrous sodium sulfate, filtered, and concentrated in vacuo to afford a semi-solid crude product, which was triturated with diethyl ether (150 mL), and then treated with 8M hydrochloric acid in ethyl acetate (70 mL). The resulting white precipitate was filtered, and washed with anhydrous diethyl ether, then dried at 55° C. in an oven to afford (5-amino-5,6,7,8-tetrahydro-naphthalen-1-yloxy)-acetic acid tert-butyl ester hydrochloride salt (54 g, 62%) as a white solid. Chiral separation by supercritical fluid chromatography (SFC) (using Thar Technologies, Inc.'s Multigram® III instrument, Daicel® OD column 5×25 cm, 30% methanol, 200 mL/min) afforded the R-(5-amino-5,6,7,8-tetrahydro-naphthalen-1-yloxy)-acetic acid tert-butyl ester hydrochloride salt. MS cald. for C₁₆H₂₃NO₃ 277, obsd. 278 (ESI⁺) [(M+H)⁺].

The assignment of absolute stereochemistry was established by x-ray structure determination of the corresponding 4-iodophenylsulfonamide derivative.

### [(R)-5-(3-fluoro-5-trifluoromethyl-benzenesulfonylamino)-5,6,7,8-tetrahydro-naphthalen-1-yloxy]-acetic acid tert-butyl ester

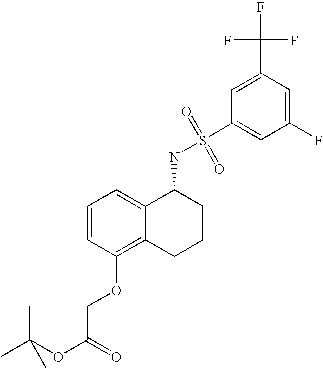

To a solution of ((R)-5-amino-5,6,7,8-tetrahydro-naphthalen-1-yloxy)-acetic acid tert-butyl ester hydrochloride salt (1.04 g, 3.30 mmol) and N,N-diisopropylethylamine (1.36 mL, 7.86 mmol) in dry tetrahydrofuran (15 mL) was added 3-fluoro-5-(trifluoromethyl)-benzenesulfonyl chloride (0.867 g, 3.30 mmol) at room temperature. The reaction mixture was stirred at room temperature overnight, and then concentrated. The remaining residue was partitioned between water and ethyl acetate. The collected organic layers were washed with water, dried over magnesium sulfate, filtered, and evaporated in vacuo. Flash chromatography (RediSep® Flash column from Teledyne Isco, Inc., 230-400 mesh, 0-10% ethyl acetate in hexane) gave [(R)-5-(3-fluoro-5-trifluoromethyl-benzenesulfonylamino)-5,6,7,8-tetrahydro-naphthalen-1-yloxy]-acetic acid tert-butyl ester (867 mg, 52%). MS cald. for C₂₄H₂₅F₄NO₅S 503, obsd. 504 (ESI⁺) [(M+H)⁺].

### {(R)-5-[(3-fluoro-5-trifluoromethyl-benzenesulfonyl)-methyl-amino]-5,6,7,8-tetrahydro-naphthalen-1-yloxy}-acetic acid tert-butyl ester

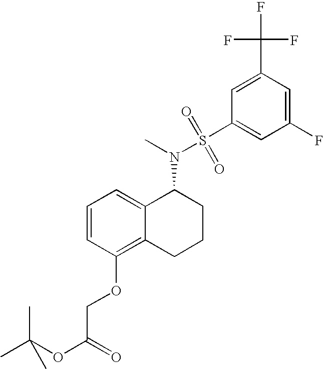

To a solution of [(R)-5-(3-fluoro-5-trifluoromethyl-benzenesulfonylamino)-5,6,7,8-tetrahydro-naphthalen-1-yloxy]-acetic acid tert-butyl ester (800 mg, 1.59 mmol) in N,N-dimethylformamide (5 mL) were added potassium carbonate (483 mg, 3.5 mmol) and iodomethane (200 μL, 3.18 mmol) at room temperature, and the resulting mixture was stirred overnight. The reaction mixture was then partitioned between ethyl acetate and water. The collected organic layers were washed with water (4×), then brine (2×), dried over magnesium sulfate, filtered, and concentrated in vacuo. Flash chromatography (RediSep® Flash column from Teledyne Isco, Inc., 230-400 mesh, 0-40% ethyl acetate in hexane) gave {(R)-5-[(3-fluoro-5-trifluoromethyl-benzenesulfonyl)-methyl-amino]-5,6,7,8-tetrahydro-naphthalen-1-yloxy}-acetic acid tert-butyl ester (577 mg, 70%). MS cald. for C₂₄H₂₇F₄NO₅S 517, obsd. 518 (ESI⁺) [(M+H)⁺].

### {(R)-5-[(3-fluoro-5-trifluoromethyl-benzenesulfonyl)-methyl-amino]-5,6,7,8-tetrahydro-naphthalen-1-yloxy}-acetic acid

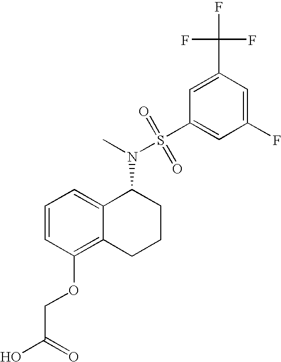

To a solution of {(R)-5-[(3-fluoro-5-trifluoromethyl-benzenesulfonyl)-methyl-amino]-5,6,7,8-tetrahydro-naphthalen-1-yloxy}-acetic acid tert-butyl ester (50 mg, 0.097 mmol) in tetrahydrofuran (0.5 mL) was added 2 N sodium hydroxide solution (1 mL, 2 mmol). The reaction mixture was stirred at room temperature overnight. Tetrahydrofuran was removed under reduced pressure. The remaining solution was diluted with water, and washed with ether. The collected aqueous layer was acidified with dilute hydrochloric acid to a pH of about 2, and then extracted three times with ethyl acetate. The organic layers were dried over sodium sulfate, filtered, and concentrated under reduced pressure to give pure {(R)-5-[(3-fluoro-5-trifluoromethyl-benzenesulfonyl)-methyl-amino]-5,6,7,8-tetrahydro-naphthalen-1-yloxy}-acetic acid (13 mg, 29%). HRMS cald. for C₂₀H₁₉F₄NO₅S (ESI⁺)[(M+Na)⁺] 484.0812, obsd. 484.0811; ¹H NMR (300 MHz, DMSO-d₆) δ ppm 13.02 (br. s, 1H), 8.18 (t, J=9.2 Hz, 2H), 8.04 (s, 1H), 7.11 (t, J=8.1 Hz, 1H), 6.71 (d, J=8.1 Hz, 1H), 6.67 (d, J=8.1 Hz, 1H), 5.14-5.26 (m, 1H), 4.66 (s, 2H), 2.73 (d, J=16.9 Hz, 1H), 2.56 (s, 3H), 2.28-2.46 (m, 1H), 1.83 (br. s, 1H), 1.54-1.77 (m, 2H), 1.40-1.54 (m, 1H).

### Alternative Preparation of ((R)-5-amino-5,6,7,8-tetrahydro-naphthalen-1-yloxy)-acetic acid tert-butyl ester hydrochloride salt (VI) According to Scheme 2

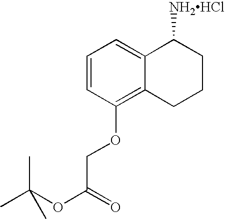

### ((S)-5-hydroxy-5,6,7,8-tetrahydro-naphthalen-1-yloxy)-acetic acid tert-butyl ester (XI)

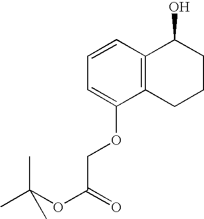

To a flask containing 124 mg (0.203 mmol) of di-mu-chlorobis[(p-cymene)chlororuthenium(II) ([RuCl₂(C₁₀H₁₄)]₂, Strem Chemicals, Inc., CAS No. 52462-29-0) and 153 mg (0.416 mmol) of (1S,2S)-(+)-N-p-tosyl-1,2-diphenylethylenediamine (Aldrich, CAS No. 167316-27-0) was added 50 mL of a pre-formed mixture of formic acid and triethylamine (in 5:2 molar ratio), and the resulting mixture was stirred at room temperature for 45 minutes (gas evolution was observed). Then 10 g (36.19 mmol) of (5-oxo-5,6,7,8-tetrahydro-naphthalen-1-yloxy)-acetic acid tert-butyl ester (V, prepared as described above) was added, and the reaction mixture was stirred at 42° C. internal temperature. Upon gas evolution and foaming, the reaction mixture was cooled to 33° C. internal temperature over 1 hour, and then stirred for an additional 24 hours at 33° C. The reaction mixture was then cooled in an ice-water bath, diluted with 50 mL of de-ionized water, and extracted with 100 mL of toluene. The organic layer was separated and washed with 1 M aqueous citric acid (50 mL), saturated aqueous sodium bicarbonate (50 mL), and water (50 mL). The organic phase was then dried over MgSO₄, and concentrated azeotropically at 35° C./20 mmHg to a total volume of 30 mL. The resulting solution was co-evaporated with 2×100 mL of toluene to a total volume of 20 mL (product and toluene), which was used in the next step without further purification.

### ((R)-5-azido-5,6,7,8-tetrahydro-naphthalen-1-yloxy)-acetic acid tert-butyl ester

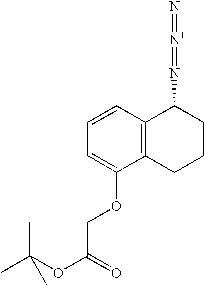

The toluene solution of chiral alcohol XI prepared above (36.19 mmol, assumed 100% conversion) was diluted with an additional 100 mL of toluene, and cooled in an ice-water bath, then treated with diphenylphosphoryl azide (13.64 g, 49.57 mmol). To this solution was added 1,8-diazabicyclo[5.4.0]undec-7-ene (DBU, 8.0 g, 52.46 mmol), dropwise over 20 minutes at such a rate so as to maintain the internal temperature between 1-4° C. The reaction mixture was then stirred at an internal temperature of 1-2° C. for an additional 45 minutes, then warmed to room temperature (with a water bath), and stirred at room temperature overnight. After 20 hours, the reaction mixture was treated with ice-cold water (50 mL), while maintaining the internal temperature below 24° C. The organic layer was separated and washed with 1 M aqueous citric acid solution (50 mL), saturated aqueous sodium bicarbonate (50 mL), and water (50 mL). The resulting organic phase was then concentrated under vacuum at 20 mmHg/26° C., to provide 15 g of an oil, which was used in the next step without further purification.

### ((R)-5-amino-5,6,7,8-tetrahydro-naphthalen-1-yloxy)-acetic acid tert-butyl ester hydrochloride salt (VI)

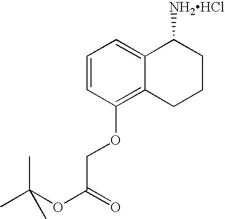

To a solution of ((R)-5-azido-5,6,7,8-tetrahydro-naphthalen-1-yloxy)-acetic acid tert-butyl ester prepared above (36.19 mmol, assumed 100% conversion) in 100 mL of methanol in a 300 mL Parr-reactor was added water (1.6 mL) and 5% Pd/C (1.4 g). The reaction mixture was stirred under a 350 psi pressure of hydrogen. After 90 minutes, the reaction was filtered through a pad of Celite, washed with methanol, and concentrated in vacuo to provide 16.0 g of an oil. The crude oil was dissolved in 10 mL of methanol and 50 mL of methyl tert-butyl ether. Water was removed azeotropically, to provide 14.0 g of an oil, which was dissolved in 10 m L of methanol, and 50 m L of methyl tert-butyl ether. To th is solution was added a solution of chlorotrimethylsilane (5.722 mL, 43.42 mmol) in 50 mL of methyl tert-butyl ether at room temperature, dropwise over 40 minutes. The resulting mixture was stirred for 2 hours. The resulting precipitate was filtered, to provide 8.8 g (78% yield over 3 steps) of ((R)-5-amino-5,6,7,8-tetrahydro-naphthalen-1-yloxy)-acetic acid tert-butyl ester hydrochloride salt (VI).

### Examples 1-2 to 1-9

The following examples 1-2 to 1-9 were prepared in an analogous manner to example 1-1 starting with naphthalene-1,5-diol and the appropriate commercially available or prepared aryl sulfonyl chlorides.

| | | | MS | |
|---|---|---|---|---|
| Example | Systematic | ¹H NMR (300 MHz, | (ESI+, | |
| No. | Name | CDCl₃) δ ppm | M + Na⁺) | Structure |
| 1-2 | {(R)-5-[(3,5-di- tert-butyl- benzenesulfonyl)- methyl- amino]- 5,6,7,8- tetrahydro- naphthalen-1- yloxy}-acetic acid | 7.73 (s, 2 H), 7.64 (s, 1 H), 7.06 (t, J = 7.8 Hz, 1 H), 6.89 (d, J = 7.8 Hz, 1 H), 6.59 (d, J = 7.8 Hz, 1 H), 5.12- 5.25 (m, 1 H), 4.66 (s, 2 H), 2.76-2.95 (m, 1 H), 2.56 (s, 3 H), 2.42-2.66 (m, 1 H), 1.85-2.01 (m, 1 H), 1.53-1.77 (m, 3 H), 1.37 (s, 18 H) | 510.2281 | 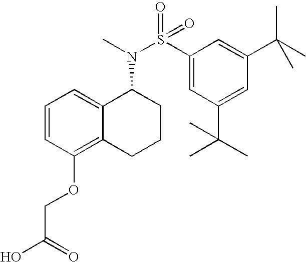 |
| 1-3 | {(R)-5-[(3,5- bis- methanesulfonyl- benzenesulfonyl)- methyl- amino]- 5,6,7,8- tetrahydro- naphthalen-1- yloxy}-acetic acid | (DMSO-d₆) 13.00 (br. s, 1 H), 8.69 (s, 2 H), 8.67 (br. s, 1 H), 7.13 (t, J = 8.0 Hz, 1 H), 6.73 (d, J = 8.0 Hz, 1 H), 6.72 (d, J = 8.0 Hz, 1 H), 5.23-5.34 (m, 1 H), 4.67 (s, 2 H), 3.49 (s, 6 H), 2.65- 2.82 (m, 1 H), 2.57 (s, 3 H), 2.33-2.48 (m, 1 H), 1.43-1.94 (m, 4 H) | 554.0586 | 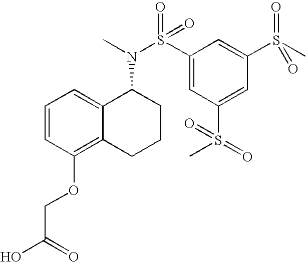 |
| 1-4 | {(R)-5-[(3- methoxy-5- trifluoromethyl- benzenesulfonyl)- methyl- amino]- 5,6,7,8- tetrahydro- naphthalen-1- yloxy}-acetic acid | 7.73 (s, 1 H), 7.58 (s, 1 H), 7.32-7.38 (m, 1 H), 7.12 (t, J = 7.8 Hz, 1 H), 6.92 (d, J = 7.8 Hz, 1 H), 6.62 (d, J = 7.8 Hz, 1 H), 5.19- 5.28 (m, 1 H), 4.69 (s, 2 H), 3.93 (s, 3 H), 2.78-3.01 (m, 1 H), 2.62 (s, 3 H), 2.44- 2.58 (m, 1 H), 1.96 (br. s, 1 H), 1.70 (br. s, 3 H) | 496.1014 | 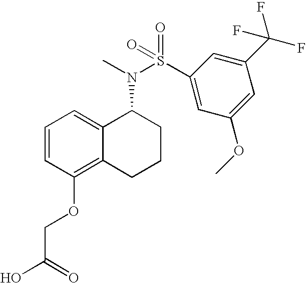 |
| 1-5 | {(R)-5-[(3- bromo-5- trifluoromethyl- benzenesulfonyl)- methyl- amino]- 5,6,7,8- tetrahydro- naphthalen-1- yloxy}-acetic acid | (DMSO-d₆) 12.99 (s, 1 H), 8.41 (s, 2 H), 8.19 (s, 1 H), 7.10 (t, J = 8.0 Hz, 1 H), 6.71 (d, J = 8.0 Hz, 1 H), 6.66 (d, J = 8.0 Hz, 1 H), 5.11-5.30 (m, 1 H), 4.67 (s, 2 H), 2.73 (d, J = 17.2 Hz, 1 H), 2.55 (s, 3 H), 2.31- 2.47 (m, 1 H), 1.83 (br. s, 1 H), 1.40- 1.78 (m, 3 H) | 544.0010 | 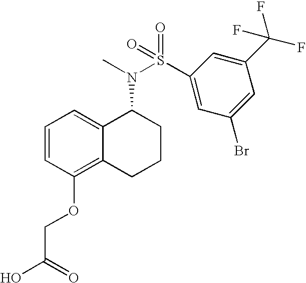 |
| 1-6 | {(R)-5-[(3,5- bis- trifluoromethyl- benzenesulfonyl)- methyl- amino]- 5,6,7,8- tetrahydro- naphthalen-1- yloxy}-acetic acid | 8.35 (s, 2 H), 8.11 (s, 1 H), 7.13 (t, J = 8.0 Hz, 1 H), 6.87 (d, J = 7.8 Hz, 1 H), 6.63 (d, J = 8.2 Hz, 1 H), 5.20- 5.34 (m, 1 H), 4.70 (s, 2 H), 2.79-3.00 (m, 1 H), 2.63 (s, 3 H), 2.40-2.59 (m, 1 H), 1.86-2.10 (m, 1 H), 1.55-1.85 (m, 3 H) | 624* | 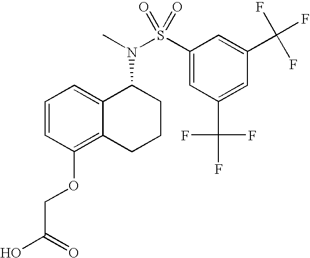 |
| 1-7^{a} | {(R)-5-[(3,-5- dichloro- benzenesulfonyl)- methyl- amino]- 5,6,7,8- tetrahydro- naphthalen-1- yloxy}-acetic acid | (DMSO-d₆, 400 MHz): (br. s, 1 H), 8.04 (t, J = 1.7 Hz, 1 H), 7.95 (d, J = 1.7 Hz, 2 H), 7.11 (t, J = 8.0 Hz, 1 H), 6.71 (d, J = 8.0 Hz, 1 H), 6.67 (d, J = 8.0 Hz, 1 H), 5.11-5.24 (m, 1 H), 4.66 (s, 2 H), 2.71 (br. s, 1 H), 2.55 (s, 3 H), 2.36-2.47 (m, 1 H), 1.78-1.93 (m, 1 H), 1.44-1.78 (m, 3 H) | 442/444^{#} | 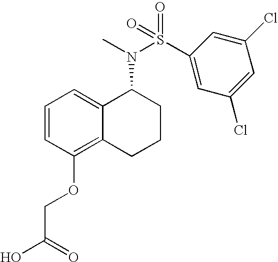 |
| 1-8^{a} | {(R)-5-[(3,-5- difluoro- benzenesulfonyl)- methyl- amino]- 5,6,7,8- tetrahydro- naphthalen-1- yloxy}-acetic acid | (DMSO-d₆, 400 MHz): 13.01 (br. s, 1 H), 7.66-7.75 (m, 3 H), 7.11 (t, J = 8.0 Hz, 1 H), 6.71 (d, J = 8.0 Hz, 1 H), 6.67 (d, J = 8.0 Hz, 1 H), 5.08- 5.18 (m, 1 H), 4.66 (s, 2 H), 2.66-2.79 (m, 1 H), 2.55 (s, 3 H), 2.36-2.47 (m, 1 H), 1.84 (br. s, 1 H), 1.40-1.78 (m, 3H) | 410^{#} | 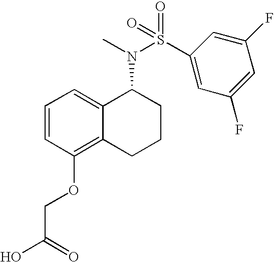 |
| 1-9^{a} | {(R)-5-[(3,5- dimethyl- benzenesulfonyl)- methyl- amino]- 5,6,7,8- tetrahydro- naphthalen-1- yloxy}-acetic acid | (DMSO-d₆, 400 MHz): 12.99 (br. s, 1 H), 7.50 (s, 2 H), 7.34 (s, 1 H), 7.09 (t, J = 8.0 Hz, 1 H), 6.72 (d, J = 8.0 Hz, 1 H), 6.69 (d, J = 8.0 Hz, 1 H), 4.98-5.10 (m, 1 H), 4.65 (s, 2 H), 2.70 (br. s, 1 H), 2.48 (s, 3 H), 2.39-2.46 (m, 1 H), 2.38 (s, 6 H), 1.83 (br. s, 1 H), 1.42- 1.70 (m, 3 H) | 402^{#} | 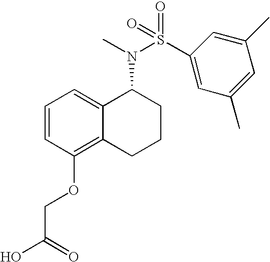 |

^{a}Prepared as a racemate, then resolved using chiral chromatography
*(ESI⁻) [(M + TFA − H)⁻]
^{#}(ESI⁻) (M − H)⁻

### Alternative Preparation of {(R)-5-[(3,5-bis-trifluoromethyl-benzenesulfonyl)-methyl-amino]-5,6,7,8-tetrahydro-naphthalen-1-yloxy}-acetic acid (Example 1-6) Using the Mitsunobu Reaction, Followed by Hydrolysis, According to Scheme 2:

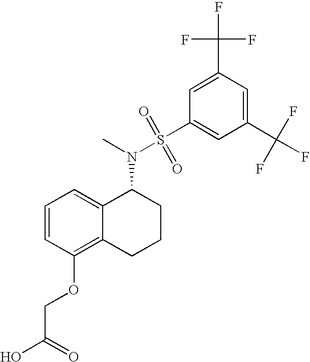

A solution of ((S)-5-hydroxy-5,6,7,8-tetrahydro-naphthalen-1-yloxy)-acetic acid tert-butyl ester (XI, 9.06 g, 32.55 mmol), N-methyl-3,5-bis-trifluoromethyl-benzenesulfonamide (10.0 g, 32.55 mmol), and triphenylphosphine (10.25 g, 39.06 mmol) in 2-methyl-tetrahydro-furan (150 mL) was cooled to −20° C. To this solution was added diisopropyl azodicarboxylate (7.69 mL, 39.06 mmol) dropwise over 15 minutes, so as to maintain the internal reaction temperature at approximately −20° C. The reaction mixture was stirred at about −20° C. for 2 hours, then warmed to −10° C. over 1 hour to ensure complete consumption of the alcohol. The reaction mixture was then quenched with 110 mL of a methanol:water (4:3) solution, and extracted with 130 mL of heptane. The organic layer was separated and washed with 2×110 mL of methanol:water (4:3) solution (to remove triphenylphosphine oxide). The organic phase was then concentrated and the crude material was dissolved in 100 mL of THF. Lithium hydroxide (1 M solution, 162.8 mL, 162.8 mmol) was added, and the reaction mixture was heated at 50° C. for 7 hours. The reaction mixture was then cooled to room temperature, and stirred at room temperature overnight. HPLC analysis indicated complete hydrolysis. The resulting mixture was diluted with methyl tert-butyl ether (140 mL). The organic phase was separated and washed with 1 M lithium hydroxide (162.8 mL, 162.8 mmol), followed by 1 N hydrochloric acid (162.8 mL, 162.8 mmol), and water (200 mL). The organic layer was separated and dried over MgSO₄, filtered, and concentrated to a total volume of 50 mL. Methyl tert-butyl ether was added, and the resulting solution was concentrated to a total volume of 60 mL, then heated at reflux while heptane was added rapidly dropwise until crystallization occurred. The mixture was heated at reflux for 1 h, then cooled to room temperature and stirred overnight. The resulting precipitate was filtered and washed with a 1:9 mixture of methyl tert-butyl ether:heptane (20 mL), then heptane (20 mL). The residue was then dried to produce 10.62 g of {(R)-5-[(3,5-bis-trifluoromethyl-benzenesulfonyl)-methyl-amino]-5,6,7,8-tetrahydro-naphthalen-1-yloxy}-acetic acid as a white solid.

### Example 2-1

### ((R)-5-{methyl-[3-(propane-2-sulfinyl)-5-trifluoromethyl-benzenesulfonyl]-amino}-5,6,7,8-tetrahydro-naphthalen-1-yloxy)-acetic acid

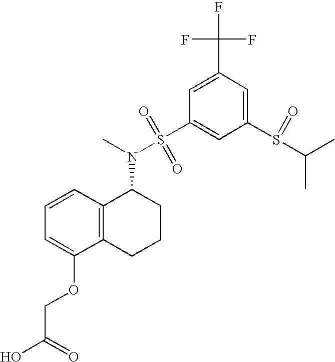

### {(R)-5-[(3-isopropylsulfanyl-5-trifluoromethyl-benzenesulfonyl)-methyl-amino]-5,6,7,8-tetrahydro-naphthalen-1-yloxy}-acetic acid tert-butyl ester

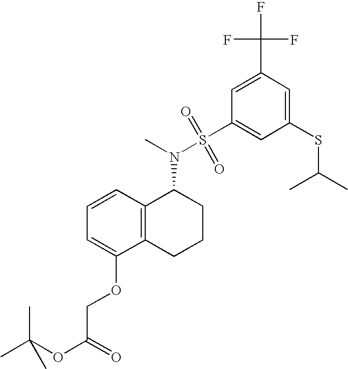

A mixture of {(R)-5-[(3-fluoro-5-trifluoromethyl-benzenesulfonyl)-methyl-amino]-5,6,7,8-tetrahydro-naphthalen-1-yloxy}-acetic acid tert-butyl ester (example 1-1, 5^{th} step) (150 mg, 0.29 mmol), potassium carbonate (300 mg, 2.17 mmol), and propane-2-thiol (165 mg, 2.17 mmol)) in N,N-dimethylformamide (2 mL) was heated at 150° C. for 30 minutes in a microwave oven. To the reaction mixture was added an aqueous solution of saturated ammonium chloride (10 mL) and the resulting solution was extracted with ethyl acetate (3×20 mL). The combined organic layers were washed with water (20 mL) and brine (20 mL), and then concentrated to afford {(R)-5-[( 3-isopropylsulfanyl-5-trifluoromethyl-benzenesulfonyl)-methyl-amino]-5,6,7,8-tetrahydro-naphthalen-1-yloxy}-acetic acid tert-butyl ester (160 mg, 96%) as a viscous oil, which was used in the next step without purification. MS cald. for C₂₇H₃₄F₃NO₅S₂ 573, obsd 574 (ESI⁺) [(M+H)⁺].

### ((R)-5-{methyl-[3-(propane-2-sulfinyl)-5-trifluoromethyl-benzenesulfonyl]-amino}-5,6,7,8-tetrahydro-naphthalen-1-yloxy)-acetic acid tert-butyl ester

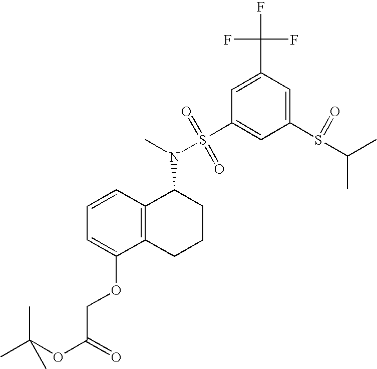

A solution of {(R)-5-[(3-isopropylsulfanyl-5-trifluoromethyl-benzenesulfonyl)-methyl-amino]-5,6,7,8-tetrahydro-naphthalen-1-yloxy}-acetic acid tert-butyl ester (160 mg, 0.28 mmol) and 3-chloroperoxybenzoic acid (85%, 200 mg, 0.99 mmol) in dichloromethane (30 mL) was stirred at room temperature for 4 hours. The reaction mixture was diluted with dichloromethane(150 mL) and then washed with an aqueous solution of sodium thiosulfate (50 mL) and saturated sodium carbonate (50 mL). The organic layers were concentrated in vacuo to afford {(R)-5-[(3-isopropylsulfinyl-5-trifluoromethyl-benzenesulfonyl)-methyl-amino]-5,6,7,8-tetrahydro-naphthalen-1-yloxy}-acetic acid tert-butyl ester (140 mg, 85%, contained a minor amount of the corresponding sulfonyl derivative) as a viscous oil, which was used in the next step without purification. MS cald. for C₂₇H₃₄F₃NO6S₂ 589, obsd 590 (ESI⁺) [(M+H)⁺].

### {(R)-5-[(3-isopropylsulfinyl-5-trifluoromethyl-benzenesulfonyl)-methyl-amino]-5,6,7,8-tetrahydro-naphthalen-1-yloxy}-acetic acid

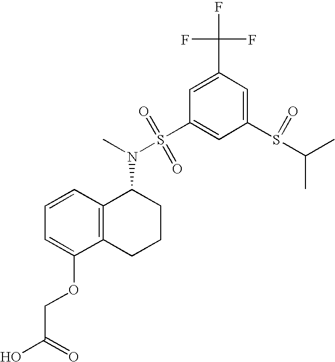

Starting with {(R)-5-[(3-isopropylsulfinyl-5-trifluoromethyl-benzenesulfonyl)-methyl-amino]-5,6,7,8-tetrahydro-naphthalen-1-yloxy}-acetic acid tert-butyl ester (80 mg, 0.14 mmol), and using the method analogous to the one described for example 1-1, {(R)-5-[(3-isopropylsulfinyl-5-trifluoromethyl-benzenesulfonyl)-methyl-amino]-5,6,7,8-tetrahydro-naphthalen-1-yloxy}-acetic acid was obtained as a crude mixture contaminated with a minor amount of the corresponding sulfonyl derivative. Preparative HPLC [SunFire™ Prep C₁₈ column from Waters Corporation (5 μM, OBD™ 30×100 mm, 0.5% TFA, 40-70% CH₃CN in water, 40 mL/min)], provided pure {(R)-5-[(3-isopropylsulfinyl-5-trifluoromethyl-benzenesulfonyl)-methyl-amino]-5,6,7,8-tetrahydro-naphthalen-1-yloxy}-acetic acid (15 mg, 20%) as a white solid. MS cald. for C₂₃H₂₆F₃NO₆S₂ 533, obsd. 534 (ESI⁺) [(M+H)⁺]; ¹H NMR (400 MHz, CD₃OD) δ ppm 8.44 (s, 1H), 8.34 (s, 1H), 8.27 (s, 1H), 7.07 (dt, J=8.08, 3.03 Hz, 1H), 6.70 (d, J=8.08 Hz, 1H), 6.72 (dd, J=12.88, 7.83 Hz, 1H), 5.24 (t, 1H), 4.66 (s, 2H), 3.11-3.20 (m, 1H), 2.85 (d, J=2.53 Hz, 1H), 2.63 (s, 3H), 2.47-2.58 (m, 1H), 1.89-1.99 (m, 1H), 1.58-1.77 (m, 3H), 1.38 (dd, J=7.07, 1.26 Hz, 3H), 1.04 (d, J=6.57 Hz, 3H).

### Example 3-1

### {(R)-5-[(3-cyclopentanesulfonyl-5-trifluoromethyl-benzenesulfonyl)-methyl-amino]-5,6,7,8-tetrahydro-naphthalen-1-yloxy}-acetic acid

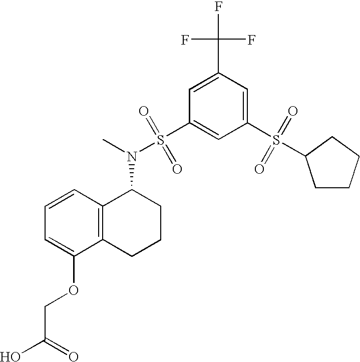

### {(R)-5-[(3-cyclopentylsulfanyl-5-trifluoromethyl-benzenesulfonyl)-methyl-amino]-5,6,7,8-tetrahydro-naphthalen-1-yloxy}-acetic acid tert-butyl ester

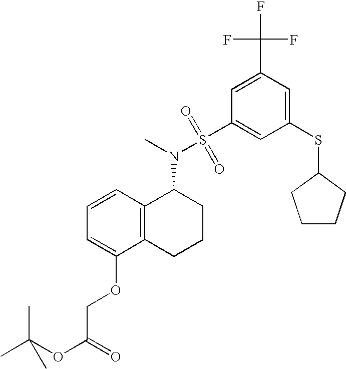

Starting with {(R)-5-[(3-fluoro-5-trifluoromethyl-benzenesulfonyl)-methyl-amino]-5,6,7,8-tetrahydro-naphthalen-1-yloxy}-acetic acid tert-butyl ester (example 1-1, 5^{th} step) and cyclopentanethiol, and using the method analogous to the one described for example 2-1, 1^{st} step, {(R)-5-[(3-cyclopentylsulfanyl-5-trifluoromethylbenzenesulfonyl)-methyl-amino]-5,6,7,8-tetrahydro-naphthalen-1-yloxy}-acetic acid tert-butyl ester (110 mg) was obtained as a viscous oil, which was used in the next step without purification. MS cald. for C₂₉H₃₆F₃NO₅S₂ 599, obsd. 600 (ESI⁺) [(M+H)⁺].

### {(R)-5-[(3-cyclopentanesulfonyl-5-trifluoromethyl-benzenesulfonyl)-methyl-amino]-5,6,7,8-tetrahydro-naphthalen-1-yloxy}-acetic acid tert-butyl ester

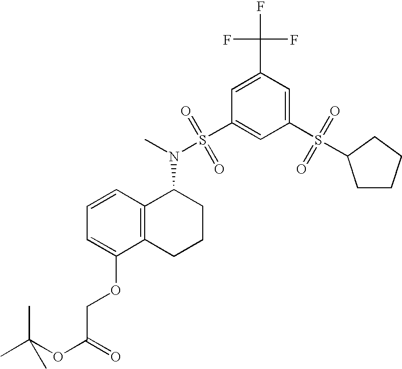

A mixture of {(R)-5-[(3-cyclopentylsulfanyl-5-trifluoromethyl-benzenesulfonyl)-methyl-amino]-5,6,7,8-tetrahydro-naphthalen-1-yloxy}-acetic acid tert-butyl ester (110 mg, 0.18 mmol) and m-chloroperoxybenzoic acid (85%, 300 mg, 1.48 mmol) in dichloromethane (20 mL) was stirred at room temperature for 4 hours. The reaction mixture was diluted with dichloromethane (100 mL) and then subsequently washed with an aqueous solution of sodium thiosulfate (50 mL) and saturated sodium carbonate (30 mL). The organic layer was concentrated to afford {(R)-5-[(3-cyclopentanesulfonyl-5-trifluoromethyl-benzenesulfonyl)-methyl-amino]-5,6,7,8-tetrahydro-naphthalen-1-yloxy}-acetic acid tert-butyl ester (100 mg, 88%) as a viscous oil, which was used in the next step without purification. MS cald. for C₂₉H₃₆F₃NO₇S₂ 631, obsd. 632 (ESI⁺) [(M+H)⁺].

### {(R)-5-[(3-cyclopentanesulfonyl-5-trifluoromethyl-benzenesulfonyl)-methyl-amino]-5,6,7,8-tetrahydro-naphthalen-1-yloxy}-acetic acid

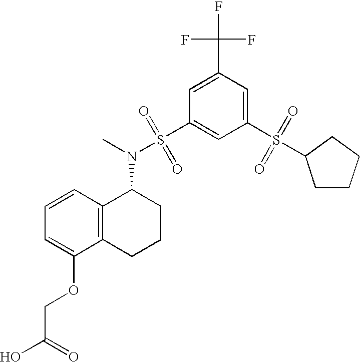

Starting with {(R)-5-[(3-cyclopentanesulfonyl-5-trifluoromethyl-benzene sulfonyl)-methyl-amino]-5,6,7,8-tetrahydro-naphthalen-1-yloxy}-acetic acid tert-butyl ester, and using the method analogous to the one described for example 1-1, {(R)-5-[(3-cyclopentanesulfonyl-5-trifluoromethyl-benzenesulfonyl)-methyl-amino]-5,6,7,8-tetrahydro-naphthalen-1-yloxy}-acetic acid (15 mg) was obtained as a white solid. MS cald. for C₂₅H₂₈F₃NO₇S₂ 575, obsd. 576 (ESI⁺) [(M+H)⁺]; ¹H NMR (400 MHz, CD₃OD) δ ppm 8.61 (s, 1H), 8.50 (d, J=7.83 Hz, 2H), 7.08 (t, J=7.96 Hz, 1H), 6.72 (dd, J=11.75, 7.96 Hz, 2H), 5.29 (t, 1H), 4.67 (s, 2H), 3.85-3.95 (m, 1H), 2.86 (d, 1H), 2.63 (s, 3H), 2.47-2.59 (m, 1H), 1.87-2.05 (m, 6H), 1.61-1.82 (m, 6H).

### Example 3-2 and 3-3

The following examples 3-2 and 3-3 were prepared in an analogous manner as described for example 3-1 using {(R)-5-[(3-fluoro-5-trifluoromethyl-benzene sulfonyl)-methyl-amino]-5,6,7,8-tetrahydro-naphthalen-1-yloxy}-acetic acid tert-butyl ester and the commercially available alkyl thiols.

| | | | MS | |
|---|---|---|---|---|
| Example | Systematic | ¹H NMR (400 MHz, | (ESI⁺, | |
| No. | Name | CD₃OD) δ ppm | [(M + H)⁺] | Structure |
| 3-2 | ((R)-5-{methyl- [3-(propane-2- sulfonyl)-5- trifluoromethyl- benzenesulfonyl]- amino}-5,6,7,8- tetrahydro- naphthalen-1- yloxy)-acetic acid | 8.61 (s, 1 H), 8.55 (s, 1 H), 8.47 (s, 1 H), 7.10 (t, 1 H), 6.73 (dd, 2 H), 5.30 (t, 1 H), 4.66 (s, 2 H), 3.53-3.63 (m, 1H), 2.85 (d, J = 2.53 Hz, 1 H), 2.65 (s, 3 H), 2.54 (m, 1 H), 1.70 (m, 4 H), 1.32 (m, 6 H) | 550 | 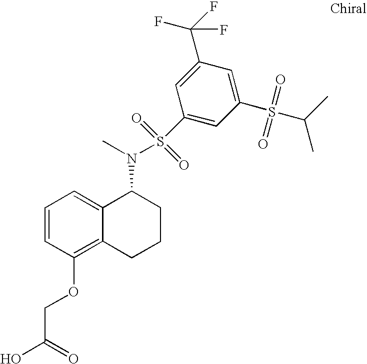 |
| 3-3 | ((R)-5-{methyl- [3-(2-methyl- propane-2- sulfonyl)-5- trifluoromethyl- benzenesulfonyl]- amino}-5,6,7,8- tetrahydro- naphthalen-1- yloxy)-acetic acid | 8.56 (s, 1 H), 8.55 (s, 1 H), 8.40 (s, 1 H), 7.08 (t, 1 H), 6.72 (dd, 2 H), 5.28 (t, 1 H), 4.67 (s, 2 H), 2.89 (d, 1 H), 2.64 (s, 3 H), 2.48- 2.59 (m, 2 H), 1.91- 2.00 (m, 1 H), 1.63-1.77 (m, 3 H), 1.35-1.39 (m, 9 H) | 564 | 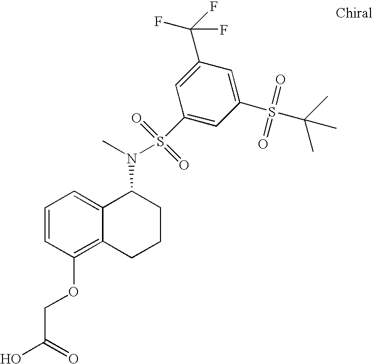 |

### Example 4-1

### {(R)-5-[methyl-(3-pyrrolidin-1-yl-5-trifluoromethyl-benzenesulfonyl)-amino]-5,6,7,8-tetrahydro-naphthalen-1-yloxy}-acetic acid

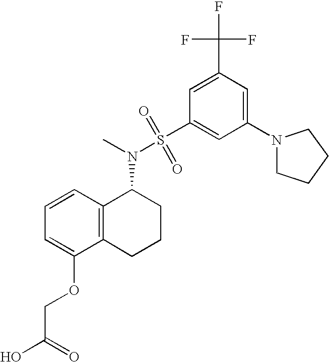

### {(R)-5-[methyl-(3-pyrrolidin-1-yl-5-trifluoromethyl-benzenesulfonyl)-amino]-5,6,7,8-tetrahydro-naphthalen-1-yloxy}-acetic acid tert-butyl ester

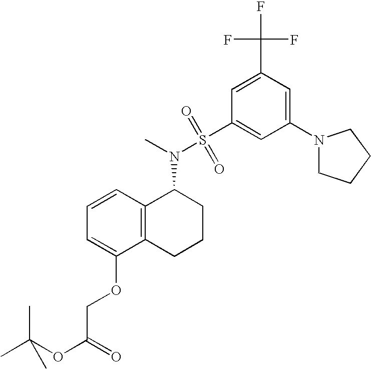

A mixture of {(R)-5-[(3-fluoro-5-trifluoromethyl-benzenesulfonyl)-methyl-amino]-5,6,7,8-tetrahydro-naphthalen-1-yloxy}-acetic acid tert-butyl ester (example 1-1, 5^{th} step) (87 mg, 0.168 mmol) and pyrrolidine (142 mg, 1.68 mmol)) in dimethyl sulfoxide (2 mL) was heated at 150° C. for 50 minutes in a microwave oven. To the reaction mixture was added water, and the resulting solution was extracted three times with ethyl acetate. The combined organic layers were dried over sodium sulfate, filtered, and then concentrated to afford {(R)-5-[methyl-(3-pyrrolidin-1-yl-5-trifluoromethyl-benzenesulfonyl)-amino]-5,6,7,8-tetrahydro-naphthalen-1-yloxy}-acetic acid tert-butyl ester, which was used in the next step without purification. MS cald. for C₂₈H₃₅F₃N₂O₅S 568, obsd 569 (ESI⁺) [(M+H)⁺]

### {(R)-5-[methyl-(3-pyrrolidin-1-yl-5-trifluoromethyl-benzenesulfonyl)-amino]-5,6,7,8-tetrahydro-naphthalen-1-yloxy}-acetic acid

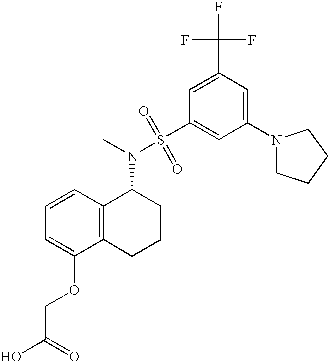

Starting with {(R)-5-[methyl-(3-pyrrolidin-1-yl-5-trifluoromethyl-benzenesulfonyl)-amino]-5,6,7,8-tetrahydro-naphthalen-1-yloxy}-acetic acid tert-butyl ester, and using the method analogous to the one described for example 1-1, {(R)-5-[methyl-(3-pyrrolidin-1-yl-5-trifluoromethyl-benzenesulfonyl)-amino]-5,6,7,8-tetrahydro-naphthalen-1-yloxy}-acetic acid (37 mg, 41% over two steps) was obtained as a white solid. HRMS cald. for C₂₄H₂₇F₃N₂O₅S (ESI⁺)[(M+Na)⁺] 535.1485, obsd. 535.1481; ¹H NMR (300 MHz, DMSO-d₆) δ ppm 12.21 (br. s, 1H), 7.21 (s, 1H), 7.06-7.15 (m, 2H), 7.00 (s, 1H), 6.74 (d, J=7.8 Hz, 1H), 6.70 (d, J=8.2 Hz, 1H), 5.06-5.18 (m, 1H), 4.66 (s, 2H), 3.35 (br. s, 4H), 2.64-2.82 (m, 1H), 2.51 (s, 3H), 2.41 (br. s, 1H), 2.00 (br. s, 4H), 1.83 (br. s, 1H), 1.42-1.75 (m, 3H).

### Example 4-2

### {(R)-5-[(3-diethylamino-5-trifluoromethyl-benzenesulfonyl)-methyl-amino]-5,6,7,8-tetrahydro-naphthalen-1-yloxy}-acetic acid

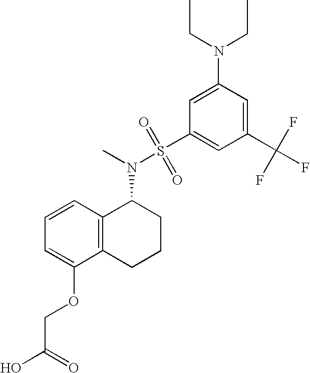

### {(R)-5-[(3-diethylamino-5-trifluoromethyl-benzenesulfonyl)-methyl-amino]-5,6,7,8-tetrahydro-naphthalen-1-yloxy}-acetic acid tert-butyl ester

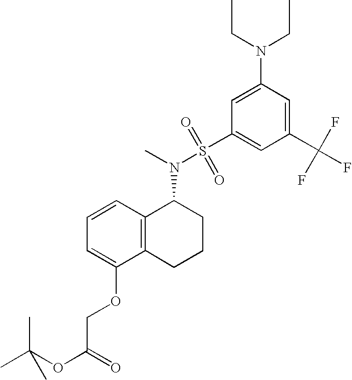

A mixture of {(R)-5-[(3-fluoro-5-trifluoromethyl-benzenesulfonyl)-methyl-amino]-5,6,7,8-tetrahydro-naphthalen-1-yloxy}-acetic acid tert-butyl ester (example 1-1, 5^{th} step) (87 mg, 0.168 mmol), sodium hydride (60% wt) (34 mg, 0.84 mmol) and diethylamine (175 μL, 1.68 mmol) in N,N-dimethylformamide (2 mL) was heated at 150° C. in a microwave oven for 45 minutes. To the reaction mixture was added water (10 mL), and the resulting solution was extracted with ethyl acetate (3×20 mL). The combined organic layers were dried over sodium sulfate, filtered, and then concentrated to afford {(R)-5-[(3-diethylamino-5-trifluoromethyl-benzenesulfonyl)-methyl-amino]-5,6,7,8-tetrahydro-naphthalen-1-yloxy}-acetic acid tert-butyl ester, which was used in the next step without purification. MS cald. for C₂₈H₃₇F₃NO₆S 570, obsd 571 (ESI⁺) [(M+H)⁺].

### {(R)-5-[(3-diethylamino-5-trifluoromethyl-benzenesulfonyl)-methyl-amino]-5,6,7,8-tetrahydro-naphthalen-1-yloxy}-acetic acid

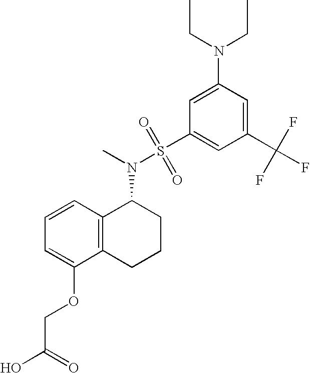

Starting with {(R)-5-[(3-diethylamino-5-trifluoromethyl-benzenesulfonyl)-methyl-amino]-5,6,7,8-tetrahydro-naphthalen-1-yloxy}-acetic acid tert-butyl ester, using the method analogous to the one described for example 1-1, {(R)-5-[(3-diethylamino-5-trifluoromethyl-benzenesulfonyl)-methyl-amino]-5,6,7,8-tetrahydro-naphthalen-1-yloxy}-acetic acid (11 mg, 13% over two steps) was obtained as a light brown solid. HRMS cald. for C₂₄H₂₉F₃NO₆S (ESI⁺)[(M+H)⁺] 515.1822, obsd. 515.1820; ¹H NMR (300 MHz, DMSO-d₆) δ ppm 7.20 (br. s, 1H), 7.18 (br. s, 1H), 7.11 (br. s, 1H), 7.05-7.10 (m, 1H), 6.71 (d, J=7.2 Hz, 1), 6.70 (d, J=8.2 Hz, 1H), 5.11 (br. s, 1H), 4.66 (s, 2H), 3.47 (q, J=6.9 Hz, 4H), 2.67 (d, J=14.8 Hz, 1H), 2.45 (br. s, 1H), 1.91 (s, 3H), 1.77 (br. s, 1H), 1.56-1.74 (m, 3H), 1.11 (t, J=6.9 Hz, 6H).

### Example 4-3

The following example 4-3 was prepared in an analogous manner as described for example 4-2 using {(R)-5-[(3-fluoro-5-trifluoromethyl-benzenesulfonyl)-methyl-amino]-5,6,7,8-tetrahydro-naphthalen-1-yloxy}-acetic acid tert-butyl ester and the commercially available N-methylisopropylamine. The final product was purified by reverse phase preparative HPLC.

| | | | MS | |
|---|---|---|---|---|
| Example | Systematic | ¹H NMR (DMSO-d₆) | (ESI+, | |
| No. | Name | δppm | M + H⁺) | Structure |
| 4-3 | ((R)-5-{[3-(isopropyl- methyl-amino)-5- trifluoromethyl- benzenesulfonyl]- methyl-amino}-5,6,7,8- tetrahydro-naphthalen- 1-yloxy)-acetic acid | 7.34 (s, 1 H), 7.23 (br. s, 2 H), 7.06 (t, J = 7.9 Hz, 1 H), 6.62- 6.71 (m, 2 H), 5.07- 5.18 (m, 1 H), 4.51 (br. s, 2 H), 4.26 (dt, J = 13.0, 6.5 Hz, 1 H), 2.81 (s, 3 H), 2.29- 2.46 (m, 2 H), 1.82 (br. s, 1 H), 1.52- 1.72 (m, 2 H), 1.50 (br. s, 1 H), 1.32 (s, 3 H), 1.15 (d, J = 6.4 Hz, 3 H), 1.15 (d, J = 6.4 Hz, 3 H) | 515 | 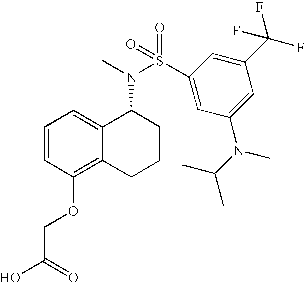 |

### Example 5-1

### ((R)-5-{[3-(1,1-difluoro-ethyl)-5-trifluoromethyl-benzenesulfonyl]-methyl-amino}-5,6,7,8-tetrahydro-naphthalen-1-yloxy)-acetic acid

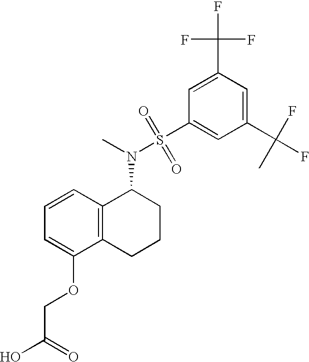

### [(R)-5-(3-bromo-5-trifluoromethyl-benzenesulfonylamino)-5,6,7,8-tetrahydro-naphthalen-1-yloxy]-acetic acid tert-butyl ester

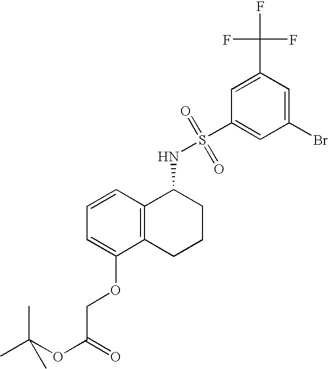

Starting with ((R)-5-amino-5,6,7,8-tetrahydro-naphthalen-1-yloxy)-acetic acid tert-butyl ester hydrochloride salt (500, 1.91 mmol) and 3-bromo-5-trifluoromethyl-benzenesulfonyl chloride (642 mg, 1.99 mmol), and using the method analogous to the one described for example 1-1, 4^{th} step, [(R)-5-(3-bromo-5-trifluoromethyl-benzenesulfonyl amino)-5,6,7,8-tetrahydro-naphthalen-1-yloxy]-acetic acid tert-butyl ester (711 mg, 66%) was obtained as a white solid. MS cald. for C₁₈H₁₉BrF₃N₃O₄S 564, obsd. 565 (ESI⁺) [(M+H)⁺].

### {(R)-5-[(3-bromo-5-trifluoromethyl-benzenesulfonyl)-methyl-amino]-5,6,7,8-tetrahydro-naphthalen-1-yloxy}-acetic acid tert-butyl ester

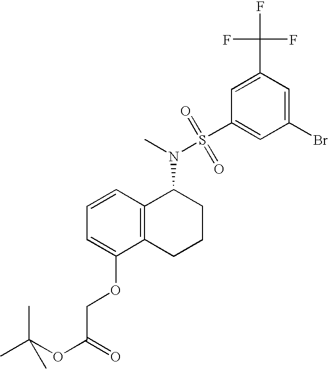

To a solution of [(R)-5-(3-bromo-5-trifluoromethyl-benzenesulfonylamino)-5,6,7,8-tetrahydro-naphthalen-1-yloxy]-acetic acid tert-butyl ester (46 mg, 0.08 mmol) in acetonitrile (3 mL) was added potassium carbonate (27.6 mg, 0.200 mmol) and methyl iodide (9.5 μL, 0.150 mmol) at room temperature. After being heated at 70° C. for 6 hours under an argon atmosphere, the reaction mixture was cooled to room temperature, filtered through a glass funnel, and concentrated in vacuo. The residue was purified by column chromatography (0-5% methanol in dichloromethane) to afford {(R)-5-[(3-bromo-5-trifluoromethyl-benzenesulfonyl)-methyl-amino]-5,6,7,8-tetrahydro-naphthalen-1-yloxy}-acetic acid tert-butyl ester (39 mg, 83%) as a white solid. MS cald. for C₂₀H₂₁F₆N₃O₄S 577, obsd. 578 (ESI⁺) [(M+H)⁺].

### {(R)-5-[(3-acetyl-5-trifluoromethyl-benzenesulfonyl)-methyl-amino]-5,6,7,8-tetrahydro-naphthalen-1-yloxy}-acetic acid tert-butyl ester

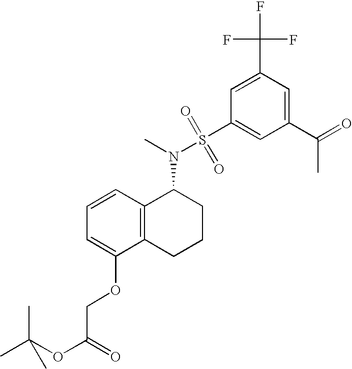

To a solution of {(R)-5-[(3-bromo-5-trifluoromethyl-benzenesulfonyl)-methyl-amino]-5,6,7,8-tetrahydro-naphthalen-1-yloxy}-acetic acid tert-butyl ester (1.0 g, 1.7 mmol) in N,N-dimethylformamide (8 mL) was added tris(dibenzylideneacetone)dipalladium(0) (175 mg, 0.19 mmol), triphenylarsine (175 mg, 0.57 mmol) and 1-ethoxy-vinyltributyltin (1 mL, 2.86 mmol) at room temperature. After being heated at 80° C. for 2 hours under an argon atmosphere, the reaction mixture was cooled to room temperature, and then treated with 4N hydrochloric acid (1 mL), and subsequently stirred at room temperature for 20 minutes. The resulting mixture was poured into water (40 mL) and extracted with ethyl acetate (20 mL×3). The combined organic layers were washed with water (20 mL), then brine (20 mL), and concentrated in vacuo. The residue was purified by flash column chromatography (15-30% ethyl acetate in petroleum ether) to afford {(R)-5-[(3-acetyl-5-trifluoromethyl-benzenesulfonyl)-methyl-amino]-5,6,7,8-tetrahydro-naphthalen-1-yloxy}-acetic acid tert-butyl ester (798 mg, 85%) as a yellow oil. MS cald. for C₂₁H₂₄F₃N₃O₅S 541, obsd. 542 (ESI⁺) [(M+H)⁺].

### ((R)-5-{[3-(1,1-difluoro-ethyl)-5-trifluoromethyl-benzenesulfonyl]-methyl-amino}-5,6,7,8-tetrahydro-naphthalen-1-yloxy)-acetic acid tert-butyl ester

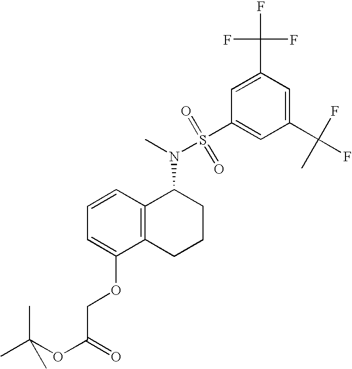

To a solution of {(R)-5-[(3-acetyl-5-trifluoromethyl-benzenesulfonyl)-methyl-amino]-5,6,7,8-tetrahydro-naphthalen-1-yloxy}-acetic acid tert-butyl ester (300 mg, 0.554 mmol) in anhydrous dichloromethane (3 mL) was added bis(2-methoxy-ethyl)aminosulfur trifluoride (400 μL, 2.17 mmol) at room temperature under an argon atmosphere. After being heated at 70° C. for 4 hours, the mixture was cooled to room temperature, and poured into saturated sodium bicarbonate and then extracted with dichloromethane (20 mL×3). The combined organic layers were washed with water (20 mL) and brine (20 mL), and then concentrated in vacuo. The residue was purified by flash column (15-30% ethyl acetate in petroleum ether) to afford ((R)-5-{[3-(1,1-difluoro-ethyl)-5-trifluoromethyl-benzenesulfonyl]-methyl-amino}-5,6,7,8-tetrahydro-naphthalen-1-yloxy)-acetic acid tert-butyl ester (250 mg, 80%) as a yellow oil. MS cald. for C₂₁H₂₄F₅N₃O₄S 563, obsd. 564 (ESI⁺) [(M+H)⁺].

### ((R)-5-{[3-(1,1-difluoro-ethyl)-5-trifluoromethyl-benzenesulfonyl]-methyl-amino}-5,6,7,8-tetrahydro-naphthalen-1-yloxy)-acetic acid

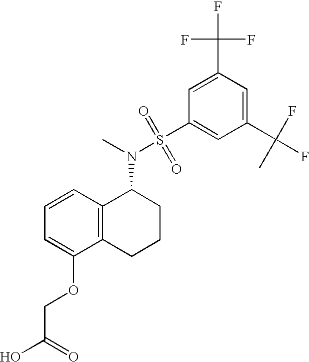

Starting with ((R)-5-{[3-(1,1-difluoro-ethyl)-5-trifluoromethyl-benzenesulfonyl]-methyl-amino}-5,6,7,8-tetrahydro-naphthalen-1-yloxy)-acetic acid tert-butyl ester (200 mg, 0.35 mmol), and using the method analogous to the one described for example 1-1, ((R)-5-{[3-(1,1-difluoro-ethyl)-5-trifluoromethyl-benzenesulfonyl]-methyl-amino}-5,6,7,8-tetrahydro-naphthalen-1-yloxy)-acetic acid (80 mg, 45%) was obtained as a white solid. MS cald. for C₁₅H₁₆F₅N₃O₄S 507, obsd. 508 (ESI⁺) [(M+H)⁺]; ¹H NMR (400 MHz, CD₃OD) δ ppm 8.28 (d, 2H), 8.14 (s, 1H), 7.07 (s, 1H), 6.75 (dd, 1H), 6.69 (d, 1H), 5.25 (t, 1H), 4.65 (s, 2H), 2.72-2.86 (d, 1H), 2.59 (s, 3H), 2.52 (m, 1H), 2.03 (t, 3H), 1.93 (m, 1H), 1.65 (m, 3H).

### Example 6-1

### {(R)-5-[(3-acetyl-5-trifluoromethyl-benzenesulfonyl)-methyl-amino]-5,6,7,8-tetrahydro-naphthalen-1-yloxy}-acetic acid

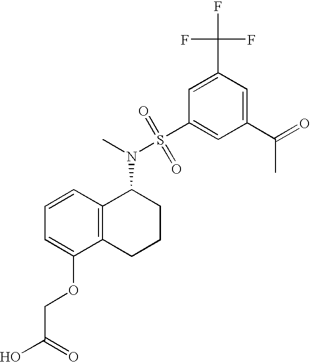

Starting with {(R)-5-[(3-acetyl-5-trifluoromethyl-benzenesulfonyl)-methyl-amino]-5,6,7,8-tetrahydro-naphthalen-1-yloxy}-acetic acid tert-butyl ester (example 5-1, 3^{rd} step) (230 mg, 0.42 mmol), and using the method analogous to the one described for example 1-1, {(R)-5-[(3-acetyl-5-trifluoromethyl-benzenesulfonyl)-methyl-amino]-5,6,7,8-tetrahydro-naphthalen-1-yloxy}-acetic acid (51.4 mg, 25%) was obtained as a white solid. HRMS cald. for C₂₂H₂₂F₃NO₆S (ESI⁺) [(M+Na)⁺] 508.1012, obsd. 508.1012; ¹H NMR (300 MHz, DMSO-d₆) δ ppm 8.56 (s, 1H), 8.52 (s, 1H), 8.44 (s, 1H), 7.06 (t, J=7.8 Hz, 1H), 6.60-6.68 (m, 2H), 5.17-5.29 (m, 1H), 4.42 (br. s., 2H), 2.75 (s, 3H), 2.68 (br. s, 1H), 2.55 (s, 3H), 2.21-2.46 (m, 1H), 1.81 (br. s, 1H), 1.53-1.73 (m, 2H), 1.50 (br. s, 1H).

### Example 7-1

### ((R)-5-{methyl-[3-(1-methyl-cyclopropyl)-5-trifluoromethyl-benzenesulfonyl]-amino}-5,6,7,8-tetrahydro-naphthalen-1-yloxy)-acetic acid

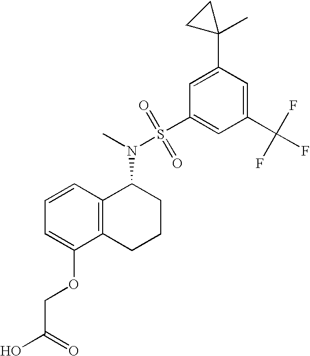

### [(R)-5-(3-isopropenyl-5-trifluoromethyl-benzenesulfonylamino)-5,6,7,8-tetrahydro-naphthalen-1-yloxy]-acetic acid tert-butyl ester

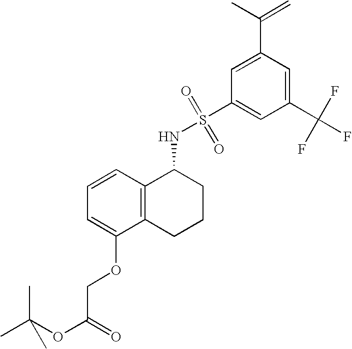

To a solution of [(R)-5-(3-bromo-5-trifluoromethyl-benzenesulfonylamino)-5,6,7,8-tetrahydro-naphthalen-1-yloxy]-acetic acid tert-butyl ester (example 5-1, 1^{st} step) (100 mg, 0.177 mmol) in N,N-dimethylformamide (1 mL) in a Biotage microwave vial were successively added tetrakis(triphenylphosphine)palladium(0) (21 mg, 0.0177 mmol), potassium tert-butoxide (40 mg, 0.35 mmol) and isopropenyl boronic acid pinacol ester (0.05 mL, 0.27 mmol). The resulting mixture was heated in a microwave at 130° C. for 15 minutes. After being cooled to room temperature, the reaction mixture was partitioned between 0.1 N hydrochloric acid and dichloromethane. The organic phase was extracted with water. The combined organic layers were dried over magnesium sulfate, filtered and concentrated under reduced pressure. Flash chromatography (RediSep® Flash column from Teledyne Isco, Inc., 230-400 mesh, 0-10% methanol in dichloromethane) gave [(R)-5-(3-isopropenyl-5-trifluoromethyl-benzenesulfonylamino)-5,6,7,8-tetrahydro-naphthalen-1-yloxy]-acetic acid tert-butyl ester (50 mg, 54%). MS cald. for C₂₆H₃₀F₃NO₅S 525, obsd. 526 (ESI⁺) [(M+H)⁺].

### [(R)-5-(3-isopropenyl-5-trifluoromethyl-benzenesulfonylamino)-5,6,7,8-tetrahydro-naphthalen-1-yloxy]-acetic acid

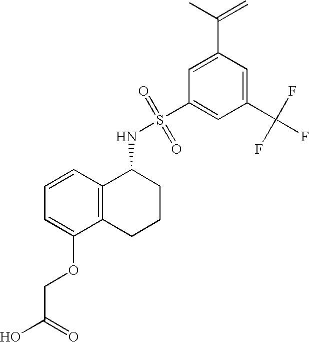

To a solution of the crude [(R)-5-(3-isopropenyl-5-trifluoromethyl-benzenesulfonylamino)-5,6,7,8-tetrahydro-naphthalen-1-yloxy]-acetic acid tert-butyl ester (326 mg, 0.62 mmol) in tetrahydrofuran (4 mL) was added 1M lithium hydroxide (4 mL). The resulting biphasic mixture was stirred at room temperature for 3 hours. The aqueous phase was washed with ethyl acetate, and then acidified with 1M HCl to a pH of about 2. The resulting mixture was extracted with ethyl acetate. The combined organic layers were concentrated to dryness under reduced pressure to give [(R)-5-(3-isopropenyl-5-trifluoromethyl-benzenesulfonylamino)-5,6,7,8-tetrahydro-naphthalen-1-yloxy]-acetic acid, which was used without further purification. MS cald for C₂₂H₂₂F₃NO₅S 469, obsd. 470 (ESI⁺) [(M+H)⁺].

### [(R)-5-(3-isopropenyl-5-trifluoromethyl-benzenesulfonylamino)-5,6,7,8-tetrahydro-naphthalen-1-yloxy]-acetic acid methyl ester

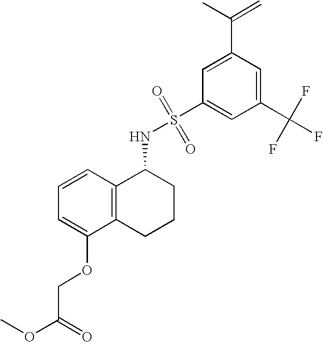

To a solution of the crude [(R)-5-(3-isopropenyl-5-trifluoromethyl-benzenesulfonylamino)-5,6,7,8-tetrahydro-naphthalen-1-yloxy]-acetic acid (60 mg, 011 mmol) in methanol (2 mL) was added a catalytic amount of thionyl chloride. The resulting reaction solution was heated in a microwave at 100° C. for 15 minutes. The mixture was concentrated to dryness to give crude [(R)-5-(3-isopropenyl-5-trifluoromethyl-benzenesulfonylamino)-5,6,7,8-tetrahydro-naphthalen-1-yloxy]-acetic acid methyl ester, which was used in the next step without further purification. MS cald for C₂₃H₂₄F₃NO₅S 483, obsd. 484 (ESI⁺) [(M+H)⁺].

### {(R)-5-[(3-isopropenyl-5-trifluoromethyl-benzenesulfonyl)-methyl-amino]-5,6,7,8-tetrahydro-naphthalen-1-yloxy}-acetic acid methyl ester

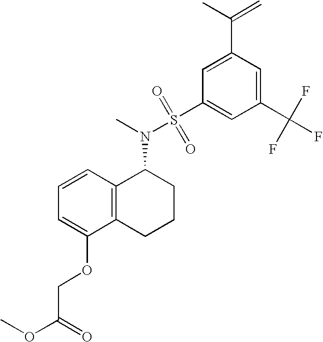

To a solution of [(R)-5-(3-isopropenyl-5-trifluoromethyl-benzenesulfonylamino)-5,6,7,8-tetrahydro-naphthalen-1-yloxy]-acetic acid methyl ester (67 mg, 0.14 mmol) in N,N-dimethylformamide (1 mL) were added potassium carbonate (48 mg, 0.345 mmol) and iodomethane (0.02 mL, 0.276 mmol). The mixture was heated at 100° C. in a microwave for 15 minutes. The mixture was partitioned between water and diethyl ether. The organic phase was washed 5 times with water, then concentrated to dryness to give crude {(R)-5-[(3-isopropenyl-5-trifluoromethyl-benzenesulfonyl)-methyl-amino]-5,6,7,8-tetrahydro-naphthalen-1-yloxy}-acetic acid methyl ester, which was used in the next step without further purification. MS cald. for C₂₄H₂₆F₃NO₅S 497, obsd. 498 (ESI⁺) [(M+H)⁺].

### ((R)-5-{methyl-[3-(1-methyl-cyclopropyl)-5-trifluoromethyl-benzenesulfonyl]-methyl-amino}-5,6,7,8-tetrahydro-naphthalen-1-yloxy)-acetic acid methyl ester

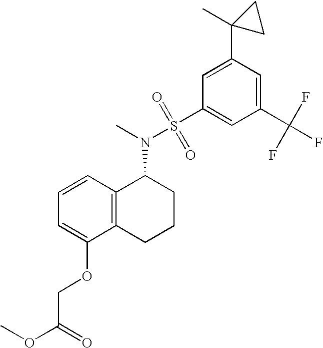

N-Nitroso-N-methylurea (600 mg, 5.83 mmol) was added in portions to a mixture of ether (10 mL) and 40% aqueous potassium hydroxide (2 mL) at 0° C. After 20 minutes, the aqueous layer was removed, and the ether layer was transferred via cannula to {(R)-5-[(3-isopropenyl-5-trifluoromethyl-benzenesulfonyl)-methyl-amino]-5,6,7,8-tetrahydro-naphthalen-1-yloxy}-acetic acid methyl ester (40 mg, 0.08 mmol) at 0° C., followed by addition of palladium acetate (2 mg, 0.009 mmol). The reaction mixture was quenched with 5 mL of acetic acid, and then filtered through a short pad of Celite® (a diatomite filter). The filtrate was concentrated in vacuo to give crude {(R)-5-[methyl-[3-(1-methyl-cyclopropyl)-5-trifluoromethyl-benzenesulfonyl]-methyl-amino]-5,6,7,8-tetrahydro-naphthalen-1-yloxy]-acetic acid methyl ester, which was used in the next step without further purification. MS cald. for C₂₅H₂₈F₃NO₅S 511, obsd. 512 (ESI⁺) [(M+H)⁺].

### ((R)-5-{methyl-[3-(1-methyl-cyclopropyl)-5-trifluoromethyl-benzenesulfonyl]-amino}-5,6,7,8-tetrahydro-naphthalen-1-yloxy)-acetic acid

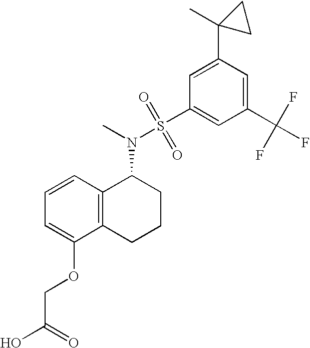

Starting with {(R)-5-[methyl-[3-(1-methyl-cyclopropyl)-5-trifluoromethyl-benzenesulfonyl]-methyl-amino]-5,6,7,8-tetrahydro-naphthalen-1-yloxy]-acetic acid methyl ester (16 mg, 0.032 mmol), using the method analogous to the one described for example 1-1, {(R)-5-[methyl-[3-(1-methyl-cyclopropyl)-5-trifluoromethyl-benzenesulfonyl]-methyl-amino]-5,6,7,8-tetrahydro-naphthalen-1-yloxy]-acetic acid (6 mg, 38%) was obtained as a solid. MS cald. for C₂₄H₂₆F₃NO₅S 497, obsd. 498 (ESI⁺) [(M+H)⁺]; ¹H NMR (400 MHz, DMSO-d₆) δ ppm 12.98 (br. s, 1H), 7.94 (s, 2H), 7.85 (s, 1H), 7.09 (t, J=8.0 Hz, 1H), 6.70 (d, J=8.0 Hz, 1H), 6.67 (d, J=8.0 Hz, 1H), 5.10-5.25 (m, 1H), 4.64 (s, 2H), 2.54 (s, 3H), 2.34-2.48 (m, 2H), 1.76-1.91 (m, 1H), 1.51-1.77 (m, 2H), 1.47 (s, 3H), 1.37-1.50 (m, 1H), 0.96-1.09 (m, 2H), 0.92 (d, 2H).

### Example 8-1

### {(R)-5-[(3-isopropyl-5-trifluoromethyl-benzenesulfonyl)-methyl-amino]-5,6,7,8-tetrahydro-naphthalen-1-yloxy}-acetic acid

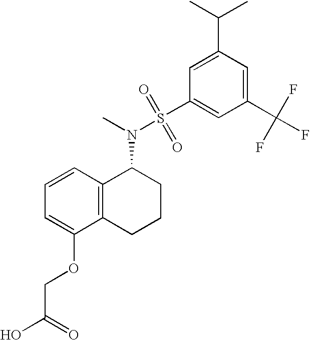

### {(R)-5-[(3-isopropenyl-5-trifluoromethyl-benzenesulfonyl)-methyl-amino]-5,6,7,8-tetrahydro-naphthalen-1-yloxy}-acetic acid tert-butyl ester

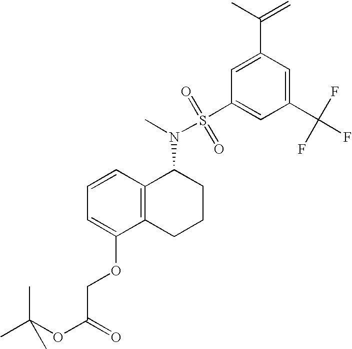

Starting with {(R)-5-[(3-bromo-5-trifluoromethyl-benzenesulfonyl)-methyl-amino]-5,6,7,8-tetrahydro-naphthalen-1-yloxy}-acetic acid tert-butyl ester (example 5-1, 2^{nd} step) (150 mg, 0.266 mmol) and isopropenyl boronic acid pinacol ester (0.075 mL, 0.40 mmol), using the method analogous to the one described for example 7-1, 1^{st} step, {(R)-5-[(3-isopropenyl-5-trifluoromethyl-benzenesulfonyl)-methyl-amino]-5,6,7,8-tetrahydro-naphthalen-1-yloxy}-acetic acid tert-butyl ester (52 mg, 36%) was obtained. MS cald. for C₂₇H₃₂F₃NO₅S 539, obsd. 540 (ESI⁺) [(M+H)⁺].

### {(R)-5-[(3-isopropyl-5-trifluoromethyl-benzenesulfonyl)-methyl-amino]-5,6,7,8-tetrahydro-naphthalen-1-yloxy}-acetic acid tert-butyl ester

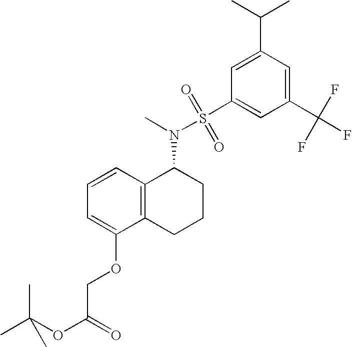

A mixture of {(R)-5-[(3-isopropenyl-5-trifluoromethyl-benzenesulfonyl)-methyl-amino]-5,6,7,8-tetrahydro-naphthalen-1-yloxy}-acetic acid tert-butyl ester (52 mg, 0.092 mmol) and 10% palladium on carbon (5 mg) in ethyl acetate (1.5 mL) in a CEM microwave vial was heated rapidly to 80° C. under hydrogen (50 psi) for 10 minutes. After being cooled to room temperature, the reaction mixture was filtered through a pad of Celite® (a diatomite filter), and washed with dichloromethane. The collected filtrate was concentrated under reduced pressure to give {(R)-5-[(3-isopropyl -5-trifluoromethyl-benzenesulfonyl)-methyl-amino]-5,6,7,8-tetrahydro-naphthalen-1-yloxy}-acetic acid tert-butyl ester (30 mg), which was used in the next step without further purification. MS cald. for C₂₇H₃₄F₃NO₅S 541, obsd. 542 (ESI⁺) [(M+H)⁺].

### {(R)-5-[(3-isopropyl-5-trifluoromethyl-benzenesulfonyl)-methyl-amino]-5,6,7,8-tetrahydro-naphthalen-1-yloxy}-acetic acid

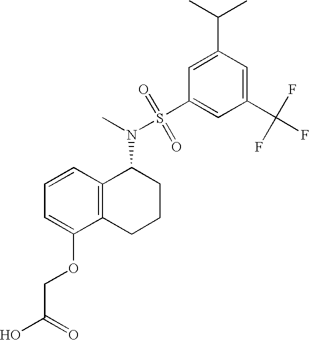

Starting with {(R)-5-[(3-isopropyl-5-trifluoromethyl-benzenesulfonyl)-methyl-amino]-5,6,7,8-tetrahydro-naphthalen-1-yloxy}-acetic acid tert-butyl ester (30 mg, 0.053 mmol), using the method analogous to the one described for example 7-1, 2^{nd} step, {(R)-5-[(3-isopropyl-5-trifluoromethyl-benzenesulfonyl)-methyl-amino]-5,6,7,8-tetrahydro-naphthalen-1-yloxy}-acetic acid (5 mg, 11% over two steps) was obtained as an oil. MS cald. for C₂₃H₃₆F₃NO₅S 485, obsd. 486 (ESI⁺) [(M+H)⁺]. ¹H NMR (300 MHz, CDCl₃) δ ppm 7.98 (s, 1H), 7.94 (s, 1H), 7.70 (s, 1H), 7.10 (t, J=7.8 Hz, 1H), 6.89 (d, J=7.8 Hz, 1H), 6.61 (d, J=7.8 Hz, 1H), 5.16-5.29 (m, 1H), 4.68 (s, 2H), 3.02-3.18 (m, 1H), 2.79-2.95 (m, 1H), 2.60 (s, 3H), 2.42-2.58 (m, 1H), 1.90-2.04 (m, 1H), 1.58-1.80 (m, 3H), 1.33 (d, 6H).

### Example 9-1

### {(R)-5-[(3-isopropoxy-5-trifluoromethyl-benzenesulfonyl)-methyl-amino]-5,6,7,8-tetrahydro-naphthalen-1-yloxy}-acetic acid

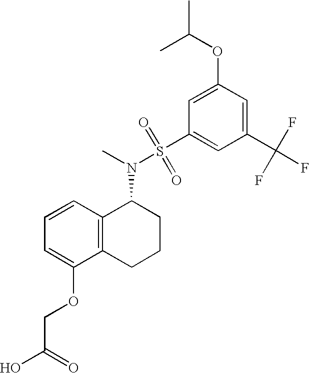

### {(R)-5-[(3-isopropoxy-5-trifluoromethyl-benzenesulfonyl)-methyl-amino]-5,6,7,8-tetrahydro-naphthalen-1-yloxy}-acetic acid tert-butyl ester

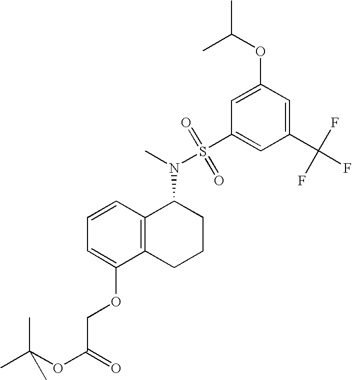

A mixture of {(R)-5-[(3-fluoro-5-trifluoromethyl-benzenesulfonyl)-methyl-amino]-5,6,7,8-tetrahydro-naphthalen-1-yloxy}-acetic acid tert-butyl ester (example 1-1, 5^{th} step) (87 mg, 0.168 mmol), sodium hydride (60% wt) (34 mg, 0.84 mmol) and 2-propanol (110 μL, 1.83 mmol) in N,N-dimethylformamide (2 mL) was heated at 150° C. in a microwave oven for 45 minutes. To the reaction mixture was added water (10 mL), and the resulting solution was extracted with ethyl acetate (3×20 mL). The combined organic layers were dried over sodium sulfate, filtered, and then concentrated to afford {(R)-5-[(3-isopropoxy-5-trifluoromethyl-benzenesulfonyl)-methyl-amino]-5,6,7,8-tetrahydro-naphthalen-1-yloxy}-acetic acid tert-butyl ester, which was used in the next step without purification. MS cald. for C₂₇H₃₄F₃NO₆S 557, obsd 558 (ESI⁺) [(M+H)⁺].

### {(R)-5-[(3-isopropoxy-5-trifluoromethyl-benzenesulfonyl)-methyl-amino]-5,6,7,8-tetrahydro-naphthalen-1-yloxy}-acetic acid

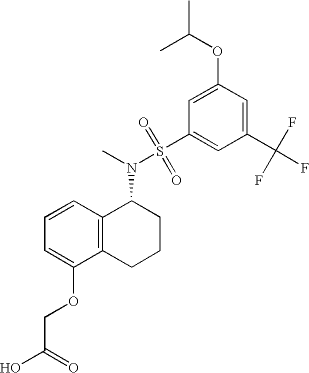

Starting with {(R)-5-[(3-isopropoxy-5-trifluoromethyl-benzenesulfonyl)-methyl-amino]-5,6,7,8-tetrahydro-naphthalen-1-yloxy}-acetic acid tert-butyl ester, using the method analogous to the one described for example 1-1, {(R)-5-[(3-isopropoxy-5-trifluoromethyl-benzenesulfonyl)-methyl-amino]-5,6,7,8-tetrahydro-naphthalen-1-yloxy}-acetic acid (15 mg, 18% over two steps) was obtained as a white solid. HRMS cald. for C₂₃H₂₆F₃NO₆S (ESI⁺)[(M+Na)⁺]524.1352, obsd. 524.1322; ¹H NMR (300 MHz, DMSO-d₆) δ ppm 12.49 (br. s, 1H), 7.66 (s, 1H), 7.64 (s, 1H), 7.59 (s, 1H), 7.09 (t, J=8.2 Hz, 1H), 6.70 (d, J=8.2 Hz, 1H), 6.66 (d, J=8.2 Hz, 1H), 5.11-5.23 (m, 1H), 4.86-5.00 (m, 1H), 4.66 (s, 2H), 2.67-2.79 (m, 1H), 2.53 (s, 3H), 2.32-2.46 (m, 1H), 1.75-1.89 (m, 1H), 1.58-1.74 (m, 2H), 1.48 (br. s, 1H), 1.30 (d, J=5.7 Hz, 6H).

### Examples 9-2 and 9-3

The following examples 9-2 and 9-3 were prepared in an analogous manner as described for example 9-1, using {(R)-5-[(3-fluoro-5-trifluoromethyl-benzenesulfonyl)-methyl-amino]-5,6,7,8-tetrahydro-naphthalen-1-yloxy}-acetic acid tert-butyl ester and the appropriate commercially available alcohols.

| | | | MS | |
|---|---|---|---|---|
| Example | Systematic | ¹H NMR (300 MHz,) | (ESI+, | |
| No. | Name | δ ppm | M + Na⁺) | Structure |
| 9-2* | {(R)-5-[(3-ethoxy- 5-trifluoromethyl- benzenesulfonyl)- methyl-amino]- 5,6,7,8- tetrahydro- naphthalen-1- yloxy}-acetic acid | (CDCl₃) 7.71 (br. s, 1 H), 7.56 (br. s, 1 H), 7.34 (br. s, 1 H), 7.11 (t, J = 7.8 Hz, 1 H), 6.90 (d, J = 7.8 Hz, 1 H), 6.62 (d, J = 7.8 Hz, 1 H), 5.13- 5.29 (m, 1 H), 4.68 (s, 2 H), 4.07-4.21 (m, 2 H), 2.82-3.03 (m, 1 H), 2.61 (s, 3 H), 2.41-2.59 (m, 1 H), 1.96 (br. s, 1 H), 1.58- 1.80 (m, 3 H), 1.48 (t, J = 6.8 Hz, 3 H) | 510 | 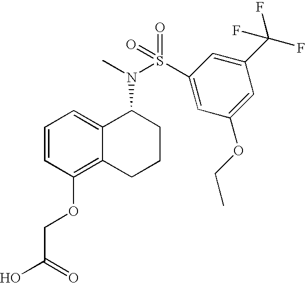 |
| 9-3 | {(R)-5-[(3- cyclopentyloxy-5- trifluoromethyl- benzenesulfonyl)- methyl-amino]- 5,6,7,8- tetrahydro- naphthalen-1- yloxy}-acetic acid | (DMSO-d₆) 13.24 (br. s, 1 H), 7.67 (s, 1 H), 7.61 (s, 1 H), 7.57 (s, 1 H), 7.08 (t, J = 8.5 Hz, 1 H), 6.69 (d, J = 8.5 Hz, 1 H), 6.66 (d, J = 8.5 Hz, 1 H), 5.04- 5.22 (m, 2 H), 4.63 (s, 2 H), 2.69 (br. s, 1 H), 2.53 (s, 3 H), 2.33- 2.45 (m, 1 H), 1.42- 2.07 (m, 12 H) | 550 | 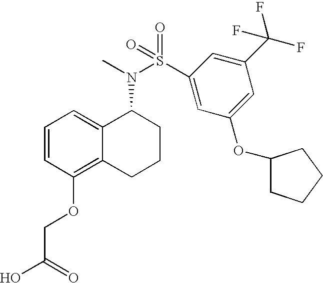 |

### Example 10-1

### [(R)-5-(3,5-dichloro-benzenesulfonylamino)-5,6,7,8-tetrahydro-naphthalen-1-yloxy]-acetic acid

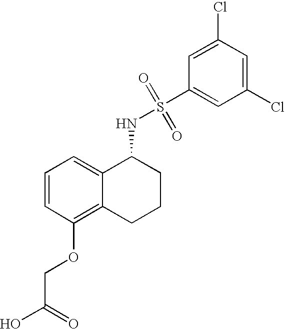

To a solution of ((R)-5-amino-5,6,7,8-tetrahydro-naphthalen-1-yloxy)-acetic acid tert-butyl ester hydrochloride salt (prepared as described above, 25 mg, 0.08 mmol) and N,N-diisopropylethylamine (0.022 mL, 0.14 mmol) in dry tetrahydrofuran (1 mL) was added 3,5-dichlorobenzenesulfonyl chloride (34 mg, 0.11 mmol) at room temperature. The reaction mixture was stirred at room temperature for 4 hours, at which time analysis of an aliquot by LC/MS showed complete consumption of the starting amine. To the reaction mixture was added 0.2 N lithium hydroxide (1 mL), and the resulting mixture was stirred overnight. Analysis showed only partial hydrolysis of the ester. Additional 0.2 N lithium hydroxide was then added (1 mL) and the mixture was stirred at room temperature for 2 days. The solution was acidified and concentrated to dryness. Preparative HPLC (Pursuit C-18,H₂O/CH₃CN/TFA) provided pure [(R)-5-(3,5-dichloro-benzenesulfonylamino)-5,6,7,8-tetrahydro-naphthalen-1-yloxy]-acetic acid (12 mg, 31%); ¹H NMR (400 MHz, DMSO-d₆) δ ppm 8.39 (d, J=8.3 Hz, 1H), 7.99 (t, J=1.7 Hz, 1H), 7.86 (d, J=1.7 Hz, 2H), 7.05 (t, J=8.0 Hz, 1), 6.68 (d, J=8.0 Hz, 1H), 6.62 (d, J=8.0 Hz, 1H), 4.63 (s, 2H), 4.40-4.52 (m, 1H), 2.53-2.64 (m, 2H), 1.70-1.86 (m, 1H), 1.50-1.69 (m, 3H). HRMS cald. for C₁₈H₁₇Cl₂NO₅S (ESI⁺)[(M+H)⁺]428.0131, obsd. 428.0130.

### Example 10-2

### {(R)-5-[3-(1,1-difluoro-ethyl)-5-trifluoromethyl-benzenesulfonylamino]-5,6,7,8-tetrahydro-naphthalen-1-yloxy}-acetic acid

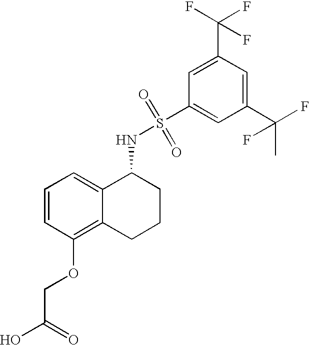

### {(R)-5-[benzyl-(3-bromo-5-trifluoromethyl-benzenesulfonyl)-amino]-5,6,7,8-tetrahydro-naphthalen-1-yloxy}-acetic acid tert-butyl ester

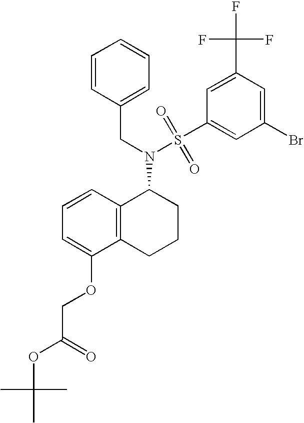

To a solution of (R)-[5-(3-bromo-5-trifluoromethyl-benzenesulfonylamino)-5,6,7,8-tetrahydro-naphthalen-1-yloxy]-acetic acid tert-butyl ester (50 mg, 0.088 mmol, prepared as described above) in acetonitrile (3 mL) was added potassium carbonate (27.6 mg, 0.200 mmol) and bromomethyl-benzene (45 mg, 0.265 mmol). The reaction mixture was heated at 70° C. for 6 hours under an argon atmosphere, and then cooled to room temperature, filtered through a glass funnel and concentrated in vacuo. The residue was purified by column chromatography (gradient elution, 0-5% methanol in dichloromethane) to afford {(R)-5-[benzyl-(3-bromo-5-trifluoromethyl-benzenesulfonyl)-amino]-5,6,7,8-tetrahydro-naphthalen-1-yloxy}-acetic acid tert-butyl ester (46 mg, 80%) as a white solid. MS cald. for C₃₀H₃₁BrF₃NO₅S 654, obsd. (ESI⁺) [(M+H)⁺] 655.

### {(R)-5-[(3-acetyl-5-trifluoromethyl-benzenesulfonyl)-benzyl-amino]-5,6,7,8-tetrahydro-naphthalen-1-yloxy}-acetic acid tert-butyl ester

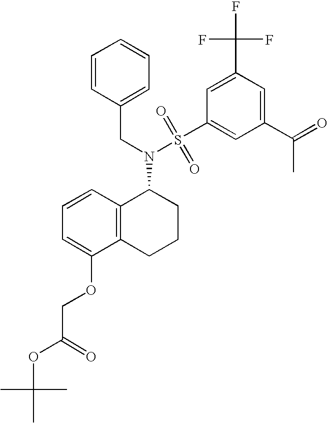

To a solution of {(R)-5-[benzyl-(3-bromo-5-trifluoromethyl-benzenesulfonyl)-amino]-5,6,7,8-tetrahydro-naphthalen-1-yloxy}-acetic acid tert-butyl ester (1.0 g, 1.53 mmol) in N,N-dimethylformamide (8 mL) was added tris(dibenzylideneacetone)dipalladium(0) (175 mg, 0.19 mmol), triphenylarsine (175 mg, 5.72 mmol), and 1-ethoxy-vinyltributyltin (1 mL, 2.86 mmol). After being stirred at 80° C. for 2 hours under an argon atmosphere, the reaction mixture was cooled to room temperature, and then treated with 4N hydrochloric acid (1 mL), and stirred at room temperature for 20 minutes. The resulting mixture was poured into water (40 mL) and extracted with ethyl acetate (3×20 mL). The combined organic layers were washed with water (20 mL) and brine (20 mL), then concentrated in vacuo. The residue was purified by flash column chromatography (gradient elution: 15-30% ethyl acetate in petroleum ether) to afford {(R)-5-[(3-acetyl-5-trifluoromethyl-benzenesulfonyl)-benzyl-amino]-5,6,7,8-tetrahydro-naphthalen-1-yloxy}-acetic acid tert-butyl ester as a yellow oil (815 mg, 86.4%). MS cald. for C₃₂H₃₄F₃NO₆S 617, obsd. (ESI⁺) [(M+H⁾⁺] 618.

### ((R)-5-{benzyl-[3-(1,1-difluoro-ethyl)-5-trifluoromethyl-benzenesulfonyl]-amino}-5,6,7,8-tetrahydro-naphthalen-1-yloxy)-acetic acid tert-butyl ester

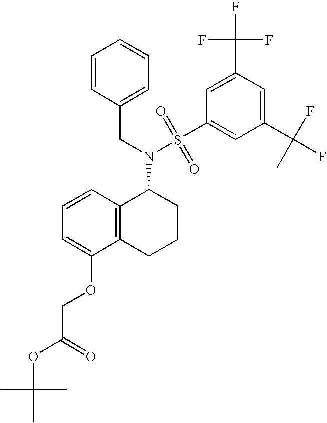

To a solution of {(R)-5-[(3-acetyl-5-trifluoromethyl-benzenesulfonyl)-benzyl-amino]-5,6,7,8-tetrahydro-naphthalen-1-yloxy}-acetic acid tert-butyl ester (300 mg, 0.486 mmol) in anhydrous dichloromethane (3 mL) in a bomb bottle (5 mL) was added bis(2-methoxy-ethyl)aminosulfur trifluoride (400 μL, 2.17 mmol) under an argon atmosphere. After being stirred at 70° C. for 4 hours, the mixture was cooled to room temperature, and poured into saturated sodium bicarbonate solution and extracted with dichloromethane (3×20 mL). The combined organic layers were washed with water (20 mL) and brine (20 mL), then concentrated in vacuo. The residue was purified by flash column chromatography (gradient elution: 15-30% ethyl acetate in petroleum ether) to afford ((R)-5-{benzyl-[3-(1,1-difluoro-ethyl)-5-trifluoromethyl-benzenesulfonyl]-amino}-5,6,7,8-tetrahydro-naphthalen-1-yloxy)-acetic acid tert-butyl ester (247 mg, 79.7%) as a yellow oil. MS cald for C₃₂H₃₁F₅NO₅S 639, obsd. (ESI+) [(M+H)⁺] 640.

### {(R)-5-[3-(1,1-difluoro-ethyl)-5-trifluoromethyl-benzenesulfonylamino]-5,6,7,8-tetrahydro-naphthalen-1-yloxy}-acetic acid tert-butyl ester

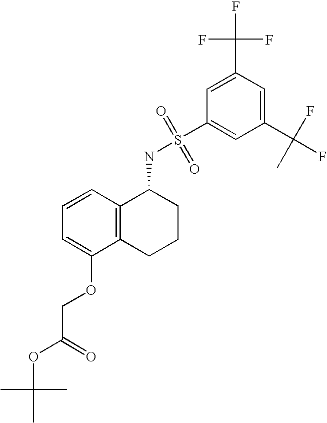

((R)-5-{benzyl-[3-(1,1-difluoro-ethyl)-5-trifluoromethyl-benzenesulfonyl]-amino}-5,6,7,8-tetrahydro-naphthalen-1-yloxy)-acetic acid tert-butyl ester (90 mg, 0.14 mmol), palladium on carbon (15 mg, 10% w/w), and formic acid ammonium salt (65 mg, 1.03 mmol) were suspended in ethanol (15 mL), and the resulting mixture was heated at 60° C. for 5 hours. The reaction mixture was then cooled to room temperature, and filtered through celite. The filtrate was washed with ethanol (3×10 mL), and the collected organic layers were concentrated in vacuo. The residue was purified by flash column chromatography (gradient elution: 15-30% ethyl acetate in petroleum ether) to afford {(R)-5-[3-(1,1-difluoro-ethyl)-5-trifluoromethyl-benzenesulfonylamino]-5,6,7,8-tetrahydro-naphthalen-1-yloxy}-acetic acid tert-butyl ester (46 mg, 60%). MS cald for C₂₅H₂₈F₅NO₅S 549, obsd. (ESI⁺) [(M+H)⁺] 550.

### {(R)-5-[3-(1,1-difluoro-ethyl)-5-trifluoromethyl-benzenesulfonyl]-methyl-amino}-5,6,7,8-tetrahydro-naphthalen-1-yloxy)-acetic acid

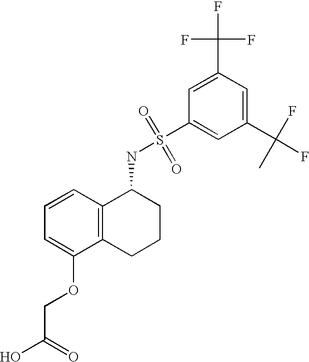

Starting with {(R)-5-[3-(1,1-difluoro-ethyl)-5-trifluoromethyl-benzenesulfonylamino]-5,6,7,8-tetrahydro-naphthalen-1-yloxy}-acetic acid tert-butyl ester, and using the method analogous to the one described for example 1-1, {(R)-5-[3-(1,1-difluoro-ethyl)-5-trifluoromethyl-benzenesulfonylamino]-5,6,7,8-tetrahydro-naphthalen-1-yloxy}-acetic acid was obtained as a white solid. ¹H NMR (400 MHz, CD₃OD) δ ppm 8.30 (s, 1H), 8.28 (s, 1H), 8.09 (s, 1H), 6.94 (dd, 1H), 6.64 (d, 1H), 6.46 (d, 2H), 4.63 (s, 2H), 4.46 (t, 1H), 2.72-2.83 (m, 1H), 2.51-2.63 (m, 1H), 2.01 (t, 3H), 1.65-1.88 (m, 4H), MS cald for C₂₁H₂₀F₅NO₅S 493, obsd. (ESI⁺) [(M+H)⁺]: 494.

### Examples 10-3 to 10-12

The following examples 10-3 to 10-5 and 10-8 to 10-12 were prepared in an analogous manner as described above for examples 1-1 and 10-1 by treating ((R)-5-amino-5,6,7,8-tetrahydro-naphthalen-1-yloxy)-acetic acid tert-butyl ester hydrochloride salt (VI, prepared as described in Schemes 1 or 2) with the appropriate substituted benzenesulfonyl chloride, followed by ester hydrolysis (without the methylation step using iodomethane). For examples 10-6 and 10-7, the compounds were prepared using the procedures described above for making examples 8-1 and 7-1, respectively (N-methylated derivatives), starting with the appropriate NH-sulfonamides without the methylation step using iodomethane.

| Example | Systematic | ¹H NMR (300 MHz,) | | |
|---|---|---|---|---|
| No. | Name | δ ppm | MS | Structure |
| 10-3 | [(R)-5-(3,5-bis- trifluoromethyl- benzenesulfonylamino)- 5,6,7,8-tetrahydro- naphthalen-1-yloxy]- acetic acid | (DMSO-d₆) 8.48-8.61 (m, 2 H), 8.46 (br. s, 2 H), 6.88-7.06 (m, 1H), 6.66 (d, J = 6.9 Hz, 1 H), 6.51 (d, J = 7.2 Hz, 1 H), 1 H), 4.54 (br. s, 3 H), 2.40-2.69 (m, 2 H), 1.68-1.86 (m, 1 H), 1.58 (br. s, 3 H) | 496^{#} | 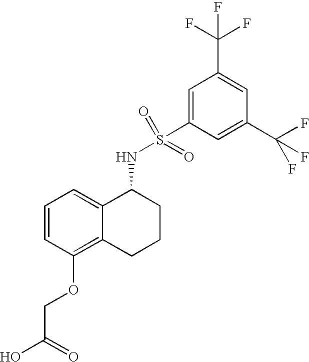 |
| 10-4 | [(R)-5-(3,5-dimethyl- benzenesulfonylamino)- 5,6,7,8-tetrahydro- naphthalen-1-yloxy]- acetic acid | (400 MHz, DMSO-d₆) 12.86 (br. s, 1 H), 7.98 (d, J = 8.5 Hz, 1 H), 7.49 (s, 2 H), 7.29 (s, 1 H), 7.02 (t, J = 8.0 Hz, 1 H), 6.66 (d, J = 8.1 Hz, 2 H), 4.63 (s, 2 H), 4.21-4.35 (m, 1 H), 2.42- 2.64 (m, 2 H), 2.37 (s, 6 H), 1.78 (br. s, 1 H), 1.44- 1.66 (m, 3 H) | 388^{#} | 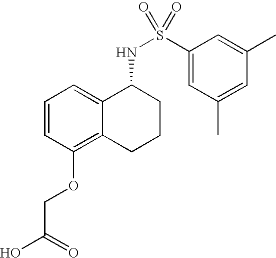 |
| 10-5 | [(R)-5-(3,5-difluoro- benzenesulfonylamino)- 5,6,7,8-tetrahydro- naphthalen-1-yloxy]- acetic acid | 12.93 (br. s, 1 H), 8.35 (d, J = 8.5 Hz, 1 H), 7.52- 7.69 (m, 3 H), 7.04 (t, J = 7.8 Hz, 1 H), 6.58-6.76 (m, 2 H), 4.64 (s, 2 H), 4.34-4.50 (m, 1 H), 2.51- 2.60 (m, 2 H), 1.67-1.86 (m, 1 H), 1.46-1.66 (m, 3 H) | 396^{#} | 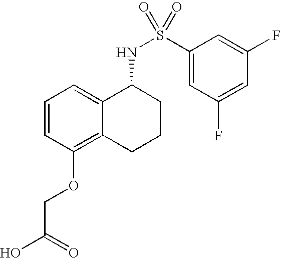 |
| 10-6 | [(R)-5-(3-isopropyl-5- trifluoromethyl- benzenesulfonylamino)- 5,6,7,8-tetrahydro- naphthalen-1-yloxy]- acetic acid | (400 MHz, DMSO-d₆) 12.87 (br. s, 1 H), 8.31 (d, J = 8.3 Hz, 1 H), 8.07 (s, 1 H), 7.96 (s, 1 H), 7.93 (s, 1 H), 6.99 (t, J = 8.0 Hz, 1 H), 6.67 (d, J = 8.0 Hz, 1 H), 6.54 (d, J = 8.0 Hz, 1 H), 4.63 (s, 2 H), 4.36- 4.46 (m, 1 H), 3.07-3.24 (m, J = 6.8 Hz, 1 H), 2.50- 2.65 (m, 2 H), 1.75 (br. s, 1 H), 1.42-1.68 (m, 3 H), 1.27 (d, J = 6.8 Hz, 6 H) | 470^{#} | 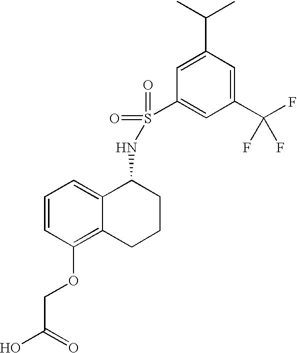 |
| 10-7 | {(R)-5-[3-(1-methyl- cyclopropyl)-5- trifluoromethyl- benzenesulfonylamino]- 5,6,7,8-tetrahydro- naphthalen-1-yloxy}- acetic acid | (CD₃OD) 8.01 (s, 1 H), 7.97 (s, 1 H), 7.78 (s, 1 H), 6.94 (t, J = 7.8 Hz, 1 H), 6.64 (d, J = 7.8 Hz, 1 H), 6.42 (d, J = 7.8 Hz, 1 H), 4.63 (s, 2 H), 4.33- 4.43 (m, 1 H), 2.71-2.86 (m, 1 H), 2.49-2.65 (m, 1 H), 1.60-1.95 (m, 4 H), 1.48 (s, 3 H), 0.98 (br. s, 2 H), 0.93 (br. s, 2 H) | N/A | 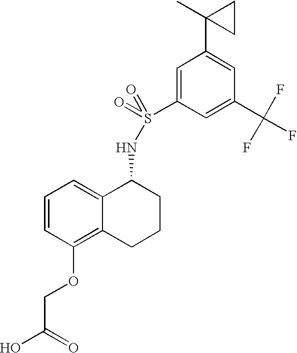 |
| 10-8 | [(R)-5-(3,5-di-tert- butyl- benzenesulfonylamino)- 5,6,7,8-tetrahydro- naphthalen-1-yloxy]- acetic acid | (CDCl₃) 7.77 (d, J = 1.2 Hz, 2 H), 7.62-7.71 (m, 1 H), 6.97 (t, J = 8.0 Hz, 1 H), 6.57 (d, J = 8.0 Hz, 1 H), 6.50 (d, J = 8.0 Hz, 1 H), 4.71 (d, J = 7.5 Hz, 1 H), 4.66 (s, 2 H), 4.43 (br. s, 1 H), 2.69-2.90 (m, 1 H), 2.47-2.64 (m, 1 H), 1.67-1.95 (m, 4 H), 1.37 (s, 18 H) | 474* | 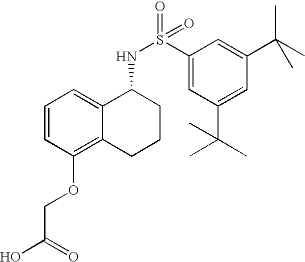 |
| 10-9 | [(R)-5-(3,5-bis- methanesulfonyl- benzenesulfonylamino)- 5,6,7,8-tetrahydro- naphthalen-1-yloxy]- acetic acid | (DMSO-d₆) 11.80-13.65 (br. s, 1 H), 8.65 (s, 3 H), 8.59 (d, J = 8.2 Hz, 1 H), 7.00 (t, J = 8.0 Hz, 1 H), 6.66 (d, J = 8.0 Hz, 1 H), 6.55 (d, J = 8.0 Hz, 1 H), 4.64 (s, 2 H), 4.43-4.57 (m, 1 H), 3.43 (s, 6 H), 2.46-2.67 (m, 2 H), 1.66- 1.87 (m, 1 H), 1.46-1.67 (m, 3 H) | 518* | 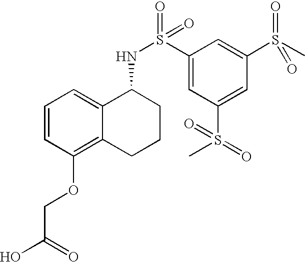 |
| 10-10 | [(R)-5-(3-methoxy-5- trifluoromethyl- benzenesulfonylamino)- 5,6,7,8-tetrahydro- naphthalen-1-yloxy]- acetic acid | (DMSO_d₆) 12.98 (br. s, 1 H), 8.35 (d, J = 8.2 Hz, 1 H), 7.71 (s, 1 H), 7.66 (s, 1 H), 7.57 (s, 1 H), 7.03 (t, J = 7.8 Hz, 1 H), 6.68 (d, J = 7.8 Hz, 1 H), 6.62 (d, J = 7.8 Hz, 1 H), 4.66 (s, 2 H), 4.31-4.50 (m, 1 H), 3.93 (s, 3 H), 2.52-2.66 (m, 2 H), 1.68-1.87 (m, 1 H), 1.46-1.67 (m, 3 H) | 482** | 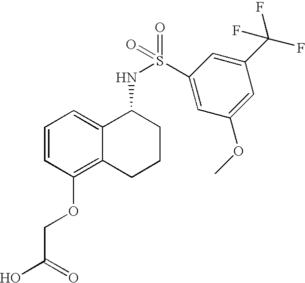 |
| 10-11 | [(R)-5-(3-bromo-5- trifluoromethyl- benzenesulfonylamino)- 5,6,7,8-tetrahydro- naphthalen-1-yloxy]- acetic acid | (DMSO-d₆) 12.98 (s, 1 H), 8.47 (d, J = 8.5 Hz, 1 H), 8.37 (s, 1 H), 8.31 (s, 1 H), 8.15 (s, 1 H), 7.03 (t, J = 7.8 Hz, 1 H), 6.69 (d, J = 7.8 Hz, 1 H), 6.58 (d, J = 7.8 Hz, 1 H), 4.66 (s, 2 H), 4.40-4.56 (m, 1 H), 2.54- 2.67 (m, 2 H), 1.69-1.89 (m, 1 H), 1.49-1.68 (m, 3 H) | 530** | 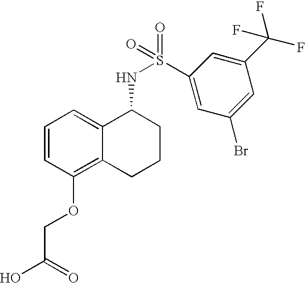 |
| 10-12 | [(R)-5-(3-fluoro-5- trifluoromethyl- benzenesulfonylamino)- 5,6,7,8-tetrahydro- naphthalen-1-yloxy]- acetic acid | (DMSO-d₆) 12.98 (s, 1 H), 8.47 (d, J = 8.5 Hz, 1 H), 8.11 (d, J = 8.5 Hz, 1 H), 7.97-8.08 (m, 2 H), 7.03 (t, J = 8.0 Hz, 1 H), 6.69 (d, J = 8.0 Hz, 1 H), 6.60 (d, J = 8.0 Hz, 1 H), 4.66 (s, 2 H), 4.35-4.59 (m, 1 H), 2.52-2.67 (m, 2 H), 1.68-1.86 (m, 1 H), 1.45- 1.69 (m, 3 H) | 470** | 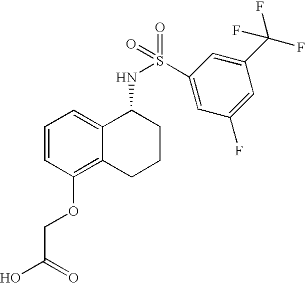 |

^{#}[M − H]⁻ observed
*[M + H]+ observed
**[M + Na]+ observed

### Activity and Use of the Compounds

The compounds of formula I possess valuable pharmacological properties. It has been found that said compounds are antagonists at the CRTH2 receptor and may be useful in treating diseases and disorders associated with that receptor such as asthma. The activity of the present compounds as CRTH2 receptor antagonists is demonstrated by the following biological assays.

### Human CRTH2 Receptor Binding Assay

A whole cell receptor binding assay using [³H]ramatroban as the competing radioactive ligand was employed to evaluate the compound binding activity to human CRTH2. The radioactive ligand [³H]ramatroban was synthesized according to Sugimoto et. al. (Eur. J. Pharmacol. 524, 30-37, 2005) to a specific activity of 42 Ci/mmol.

A cell line stably expressing human CRTH2 was established by transfecting CHO-K1 cells with two mammalian expression vectors that harbored human CRTH2 and G-alpha16 cDNAs, respectively, using FuGene® 6 transfection reagent (from Roche). Stable clones expressing CRTH2 were selected by staining each clone with BM16 (BD Pharmingen™ from BD Biosciences, a division of Becton, Dickinson and Company), which is a rat monoclonal antibody to human CRTH2. The cells were maintained as monolayer cultures in Ham's F-12 medium containing 10% fetal bovine serum, 100 units/mL penicillin, 100 μg/mL streptomycin, 2 mM glutamine, 0.5 mg/mL G418 (geneticin) for CRTH2, and 0.2 mg/mL hygromycin-B (for G-alpha 16). For whole cell receptor binding assay, the monolayer cells were rinsed once with PBS (phosphate buffered saline), dissociated using ethylenediaminetetraacetate (Versene™ EDTA from Lonza Inc.), and suspended in PBS containing 10 mM MgCl₂ and 0.06% BSA (bovine serum albumin) at 1.5×10⁶ cells/mL.

The binding reactions (0.2 mL) were performed in 96-well plates at room temperature in PBS containing 1.5×10⁵ cells, 10 mM MgCl₂, 0.06% BSA, 20 nM [³H]ramatroban, and test compound at various concentrations. After 1 hour of binding reactions, the cells were harvested on GF™/B filter microplates (microtiter plates with embedded glass fiber from PerkinElmer, Inc.) and washed 5 times with PBS using a Filtermate™ Harvester (a cell harvester that harvests and washes cells from microplates from PerkinElmer, Inc.). The radioactivities bound to the cells were determined using a microplate scintillation counter (TopCount® NXT, from PerkinElmer, Inc.) after adding 50 μL of Microscint™ 20 scintillation fluid (from PerkinElmer, Inc.) to each well of the filter plates. The radioactivity from non-specific binding was determined by replacing compound with 10 μM of 15(R)-15-methyl PGD₂ (from Cayman Chemical Company) in the reaction mixtures. The radioactivity bound to the cells in the absence of compound (total binding) was determined by replacing compound with 0.25% of DMSO (dimethyl sulfoxide) in the reaction mixture. Specific binding data were obtained by subtracting the radioactivity of non-specific binding from each binding data.

The IC₅₀ value is defined as the concentration of the tested compound that is required for 50% inhibition of total specific binding. In order to calculate the IC₅₀ value, the percent inhibition data were determined for 7 concentrations for each compound. The percent inhibition for a compound at each concentration was calculated according to the following formula, [1−(specific binding in the presence of compound)/(total specific binding)]×100. The IC₅₀ value was then obtained by fitting the percent inhibition data to a sigmoidal dose-response (4 parameter logistic) model in the XLfit® software Excel add-in program [from ID Business Solutions Ltd., model 205, where F(x)=(A+(B−A)/(1+((C/x)̂D)))].

The acid compounds of the foregoing examples were tested using the above Human CRTH2 Receptor Binding Assay (examples 1-1 to 1-9, 2-1, 3-1 to 3-3, 4-1 to 4-3, 5-1, 6-1, 7-1, 8-1, 9-1 to 9-3, and 10-1 to 10-12). The results of the assay showed that all of these compounds have binding activity exhibiting IC₅₀ values ranging from 0.0029 μM to 3.25 μM. For instance, the following table shows the specific IC₅₀ values for these compounds:

| | Human CRTH2 Binding |
|---|---|
| Example No. | IC₅₀ (μM) |
| Example 1-1 | 0.3810 |
| Example 1-2 | 0.1771 |
| Example 1-3 | 0.0157 |
| Example 1-4 | 0.1101 |
| Example 1-5 | 0.0742 |
| Example 1-6 | 0.0183 |
| Example 1-7 | 0.4560 |
| Example 1-8 | 3.2500 |
| Example 1-9 | 2.5800 |
| Example 2-1 | 0.0068 |
| Example 3-1 | 0.0029 |
| Example 3-2 | 0.0036 |
| Example 3-3 | 0.0034 |
| Example 4-1 | 0.1266 |
| Example 4-2 | 0.7730 |
| Example 4-3 | 0.4100 |
| Example 5-1 | 0.0165 |
| Example 6-1 | 0.0782 |
| Example 7-1 | 0.0766 |
| Example 8-1 | 0.0912 |
| Example 9-1 | 0.1469 |
| Example 9-2 | 0.3990 |
| Example 9-3 | 0.4230 |
| Example 10-1 | 0.3400 |
| Example 10-2 | 0.0060 |
| Example 10-3 | 0.0063 |
| Example 10-4 | 0.3100 |
| Example 10-5 | 0.0130 |
| Example 10-6 | 0.0135 |
| Example 10-7 | 0.0184 |
| Example 10-8 | 0.0310 |
| Example 10-9 | 0.0090 |
| Example 10-10 | 0.0180 |
| Example 10-11 | 0.0090 |
| Example 10-12 | 0.0200 |

### Calcium Flux Assay Using Fluorometric Imaging Plate Reader

### Cell Culture Conditions:

CHO-K1 cells previously transfected with G-alpha 16 were subsequently transfected with the human CRTH2 receptor and the neomycin resistance gene. Following selection in 800 μg/mL G418 (geneticin), individual clones were assayed for their receptor expression based on staining with an anti human CRTH2 IgG, followed by assaying for their response to 13,14-dihydro-15-keto Prostaglandin D₂ (DK-PDG₂) (ligand) in the Ca²⁺ Flux assay. Positive clones were then cloned by limiting dilution cloning. The transfected cells were cultured in Ham's F-12 medium supplemented with 10% fetal bovine serum, 2 mM glutamine, 100 U/mL penicillin/100 μg/mL streptomycin, 200 μg/mL hygromycin B, and 800 μg/mL G418 (geneticin). Cells were harvested with trypsin-EDTA (trypsin-ethylenediaminetetraacetic acid) and counted using ViaCount® reagent (from Guava Technologies, Inc. which contains two DNA-binding dyes that enable the reagent user to distinguish between viable and non-viable cells). The cell suspension volume was adjusted to 2.5×10⁵ cells /mL with complete growth media. Aliquots of 50 μL were dispensed into BD Falcon™ 384 well black/clear microplates (from BD Biosciences, a division of Becton, Dickinson and Company) and the microplates were placed in a 37° C. CO₂ incubator overnight. The following day, the microplates were used in the assay.

### Dye Loading and Assay:

Loading Buffer containing dye (from the FLIPR® Calcium 3 Assay Kit from Molecular Devices, a division of MDS Analytical Technologies and MDS Inc.) was prepared by dissolving the contents of one bottle into 200 mL Hank's Balanced Salt Solution containing 20 mM HEPES (4-(2-hydroxyethyl)-1-piperazineethanesulfonic acid) and 2.5 mM probenecid. Growth media was removed from the cell plates and 25 μL of Hank's Balanced Salt Solution (HBSS) containing 20 mM HEPES, 0.05% BSA and 2.5 mM probenecid was added to each well followed by 25 μL of diluted dye using a Multidrop dispenser. The plates were then incubated for 1 hour at 37° C.

During the incubation, test compound plates were prepared by adding 90 μL of HBSS/20 mM HEPES/0.005% BSA buffer to the 2 μL of serial diluted compounds. To prepare serial diluted compounds, 20 mM stocks of compounds were dissolved in 100% DMSO. The compound dilution plate was set up as follows: well #1 received 5 μL of compound plus 10 μL of DMSO. Wells 2-10 received 10 μL of DMSO. 5 μL was mixed and transferred from well #1 into well #2. 1:3 serial dilutions were continued out 10 steps. 2 μL of diluted compound was transferred into duplicate wells of a 384 well “assay plate” and then 90 μL of buffer was added.

After incubation, both the cell and “assay plate” plates were brought to the fluorometric imaging plate reader (FLIPR®) and 20 μL of the diluted compounds were transferred to the cell plates by the FLIPR®. Plates were then incubated for 1 hour at room temperature. After the 1 hour incubation, plates were returned to the FLIPR® and 20 μL of 4.5× concentrated ligand was added to the cell plates. During the assay, fluorescence readings were taken simultaneously from all 384 wells of the cell plate every 1.5 seconds. Five readings were taken to establish a stable baseline, then 20 μL of sample was rapidly (30 μL/sec) and simultaneously added to each well of the cell plate. The fluorescence was continuously monitored before, during and after sample addition for a total elapsed time of 100 seconds. Responses (increase in peak fluorescence) in each well following agonist addition were determined. The initial fluorescence reading from each well, prior to ligand stimulation, was used as a zero baseline value for the data from that well. The responses were expressed as % inhibition of the buffer control. The IC₅₀ value, defined as the concentration of a compound that was required for 50% inhibition of the buffer control, was calculated by fitting the percent inhibition data for 10 concentrations to a sigmoidal dose-response (4 parameter logistic) model using Genedata Screener® Condoseo software program [from Genedata AG, model 205, where F(x)=(A+(B−A)/(1+((C/x)̂D)))].

The compounds tested in the above FLIPR® assay were examples 1-1 to 1-6, 2-1, 3-1 to 3-3, 4-1 to 4-3, 5-1, 6-1, 7-1, 8-1, 9-1, 9-3, 10-1 to 10-3, and 10-5 to 10-12). The results of the FLIPR® assay showed that, with the exception of example 10-1 (which exhibited an IC₅₀ value of approximately 3), all of the representative compounds tested in this assay exhibited IC₅₀ values ranging from 0.0001 μM to 2.01 μM. For instance, example 1-1 exhibited an IC₅₀ value of 1.77 μM, example 4-2 exhibited an IC₅₀ value of 2.01 μM, example 9-3 exhibited an IC₅₀ value of 0.462 μM, example 10-5 exhibited an IC₅₀ value of 0.094 μM, and example 10-12 exhibited an IC₅₀ value of 0.313 μM.

### DK-PGD₂-Induced IL-13 Production Assay in Th2 Cells

Inhibition of 13,14-dihydro-15-keto Prostaglandin D₂ (DK-PGD₂)-induced IL-13 production in T helper type 2 (Th2) cells was applied to evaluate compound cellular potency.

Cultures of Th2 cells were established from blood of healthy human volunteers according to the following procedure. Peripheral blood mononuclear cells (PBMC) were first isolated from 50 mL of fresh blood by Ficoll-Hypaque density gradient centrifugation, followed by CD4⁺ cell purification using a CD4⁺ T Cell Isolation Kit II (from Miltenyi Biotec Inc.). The CD4⁺ T cells were then differentiated to Th2 cells by culturing the cells in X-VIVO 15® medium (from Cambrex BioScience Walkersville Inc.) containing 10% human AB serum (serum of blood type AB from Invitrogen Corporation), 50 U/mL of recombinant human interleukin-2 (rhIL-2) (from PeproTech Inc.) and 100 ng/mL of recombinant human interleukin-4 (rhIL-4) (from PeproTech Inc.) for 7 days. The Th2 cells were isolated using a CD294 (CRTH2) MicroBead Kit (from Miltenyi Biotec Inc.) and amplified in X-VIVO 15® medium containing 10% human AB serum and 50 U/mL of rhIL-2 for 2 to 5 weeks. In general, 70% to 80% of the Th2 cells used in the assay are CRTH2-positive when analyzed by fluorescence-activated cell sorting using the BM16 antibody (as previously described) conjugated to phycoerythrin (PE).

To determine cellular inhibitory potency, compounds at various concentrations were incubated with 2.5×10⁴ Th2 cells and 500 nM DK-PGD₂ for 4 hrs at 37° C. in 200 μL of X-VIVO 15® medium containing 10% human AB serum. IL-13 production to the medium was detected by ELISA (enzyme-linked immunosorbent assay) using an “Instant ELISA™” kit (from Bender MedSystems Inc.) according to the procedure suggested by the vendor. The spontaneous production of IL-13 by Th2 cells was determined in the absence of DK-PGD2 stimulation and the value was subtracted from that in the presence of each compound for percent inhibition and IC₅₀ calculations.

The percent inhibition of interleukin 13 (IL-13) production for a compound at various concentrations was calculated according to the following formula, [1-(IL-13 production in the presence of compound)/(IL-13 production in the presence of 0.15% DMSO)]×100. The IC₅₀ value, defined as the concentration of a compound that is required for 50% inhibition of IL-13 production, was calculated by fitting the percent inhibition data for 7 concentrations to a sigmoidal dose-response (4 parameter logistic) model in the XLfit® software Excel add-in program [ID Business Solutions Ltd., model 205, where F(x)=(A+(B−A)/(1+((C/x)̂D)))].

The compounds tested using the foregoing DK-PGD₂-induced IL-13 production assay were examples 1-1 to 1-9, 2-1, 3-1 to 3-3, 4-1 to 4-3, 5-1, 6-1, 7-1, 8-1, 9-1 to 9-3, 10-2, 10-3, and 10-6. The results of the DK-PGD₂-induced IL-13 production assay showed that, with the exception of examples 1-8 and 1-9 (which exhibited IC₅₀ values greater than 10), the compounds tested in this assay exhibited activity in inhibiting IL-13 production, with IC₅₀ values ranging from 0.0032 μM to 6.428 μM. For instance, example 1-1 exhibited an IC₅₀ value of 4.645 μM, example 1-7 exhibited an IC₅₀ value of 6.428 μM, example 4-2 exhibited an IC₅₀ value of 3.014 μM, example 9-2 exhibited an IC₅₀ value of 4.845 μM, and example 9-3 exhibited an IC₅₀ value of 5.09 μM.

Thus, the compounds of the present invention are useful since the compounds tested show some activity in at least one of the above three assays (i.e., binding at the CRTH2 receptor), and therefore may be useful as antagonists in treating diseases and disorders associated with this receptor such as asthma.

### Human Thromboxane A2 Receptor Binding Assay

The thromboxane A2 receptor (TP) plays a key role in hemostasis as its abnormality leads to bleeding disorders. To avoid the potential liability of bleeding disorders, the binding activity of certain compounds of the present invention against TP was monitored by a receptor binding assay using human platelets as the source of the receptor and [³H]SQ29548 (generically named (5Z)-[5,6-³H]-7-[(1S,2R,3R,4R)-3-[[2-[(phenylamino)carbonyl]hydrazinyl]methyl]-7-oxabicyclo[2.2.1]hept-2-yl]-5-heptenoic acid, from PerkinElmer Inc.) as the competing radioactive ligand.

The TP binding reactions (0.2 mL) were performed in 96-well plates at room temperature in PBS containing 5×10⁷ platelets, 10 mM MgCl₂, 0.06% BSA, 10 nM [³H]SQ29548, and the test compound at various concentrations. After 1 hour of binding reactions, the platelets were harvested on GF/B filter plates (as previously described from PerkinElmer Inc.) and washed 5 times with PBS using a Filtermate™ Harvester (as previously described from PerkinElmer Inc.). The radioactivities bound to the platelets were determined using a microplate scintillation counter (TopCount® NXT, from PerkinElmer Inc.) after adding 50 μL of Microscint™ 20 scintillation fluid (from PerkinElmer Inc.) to each well of the filter plates. The radioactivity from non-specific binding was determined by replacing the compound with 10 μM of ramatroban (BAY-u3405, from Cayman Chemical Company) in the reaction mixtures. The radioactivity bound to the platelets in the absence of compound (total binding) was determined by replacing the compound with 0.25% of DMSO in the reaction mixture. Specific binding data were obtained by subtracting the radioactivity of non-specific binding from each binding data.

The IC₅₀ value is defined as the concentration of the tested compound that is required for 50% inhibition of total specific binding. In order to calculate the IC₅₀ value, the percent inhibition data were determined for 7 concentrations for each compound. The percent inhibition for a compound at each concentration was calculated according to the following formula, [1−(specific binding in the presence of compound)/(total specific binding)]×100. The IC₅₀ value was then obtained by fitting the percent inhibition data to a sigmoidal dose-response (4 parameter logistic) model in the XLfit® software Excel add-in program [from ID Business Solutions Ltd., model 205, where F(x)=(A+(B−A)/(1+((C/x)̂D)))].

The results of the thromboxane A2 receptor binding assay are summarized in the following table:

| | Thromboxane A2 |
|---|---|
| | Receptor Binding |
| Example No. | IC₅₀ (μM) |
| Example 1-1 | >10 |
| Example 1-2 | >10 |
| Example 1-3 | >10 |
| Example 1-4 | >10 |
| Example 1-5 | >10 |
| Example 1-6 | >10 |
| Example 1-7 | >10 |
| Example 1-8 | 2.145 |
| Example 1-9 | >10 |
| Example 2-1 | >10 |
| Example 3-1 | >10 |
| Example 3-2 | >10 |
| Example 3-3 | >10 |
| Example 4-1 | >10 |
| Example 4-2 | >10 |
| Example 4-3 | >10 |
| Example 5-1 | >10 |
| Example 6-1 | >10 |
| Example 7-1 | >10 |
| Example 8-1 | >10 |
| Example 9-1 | >10 |
| Example 9-2 | >10 |
| Example 9-3 | 8.524 |
| Example 10-1 | 0.1260 |
| Example 10-2 | >10 |
| Example 10-3 | >10 |
| Example 10-4 | 8.179 |
| Example 10-5 | 2.421 |
| Example 10-6 | >10 |
| Example 10-7 | >10 |
| Example 10-8 | >10 |
| Example 10-9 | >10 |
| Example 10-10 | >10 |
| Example 10-11 | >10 |
| Example 10-12 | 0.3420 |

The results of the thromboxane A2 receptor binding assay indicate that (with perhaps the exception of Example 1-8, 10-1, 10-5, and 10-12) the compounds tested generally do not bind to the thromboxane A2 receptor to the extent that such compounds would be considered to be thromboxane A2 antagonists having a significant anti-aggregating effect on blood platelets.

The present invention is also directed to a use for the compounds of formula I as therapeutically active substances and, in particular, to a method for the treatment or prevention of diseases or disorders which are associated with the CRTH2 receptor.

In one embodiment, the present invention relates to a method for the treatment and/or prevention of diseases and disorders which are associated with the modulation of CRTH2 receptors, which method comprises administering a therapeutically effective amount of a compound of formula I to a human being or animal. A method for the treatment and/or prevention of an inflammatory or allergic disease or disorder is preferred. Such diseases or disorders include (but are not limited to) asthma, chronic obstructive pulmonary disease (COPD), allergic rhinitis, allergic inflammation, and atopic dermatitis.

The present invention is also directed to the administration of a therapeutically effective amount of a compound of formula I in combination or association with other drugs or active agents for the treatment of inflammatory or allergic diseases and disorders. In one embodiment, the present invention relates to a method for the treatment and/or prevention of such diseases or disorders comprising administering to a human or animal simultaneously, sequentially, or separately, a therapeutically effective amount of a compound of formula I and another drug or active agent (such as another anti-inflammatory or anti-allergic drug or agent). These other drugs or active agents may have the same, similar, or a completely different mode of action. Suitable other drugs or active agents may include, but are not limited to: Beta2-adrenergic agonists such as albuterol or salmeterol; corticosteroids such as dexamethasone or fluticasone; antihistamines such as loratidine; leukotriene antagonists such as montelukast or zafirlukast; anti-IgE antibody therapies such as omalizumab; anti-infectives such as fusidic acid (particularly for the treatment of atopic dermatitis); anti-fungals such as clotrimazole (particularly for the treatment of atopic dermatitis); immunosuppressants such as tacrolimus and pimecrolimus; other antagonists of PGD2 acting at other receptors such as DP antagonists; inhibitors of phoshodiesterase type 4 such as cilomilast; drugs that modulate cytokine production such as inhibitors of TNF-alpha converting enzyme (TACE); drugs that modulate the activity of Th2 cytokines IL-4 and IL-5 such as blocking monoclonal antibodies and soluble receptors; PPAR-gamma agonists such as rosiglitazone; and 5-lipoxygenase inhibitors such as zileuton.

Unless stated to the contrary, all compounds in the examples were prepared and characterized as described. All patents and publications cited herein are hereby incorporated by reference in their entirety.

1. A compound of formula I: 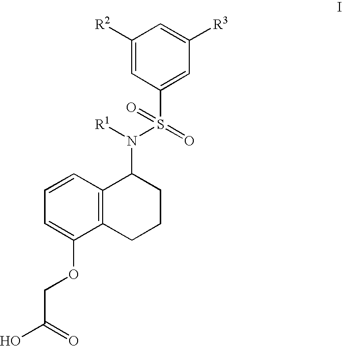
or a pharmaceutically acceptable salt or ester thereof, wherein R¹ is hydrogen or methyl, and R² and R³ are independently selected from the group consisting of:
(1) halogen;
(2) —NH₂;
(3) —NO₂;
(4) lower alkyl optionally substituted by fluoro,
(5) lower cycloalkyl optionally substituted by lower alkyl;
(6) lower alkenyl;
(7) lower alkanoyl;
(8) lower alkoxy;
(9) lower cycloalkoxy;
(10) lower heterocycloalkyl;
(11) lower alkylsulfanyl, lower cycloalkylsulfanyl, or lower heterocycloalkylsulfanyl;
(12) lower alkylsulfinyl, lower cycloalkylsulfinyl, or lower heterocycloalkylsulfinyl;
(13) lower alkylsulfonyl, lower cycloalkylsulfonyl, or lower heterocycloalkylsulfonyl;
(14) lower alkylsulfonylamino;
(15) lower alkylamino;
(16) lower dialkylamino; and
(17) lower trialkylsilyl.

2. A compound of claim 1 wherein R¹ is hydrogen.

3. A compound of claim 1 wherein R¹ is methyl.

4. A compound of claim 1 which is an (R)-enantiomer as depicted in formula IA: 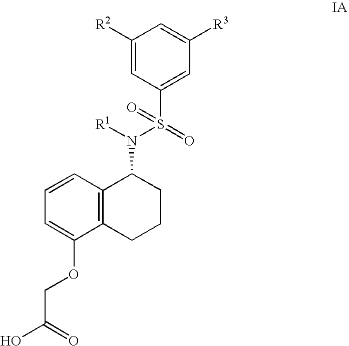
wherein R¹, R² and R³ are as defined in claim 1.

5. A compound of claim 4 wherein R¹ is hydrogen.

6. A compound of claim 4 wherein R¹ is methyl.

7. A compound of claim 4 wherein R² and R³ are independently selected from the group consisting of:
(1) halogen;
(2) lower alkyl;
(3) lower alkyl substituted by fluoro;
(4) cycloalkyl;
(5) lower cycloalkyl substituted by lower alkyl;
(6) lower heterocycloalkyl;
(7) lower alkanoyl;
(8) lower alkoxy;
(9) lower cycloalkoxy;
(10) lower alkylsulfinyl;
(11) lower alkylsulfonyl;
(12) lower cycloalkylsulfonyl;
(13) lower alkylamino; and
(14) lower dialkylamino.

8. A compound of claim 7 wherein at least one of R² or R³ is fluoro, chloro, or bromo.

9. A compound of claim 7 wherein at least one of R² or R³ is methyl, ethyl, propyl, isopropyl, butyl, isobutyl, sec-butyl, or tert-butyl.

10. A compound of claim 7 wherein at least one of R² or R³ is trifluoromethyl, difluoromethyl, 1,1-difluoroethyl, or fluoromethyl.

11. A compound of claim 7 wherein at least one of R² or R³ is cyclopropyl, cyclobutyl, cyclopentyl, or cyclohexyl.

12. A compound of claim 7 wherein at least one of R² or R³ is methoxy, ethoxy, n-propoxy, isopropoxy, n-butoxy, isobutoxy, sec-butoxy, or tert-butoxy.

13. A compound of claim 7 wherein at least one of R² or R³ is cyclobutoxy or cyclopentoxy.

14. A compound of claim 7 wherein at least one of R² or R³ is methylsulfinyl, ethylsulfinyl, isopropylsulfinyl, methylsulfonyl, ethylsulfonyl, isopropylsulfonyl, tert-butylsulfonyl, cyclopropylsulfonyl, cyclobutylsulfonyl, or cyclopentylsulfonyl.

15. A compound of claim 7 wherein at least one of R² or R³ is trifluoromethyl.

16. A compound of claim 1 selected from the group consisting of:
{(R)-5-[(3-fluoro-5-trifluoromethyl-benzenesulfonyl)-methyl-amino]-5,6,7,8-tetrahydro-naphthalen-1-yloxy}-acetic acid;
{(R)-5-[(3,5-di-tert-butyl-benzenesulfonyl)-methyl-amino]-5,6,7,8-tetrahydro-naphthalen-1-yloxy}-acetic acid;
{(R)-5-[(3,5-bis-methanesulfonyl-benzenesulfonyl)-methyl-amino]-5,6,7,8-tetrahydro-naphthalen-1-yloxy}-acetic acid;
{(R)-5-[(3-methoxy-5-trifluoromethyl-benzenesulfonyl)-methyl-amino]-5,6,7,8-tetrahydro-naphthalen-1-yloxy}-acetic acid;
{(R)-5-[(3-bromo-5-trifluoromethyl-benzenesulfonyl)-methyl-amino]-5,6,7,8-tetrahydro-naphthalen-1-yloxy}-acetic acid;
{(R)-5-[(3,5-bis-trifluoromethyl-benzenesulfonyl)-methyl-amino]-5,6,7,8-tetrahydro-naphthalen-1-yloxy}-acetic acid;
{(R)-5-[(3,5-dichloro-benzenesulfonyl)-methyl-amino]-5,6,7,8-tetrahydro-naphthalen-1-yloxy}-acetic acid;
{(R)-5-[(3,5-difluoro-benzenesulfonyl)-methyl-amino]-5,6,7,8-tetrahydro-naphthalen-1-yloxy}-acetic acid;
{(R)-5-[(3,5-dimethyl-benzenesulfonyl)-methyl-amino]-5,6,7,8-tetrahydro-naphthalen-1-yloxy}-acetic acid;
((R)-5-{methyl-[3-(propane-2-sulfinyl)-5-trifluoromethyl-benzenesulfonyl]-amino}-5,6,7,8-tetrahydro-naphthalen-1-yloxy)-acetic acid;
{(R)-5-[(3-cyclopentanesulfonyl-5-trifluoromethyl-benzenesulfonyl)-methyl-amino]-5,6,7,8-tetrahydro-naphthalen-1-yloxy}-acetic acid;
((R)-5-{methyl-[3-(propane-2-sulfonyl)-5-trifluoromethyl-benzenesulfonyl]-amino}-5,6,7,8-tetrahydro-naphthalen-1-yloxy)-acetic acid;
((R)-5-{methyl-[3-(2-methyl -propane-2-sulfonyl)-5-trifluoromethyl-benzenesulfonyl]-amino}-5,6,7,8-tetrahydro-naphthalen-1-yloxy)-acetic acid;
{(R)-5-[methyl-(3-pyrrolidin-1-yl-5-trifluoromethyl-benzenesulfonyl)-amino]-5,6,7,8-tetrahydro-naphthalen-1-yloxy}-acetic acid;
{(R)-5-[(3-diethylamino-5-trifluoromethyl-benzenesulfonyl)-methyl-amino]-5,6,7,8-tetrahydro-naphthalen-1-yloxy}-acetic acid;
((R)-5-{[3-(isopropyl-methyl-amino)-5-trifluoromethyl-benzenesulfonyl]-methyl-amino}-5,6,7,8-tetrahydro-naphthalen-1-yloxy)-acetic acid;
((R)-5-{[3-(1,1-difluoro-ethyl)-5-trifluoromethyl-benzenesulfonyl]-methyl-amino}-5,6,7,8-tetrahydro-naphthalen-1-yloxy)-acetic acid;
{(R)-5-[(3-acetyl-5-trifluoromethyl-benzenesulfonyl)-methyl-amino]-5,6,7,8-tetrahydro-naphthalen-1-yloxy}-acetic acid;
((R)-5-{methyl-[3-(1-methyl-cyclopropyl)-5-trifluoromethyl-benzenesulfonyl]-amino}-5,6,7,8-tetrahydro-naphthalen-1-yloxy)-acetic acid;
{(R)-5-[(3-isopropyl-5-trifluoromethyl-benzenesulfonyl)-methyl-amino]-5,6,7,8-tetrahydro-naphthalen-1-yloxy}-acetic acid;
{(R)-5-[(3-isopropoxy-5-trifluoromethyl-benzenesulfonyl)-methyl-amino]-5,6,7,8-tetrahydro-naphthalen-1-yloxy}-acetic acid;
{(R)-5-[(3-ethoxy-5-trifluoromethyl-benzenesulfonyl)-methyl-amino]-5,6,7,8-tetrahydro-naphthalen-1-yloxy}-acetic acid;
{(R)-5-[(3-cyclopentyloxy-5-trifluoromethyl-benzenesulfonyl)-methyl-amino]-5,6,7,8-tetrahydro-naphthalen-1-yloxy}-acetic acid;
{(R)-5-[(3,5-di-tert-butyl-benzenesulfonyl)-methyl-amino]-5,6,7,8-tetrahydro-naphthalen-1-yloxy}-acetic acid;
{(R)-5-[(3,5-bis-methanesulfonyl-benzenesulfonyl)-methyl-amino]-5,6,7,8-tetrahydro-naphthalen-1-yloxy}-acetic acid;
{(R)-5-[(3-methoxy-5-trifluoromethyl-benzenesulfonyl)-methyl-amino]-5,6,7,8-tetrahydro-naphthalen-1-yloxy}-acetic acid;
{(R)-5-[(3-bromo-5-trifluoromethyl-benzenesulfonyl)-methyl-amino]-5,6,7,8-tetrahydro-naphthalen-1-yloxy}-acetic acid;
{(R)-5-[(3,5-bis-trifluoromethyl-benzenesulfonyl)-methyl-amino]-5,6,7,8-tetrahydro-naphthalen-1-yloxy}-acetic acid;
((R)-5-{methyl-[3-(propane-2-sulfinyl)-5-trifluoromethyl-benzenesulfonyl]-amino}-5,6,7,8-tetrahydro-naphthalen-1-yloxy)-acetic acid;
{(R)-5-[(3-cyclopentanesulfonyl-5-trifluoromethyl-benzenesulfonyl)-methyl-amino]-5,6,7,8-tetrahydro-naphthalen-1-yloxy}-acetic acid;
((R)-5-{methyl-[3-(propane-2-sulfonyl)-5-trifluoromethyl-benzenesulfonyl]-amino}-5,6,7,8-tetrahydro-naphthalen-1-yloxy)-acetic acid;
((R)-5-{methyl-[3-(2-methyl-propane-2-sulfonyl)-5-trifluoromethyl-benzenesulfonyl]-amino}-5,6,7,8-tetrahydro-naphthalen-1-yloxy)-acetic acid;
{(R)-5-[methyl-(3-pyrrolidin-1-yl-5-trifluoromethyl-benzenesulfonyl)-amino]-5,6,7,8-tetrahydro-naphthalen-1-yloxy}-acetic acid;
((R)-5-{[3-(isopropyl-methyl-amino)-5-trifluoromethyl-benzenesulfonyl]-methyl-amino}-5,6,7,8-tetrahydro-naphthalen-1-yloxy)-acetic acid;
((R)-5-{[3-(1,1-difluoro-ethyl)-5-trifluoromethyl-benzenesulfonyl]-methyl-amino}-5,6,7,8-tetrahydro-naphthalen-1-yloxy)-acetic acid;
{(R)-5-[(3-acetyl-5-trifluoromethyl-benzenesulfonyl)-methyl-amino]-5,6,7,8-tetrahydro-naphthalen-1-yloxy}-acetic acid;
((R)-5-{methyl-[3-(1-methyl-cyclopropyl)-5-trifluoromethyl-benzenesulfonyl]-amino}-5,6,7,8-tetrahydro-naphthalen-1-yloxy)-acetic acid;
{(R)-5-[(3-isopropyl-5-trifluoromethyl-benzenesulfonyl)-methyl-amino]-5,6,7,8-tetrahydro-naphthalen-1-yloxy}-acetic acid;
{(R)-5-[(3-isopropoxy-5-trifluoromethyl-benzenesulfonyl)-methyl-amino]-5,6,7,8-tetrahydro-naphthalen-1-yloxy}-acetic acid; and
{(R)-5-[(3-ethoxy-5-trifluoromethyl-benzenesulfonyl)-methyl-amino]-5,6,7,8-tetrahydro-naphthalen-1-yloxy}-acetic acid.

17. A pharmaceutically acceptable salt or ester of a compound of claim 16.

18. A compound of claim 1 selected from the group consisting of:
[(R)-5-(3,5-dichloro-benzenesulfonylamino)-5,6,7,8-tetrahydro-naphthalen-1-yloxy]-acetic acid;
[(R)-5-(3,5-bis-trifluoromethyl-benzenesulfonylamino)-5,6,7,8-tetrahydro-naphthalen-1-yloxy]-acetic acid;
[(R)-5-(3,5-dimethyl-benzenesulfonylamino)-5,6,7,8-tetrahydro-naphthalen-1-yloxy]-acetic acid;
[(R)-5-(3,5-difluoro-benzenesulfonylamino)-5,6,7,8-tetrahydro-naphthalen-1-yloxy]-acetic acid;
[(R)-5-(3-isopropyl-5-trifluoromethyl-benzenesulfonylamino)-5,6,7,8-tetrahydro-naphthalen-1-yloxy]-acetic acid;
{(R)-5-[3-(1,1-difluoro-ethyl)-5-trifluoromethyl-benzenesulfonylamino]-5,6,7,8-tetrahydro-naphthalen-1-yloxy}-acetic acid;
{(R)-5-[3-(1-methyl-cyclopropyl)-5-trifluoromethyl-benzenesulfonylamino]-5,6,7,8-tetrahydro-naphthalen-1-yloxy}-acetic acid;
[(R)-5-(3,5-di-tert-butyl-benzenesulfonylamino)-5,6,7,8-tetrahydro-naphthalen-1-yloxy]-acetic acid;
[(R)-5-(3,5-bis-methanesulfonyl-benzenesulfonylamino)-5,6,7,8-tetrahydro-naphthalen-1-yloxy]-acetic acid;
[(R)-5-(3-methoxy-5-trifluoromethyl-benzenesulfonylamino)-5,6,7,8-tetrahydro-naphthalen-1-yloxy]-acetic acid;
[(R)-5-(3-bromo-5-trifluoromethyl-benzenesulfonylamino)-5,6,7,8-tetrahydro-naphthalen-1-yloxy]-acetic acid;
[(R)-5-(3-fluoro-5-trifluoromethyl-benzenesulfonylamino)-5,6,7,8-tetrahydro-naphthalen-1-yloxy]-acetic acid;
[(R)-5-(3,5-bis-trifluoromethyl-benzenesulfonylamino)-5,6,7,8-tetrahydro-naphthalen-1-yloxy]-acetic acid;
[(R)-5-(3-isopropyl-5-trifluoromethyl-benzenesulfonylamino)-5,6,7,8-tetrahydro-naphthalen-1-yloxy]-acetic acid;
{(R)-5-[3-(1,1-difluoro-ethyl)-5-trifluoromethyl-benzenesulfonylamino]-5,6,7,8-tetrahydro-naphthalen-1-yloxy}-acetic acid;
{(R)-5-[3-(1-methyl-cyclopropyl)-5-trifluoromethyl-benzenesulfonylamino]-5,6,7,8-tetrahydro-naphthalen-1-yloxy}-acetic acid;
[(R)-5-(3,5-di-tert-butyl-benzenesulfonylamino)-5,6,7,8-tetrahydro-naphthalen-1-yloxy]-acetic acid;
[(R)-5-(3,5-bis-methanesulfonyl-benzenesulfonylamino)-5,6,7,8-tetrahydro-naphthalen-1-yloxy]-acetic acid;
[(R)-5-(3-methoxy-5-trifluoromethyl-benzenesulfonylamino)-5,6,7,8-tetrahydro-naphthalen-1-yloxy]-acetic acid; and
[(R)-5-(3-bromo-5-trifluoromethyl-benzenesulfonylamino)-5,6,7,8-tetrahydro-naphthalen-1-yloxy]-acetic acid.

19. A pharmaceutically acceptable salt or ester of a compound of claim 18.

20. A compound of claim 1 which is {(R)-5-[(3,5-bis-trifluoromethyl-benzenesulfonyl)-methyl-amino]-5,6,7,8-tetrahydro-naphthalen-1-yloxy}-acetic acid.

21. A pharmaceutical composition comprising a therapeutically effective amount of a compound of claim 1 and a pharmaceutically acceptable carrier.
